(19) United States
(12) Patent Application Publication
(10) Pub. No.: US 2020/0016201 A1
(43) Pub. Date: Jan. 16, 2020

(54) CHIMERIC ANTIGEN RECEPTORS AND COMPOSITIONS AND METHODS OF USE THEREOF

(71) Applicant: University of Southern California, Los Angeles, CA (US)

(72) Inventor: Alan L. Epstein, Los Angeles, CA (US)

(21) Appl. No.: 16/335,570

(22) PCT Filed: Sep. 22, 2017
(86) PCT No.: PCT/US2017/052974

§ 371 Date: Mar. 21, 2019

Related U.S. Application Data
(60) Provisional application No. 62/399,244, filed on Sep. 23, 2016.

(51) Int. Cl. A61K 35/17; C07K 14/725; C07K 14/705; C07K 14/72; C07K 16/28; C07K 16/30

(52) U.S. Cl. CPC A61K 35/17; C07K 2319/33; C07K 14/70517; C07K 14/70521; C07K 14/70532; C07K 14/70578; C07K 14/723; C07K 16/2833; C07K 16/2869; C07K 16/3092; C07K 2317/622; C07K 2317/73; C07K 2319/03; C07K 2319/30; C07K 14/7051

(57) ABSTRACT

Disclosed herein are novel chimeric antigen receptors (CARs) targeting human LHR, B7-H4, HLA-G, or HLA-DR, and therapeutic methods of their use. LHR, B7-H4, HLA-G, or HLA-DR are expressed in the context of many human cancers including thyroid, prostate, colon, breast, ovarian, and renal cancers, as well as B-cell leukymias and lymphomas.

### CROSS REFERENCE TO RELATED APPLICATION

The present application claims priority under 35 U.S.C. § 119(e) to U.S. Provisional Application 62/399,244, filed on Sep. 23, 2016, the contents of which are hereby incorporated by reference in its entirety.

### TECHNICAL FIELD

This disclosure relates to novel luteinizing hormone receptor (LHR), B7-H4, HLA-G, or HLA-DR chimeric antigen receptor (CAR), cells or compositions comprising the same, and methods for using the same for therapy including solid tumors. Also provided herein are isolated peptides and fusion proteins containing immunogenic determinants for the luteinizing hormone receptor, B7-H4, HLA-G, or HLA-DR chimeric antigen receptor.

### BACKGROUND

The following discussion of the background of the disclosure is merely provided to aid the reader in the understanding the invention and is not admitted to describe or constitute prior art to the present invention.

Ovarian carcinoma is the most common cause of cancer death from gynecologic tumors (Siegel, R. et al. (2012) CA Cancer J. Clin. 62:10-29). Approximately 25,000 new cases and 14,000 deaths are expected to occur in the United States every year (Siegel, R. et al. (2012) CA Cancer J. Clin. 62:10-29). Overall survival of ovarian carcinoma appears to have improved in the last 30 years as median survival during the 1960s was approximately 12 months compared to the current 38 months. However, the 5-year survival for stage III ovarian cancer has not changed significantly and remains at 25%. The improvement in median survival can be explained in part due to the improvement in front line chemotherapy. The standard initial chemotherapy for patients with ovarian cancer involves a platinum-paclitaxel based regimen (Marcus, C. S. et al. (2014) J. Cancer 5:25-30). Approximately 70% of patients will achieve a clinical response to this therapy. Despite this, most women will relapse and eventually succumb to their disease. Therefore, in an attempt to decrease distant metastasis, prolong time to recurrence and improve overall survival, it is essential to identify novel therapy targets and develop new agents.

In 2014, an estimated 232,670 new agents.cases of invasive breast cancer will be diagnosed in US women and an estimated 40,000 US women will die from metastatic disease. The risk of contracting breast cancer increases with age so that 77% of cases are over the age of 50 at the time of diagnosis. In general, the mortality rate for patients with breast cancer has decreased since 1989 due to earlier detection, improved treatments, and possibly a decreased incidence because of the declining use of postmenopausal hormone therapy. When detected early, the 5-year survival for localized breast cancer is 99%. By contrast, the 5-year survival for regional disease is 84% and importantly, for metastatic disease, it drops precipitously to 24%.

This year, an estimated 63,920 adults (39,140 men and 24,780 women) in the United States will be diagnosed with renal cancer. It is estimated that 13,860 deaths (8,900 men and 4,960 women) from this disease will occur this year. Renal cancer is the sixth most common cancer and the tenth most common cause of cancer death for men, and it is the eighth most common cause of cancer for women. The five-year survival rate for renal cancer patients is 72%. Approximately 63% of cases do not have metastatic disease at the time of diagnosis. For this group, the five-year survival rate improves to 92%. By contrast, the five-year survival for renal cancer in the pelvis (metastatic disease) is 51%.

Therefore, a need exists for a safe and effective treatment of ovarian and other solid tumor cancers, e.g., prostate cancer. This disclosure satisfies this need and provides related advantages as well.

### SUMMARY OF THE DISCLOSURE

Due to the unprecedented results being recently obtained in B-cell lymphomas and leukemias using autologous treatment with genetically engineered chimeric antigen receptor (CAR) T-cells, a number of laboratories have begun to apply this approach to solid tumors including ovarian cancer. CAR modified T-cells combine the HLA-independent targeting specificity of a monoclonal antibody with the cytolytic activity, proliferation, and homing properties of activated T-cells, but do not respond to checkpoint suppression. Because of their ability to kill antigen expressing targets directly, CAR T-cells are highly toxic to any antigen positive cells or tissues making it a requirement to construct CARs with highly tumor specific antibodies. To date, CAR modified T-cells to ovarian carcinomas have been constructed against the α-folate receptor, mesothelin, and MUC-CD, but all of these have some off-target expression of antigen.

For instance, in one aspect, disclosed herein are novel anti-B7-H4 antibodies and methods of their use diagnostically and therapeutically. In one aspect, In this regard, provide herein is an isolated antibody comprising a heavy chain (HC) immunoglobulin variable domain sequence and a light chain (LC) immunoglobulin variable domain sequence, wherein the antibody binds to an epitope of human B7-H4 comprising the amino acid sequence:

IGEDGILSCTFEPDIKLSDIVIQWLKEGVLGLVHEFKEGKDELSEQDEMFRGRT AVFADQVIVGNASLRLKNVQLTDAGTYKCYIITSKGKGNANLEYKTGAFSMPEVNV DYNASSETLRCEAPRWFPQPTVVWASQVDQGANFSEVSNTSFELNSENVTMKVVSV LYNVTINNTYSCMIENDIAKATGDIKVTESEIKRRSHLQLLNSKA or an equivalent thereof.

HLA-G is is a non-classical MHC class I molecule which primarily serves to suppress cytotoxic immune cell function, particularly as a ligand for the inhibitory NK cell receptors.

For instance, in one aspect, disclosed herein are novel anti-HLA-G antibodies and methods of their use diagnostically and therapeutically. In one aspect, In this regard, provide herein is an isolated antibody comprising a heavy chain (HC) immunoglobulin variable domain sequence and a light chain (LC) immunoglobulin variable domain sequence, wherein the antibody binds to an epitope of human HLA-G comprising the amino acid sequence:

GSHSMRYFSA AVSRPGRGEP RFIAMGYVDD TQFVRFDSDS ACPRMEPRAP WVEQEGPEYW EEETRNTKAH AQTDRMNLQT LRGYYNQSEA SSHTLQWMIG CDLGSDGRLL RGYEQYAYDG KDYLALNEDL RSWTAADTAA QISKRKCEAA NVAEQRRAYL EGTCVEWHLA-G YLENGKEMLQ RADPPKTHVT HHPVFDYEAT LRCWALGFYP AEIILTWQRD GEDQTQDVEL VETRPAGDGT FQKWAAVVVP SGEEQRYTCH VQHEGLPEPL MLRWKQSSLP TIPIMGI VAGLVVLAAV VTGAAVAAVL WRKKSSD, or an equivalent thereof.

Lym-1 and Lym-2 are directed against MHC class II HLA-DR molecules which are primarily expressed on the surface of human B cells, dendritic cells, and B-cell derived lymphomas and leukemias. Aspects of the disclosure relate to an isolated nucleic acid sequence encoding a Lym1 or Lym-2 CARs, antibodies, and vectors comprising the isolated nucleic acid sequences.

This disclosure provides a new target for the treatment of solid tumors that include, but are not limited to, ovarian, breast, renal, and prostate carcinomas as well as a B-cell lymphoma or leukemia. The targets, which include LHR, B7-H4, HLA-G, and HLA-DR are often expressed on the majority of these tumors but has restricted off-target positivity and therefore a desirable safety profile. Thus, in one aspect, the compositions are particularly useful in the treatment of tumors or cancerous cell that express or overexpress LHR, B7-H4, HLA-G, HLA-DR.

In one aspect, the antibodies possess a specific binding affinity of at least 10⁻⁶M. In certain aspects, antibodies bind with affinities of at least about 10⁻⁷M, and preferably 10⁻⁸ M, 10⁻⁹M, 10⁻¹⁰M, 10⁻¹¹M, or 10⁻¹²M.

In one aspect, the present disclosure provides an isolated antibodies, the antibodies comprising a heavy chain (HC) immunoglobulin variable domain sequence and a light chain (LC) immunoglobulin variable domain sequence, wherein the antibody binds to an epitope of a luteinizing hormone receptor (LHR), B7-H4, HLA-G, or HLA-DR. In a further aspect, this disclosure provides an isolated anti-LHR, anti-B7-H4, anti-HLA-g, or anti-HLA-DR antibodies or fragments thereof as disclosed herein and a detectable or purification label, alone or in combination with an LHR, B7-H4, HLA-G, or HLA-DR antigen or fragment thereof. Further provided herein is an ex vivo cell comprising this antigen/antibody complex.

Aspects of the disclosure relate to a chimeric antigen receptor (CAR) comprising: (a) an antigen binding domain of an LHR, B7-H4, HLA-G, or HLA-DR antibody; (b) a hinge domain; (c) a transmembrane domain; and (d) an intracellular domain. Further aspects of the disclosure relate to a chimeric antigen receptor (CAR) comprising: (a) an antigen binding domain of a LHR, B7-H4, HLA-G, or HLA-DR antibody; (b) a hinge domain; (c) a CD28 transmembrane domain; (d) one or more costimulatory regions selected from a CD28 costimulatory signaling region, a 4-1BB costimulatory signaling region, an ICOS costimulatory signaling region, and an OX40 costimulatory region; and (e) a CD3 zeta signaling domain and alternatives thereof.

In a further aspect, the present disclosure provides a chimeric antigen receptor (CAR) comprising: (a) an antigen binding domain of an anti-luteinizing hormone receptor (“LHR”), B7-H4, HLA-G, or HLA-DR antibody, (b) a CD8 α hinge domain; (c) a CD8 α transmembrane domain; (d) a CD28 and/or a 4-1BB costimulatory signaling region; and (e) a CD3 zeta signaling domain and alternatives thereof.

In another aspect, the present disclosure provides an isolated nucleic acid sequence encoding the anti-LHR, -B7-H4, -HLA-G, or HLA-DR antibody, or the anti-LHR, -B7-H4, -HLA-G, or -HLA-DR CAR.

In another aspect, the present disclosure provides a vector comprising the isolated nucleic acid sequence encoding the anti-LHR, -B7-H4, -HLA-G, or -HLA-DR antibody, or the anti-LHR, -B7-H4, -HLA-G, or -HLA-DR CAR.

In another aspect, the present disclosure provides a vector comprising the isolated nucleic acid sequence encoding the anti-LHR, -B7-H4, -HLA-G, or -HLA-DR antibody, or the anti-LHR, -B7-H4, -HLA-G, or -HLA-DR CAR.

In another aspect, the present disclosure provides a composition comprising a carrier and one or more of: the anti-LHR, -B7-H4, -HLA-G, or -HLA-DR antibody; and/or the anti-LHR, -B7-H4, -HLA-G, or -HLA-DR CAR; and/or the isolated nucleic acid encoding the anti-LHR, -B7-H4, -HLA-G, or -HLA-DR antibody or the anti-LHR, -B7-H4, -HLA-G, or -HLA-DR CAR; and/or the vector comprising the isolated nucleic acid sequence encoding the anti-LHR, -B7-H4, -HLA-G, or -HLA-DR antibody, or the anti-LHR, -B7-H4, -HLA-G, or -HLA-DR CAR; and/or an isolated cell comprising the anti-LHR, -B7-H4, -HLA-G, or -HLA-DR CAR.

Other aspects of the disclosure relate to an isolated cell comprising a LHR, B7-H4, HLA-G, or HLA-DR CAR and methods of producing such cells. Still other method aspects of the disclosure relate to methods for inhibiting the growth of a tumor, e.g., a solid tumor, and treating a cancer patient comprising administering an effective amount of the isolated cell.

In one aspect, the disclosure provides a composition comprising, or alternatively consisting essentially of, or yet further consisting of a carrier and one or more of: an antibody or fragment thereof, a nucleic acid encoding the antibody or fragment thereof, an isolated cell comprising an anti-LHR, -B7-H4, -HLA-G, or -HLA-DR CAR; and/or the isolated nucleic acid encoding the CAR; and/or the vector comprising the nucleic acid encoding the CAR; and/or the isolated cell expressing an anti-LHR CAR, -B7-H4, -HLA-G, or -HLA-DR; and/or the anti-LHR, -B7-H4, -HLA-G, or -HLA-DR antibody.

### BRIEF DESCRIPTION OF THE DRAWINGS

FIGS. 1A-1C show flow cytometry profiles of (FIG. 1A) LHR on TOV21G, (FIG. 1B) mesothelin on SKOV3, and (FIG. 1C) MUC16 on CAOV3 cell lines.

FIGS. 2A-2C show positive immunohistochemistry staining patterns of (FIG. 2A) LHR antibody on a Stage 2 serous papillary adenocarcinoma; (FIG. 2B) MUC16 antibody on a Stage IIIC endometrioid adenocarcinoma; and (FIG. 2C) mesothelin antibody on a Stage 1C serous papillary adenocarcinoma.

FIG. 3 shows the sequence used to generate LHR-Fc. Amino acid structure of LHR G-protein showing sequence (outlined area) used to generate a LHR-Fc used in immunization and screening methods to identify potential LHR binding antibodies useful for the generation of LHR CARs.

FIG. 4 shows typical flow cytometry screen of LHR-Fc ELISA positive antibodies on the ES-2 ovarian carcinoma cell line demonstrating strong reactivity by hybridoma 8B7 only.

FIG. 5 shows flow cytometry of 5 candidate LHR antibody subclones with highest MFI values on ES-2 human ovarian carcinoma cells.

FIG. 6 shows a schematic diagram of the DNA sequence for, and the theoretical structure of an anti-LHR CAR in the plasma membrane.

FIG. 7 shows the alignments of the heavy chain and light chain sequences of LHR antibody subclones.

FIGS. 8A-D shows a distribution of LHR positive cancers(FIG. 8A); the distribution of LHR intensity with multiple tumor histology groups (FIG. 8B); LHR staining intensity in patients with ovarian, peritoneal, or fallopian tube cancer (FIG. 8C); and LHR staining intensity by tumor pathologic stage group (FIG. 8D).

FIGS. 9A-D LHR expression in prostate cancer, in histology (FIG. 9A), relative mRNA levels in (AD) prostate cancer and castration resistant (CR) prostate cancer (FIG. 9B) and Western blot (FIG. 9C-D).

FIG. 10 shows the backbone of the gene transfer vector is an HIV-based, bicistronic lentiviral vector, pLVX-IRES-ZsGreen containing HIV-1 5′ and 3′ long terminal repeats (LTRs), packaging signal (Ψ), EF1α promoter, internal ribosome entry site (IRES), ZsGreen, a green fluorescent protein, woodchuck hepatitis virus post-transcriptional regulatory element (WPRE), and simian virus 40 origin (SV40). Constitutive expression of the transgene comprising of a scFV specific to LHR, a CD8 hinge and transmembrane region and CD28, 4-1BB and CD3ζ signaling domain, is insured by the presence of the EF-1α promoter. Expression of the detection protein, ZsGreen is carried out by the IRES region. Integration of the vector was assayed by the presence of ZsGreen in the cells, via fluorescent microscopy.

FIG. 11 depicts the results of the cytotoxicity assay of LHR CAR T-cells. Cytotoxicity of the LHR CAR expressing T-cells was determined using an LDH cytotoxicity kit as described in the Methods. Prior to the assay, T-cells were activated using αCD3/CD8 beads (Stem Cell Technologies, 30 ul to 2 ml of media). The activated T-cells were transduced with LHR lentiviral particles, following which the T cells were activated for using the αCD3/CD8 beds. Un-transduced, activated T-cells were used as a control. 3,000 SKOV3 cells were plated per well. LHR transduced T cells were added in ratios of 20:1, 10:1, 5:1 and 1:1 (60,000-3000) to the wells. Each data point represents the average of triplicate measurements.

FIG. 12 depicts mRNA expression of the LHR CAR in primary T-cells. Primary T-cells transduced with the LHR CAR show expression of the LHR mRNA. Primers used spanned the area between the CD8 hinge and the 4-1BB signaling domain (300 bp).

FIGS. 13A-13C show a schematic diagram and HPLC Analysis of Human B7-H4-Fc Fusion Protein Used as Antigen. (FIG. 1A) The vector used to construct the gene; (FIG. 1B) the completed B7-H4-Fc fusion protein in which the B7-H4 was fused to the N-terminus of the immunoglobulin Fc region of human IgG1 producing a dimeric protein used as antigen. (FIG. 1C) HPLC analysis of purified B7-H4-Fc showing the expected retention time indicative of its molecular weight.

FIG. 14 shows representative flow cytometry data for mouse monoclonal anti-human B7-H4 on SKBR-3, HT-29, JAR, and T47D cell lines derived from breast adenocarcinoma, colorectal adenocarcinoma, choriocarcinoma, and breast ductal carcinoma, respectively. Darker line represents cells stained for B7-H4, and lighter line represents cells stained with isotype control. A sheep anti-mouse IgG conjugated to FITC was used as the secondary antibody. Cell surface expression of B7-H4 matches q-PCR data for b7-h4 expression in these cell lines (data not shown).

FIG. 15 shows flow cytometry screening data of newly generated and purified monoclonal antibodies to human B7-H4. Subclones of positive hybridomas (35-8 and 5F6-6) were selected for the generation of CAR T-cells based upon these results. Clone 35-8 was then sequenced and used to produce B7-H4 CAR T-cells for immunotherapy.

FIGS. 16A-B show representative images of B7-H4 antibody (clone #35-8) staining on 16normal and cancer tissue microarrays. (FIG. 16A) B7-H4 staining on normal tissues. (FIG. 16B) B7-H4 staining on normal and cancer tissue of the breast. Other normal tissues found negative for B7-H4 positivity (not shown) include the following: adrenal gland, bone marrow, cerebellum, esophagus, hypophysis, intestine, lymph node, ovary, prostate, stomach, testis, thyroid, thymus, tongue, uterine, skin, and nerve tissue.

FIG. 17 shows a schematic diagram of the DNA sequence for, and the theoretical structure of third generation anti-B7-H4 CAR in the plasma membrane.

FIG. 18A-B shows immunohistochemistry staining of B7-H4 on sections of (FIG. 18A) human breast carcinoma biopsy and (FIG. 18B) SKBR3 human breast cancer cell line pellet showing cell surface positivity for antigen (brown staining).

FIG. 19 shows a schematic representation of the gene transfer vector and of the transgene. The backbone of the gene transfer vector is an HIV-based, bicistronic lentiviral vector, pLVX-IRES-ZsGreen containing HIV-1 5′ and 3′ long terminal repeats (LTRs), packaging signal (Ψ), EF1α promoter, internal ribosome entry site (IRES), ZsGreen, a green fluorescent protein, woodchuck hepatitis virus post-transcriptional regulatory element (WPRE), and simian virus 40 origin (SV40). Constitutive expression of the transgene comprising of a scFV specific to B7-H4, a CD8 hinge and transmembrane region and CD28, 4-1BB and CD3ζ signaling domain, is insured by the presence of the EF-1α promoter. Expression of the detection protein, ZsGreen is carried out by the IRES region. Integration of the vector can be assayed by the presence of ZsGreen in the cells, via fluorescent microscopy

FIG. 20 shows cytotoxicity of the B7-H4 CAR T-cells. Cytotoxicity of the B7-H4 CAR expressing T-cells was determined using an LDH cytotoxicity kit as described in the Methods. Prior to the assay, T-cells were activated using αCD3/CD8 beads (Stem Cell Technologies, 30 μl to 2 ml of media). The activated T-cells were transduced with B7-H4 lentiviral particles, following which the T cells were activated for using the αCD3/CD8 beads. Un-transduced, activated T-cells were used as a control. 3000 SKBR3 cells were plated per well. B7-H4 transduced T cells were added in ratios of 20:1, 10:1, 5:1 and 1:1 (60,000-3000 cells) to the wells. Each data point represents the average of triplicate measurements.

FIG. 21 shows flow cytometry screening data of newly generated monoclonal antibodies to human HLA-G. Subclones of positive hybridomas (3H11-12 and 4E3-1) were selected for the generation of CAR T-cells based upon these results.

FIGS. 22A-22D show immunohistochemistry of HLA-G reactivity in papillary thyroid cancer and normal thyroid tissue with HLA-ABC control staining. FIG. 22A shows low magnification of HLA-G positive papillary thyroid carcinoma section using antibody 4E3-1 (100×). FIG. 22B shows higher magnification of second papillary thyroid carcinoma positive for HLA-G (250×). FIG. 22C shows negative reactivity of normal thyroid tissues for HLA-G (250×), and FIG. 22D shows positive reactivity of normal thyroid tissue for HLA-ABC (100×).

FIG. 23 shows schematic diagram of the DNA sequence for, and the theoretical structure of third generation anti-HLA-G CAR in the plasma membrane.

FIG. 24 shows additional antibody screening, as described in FIG. 1.

FIG. 25 depicts a schematic of the gene-transfer vector and the transgene. The backbone of the gene transfer vector is an HIV-based, bicistronic lentiviral vector, pLVX-IRES-ZsGreen containing HIV-1 5′ and 3′ long terminal repeats (LTRs), packaging signal (Ψ), EF1α promoter, internal ribosome entry site (IRES), ZsGreen, a green fluorescent protein, woodchuck hepatitis virus post-transcriptional regulatory element (WPRE), and simian virus 40 origin (SV40). Constitutive expression of the transgene comprising of a scFV specific to HLA-G, a CD8 hinge and transmembrane region and CD28, 4-1BB and CD3ζ signaling domain, is insured by the presence of the EF-1α promoter. Expression of the detection protein, ZsGreen is carried out by the IRES region. Integration of the vector can be assayed by the presence of ZsGreen in the cells, via fluorescent microscopy.

FIG. 26 shows cytotoxicity of the HLA-G CAR T-cells. Cytotoxicity of the HLA-G CAR expressing T-cells was determined using an LDH cytotoxicity kit as described in the Methods. Prior to the assay, T-cells were activated using αCD3/CD8 beads (Stem Cell Technologies, 30 ul to 2 ml of media). The activated T-cells were transduced with HLA-G lentiviral particles, following which the T cells were activated for using the αCD3/CD8 beads. Un-transduced, activated T-cells and the TLBR-2 T lymphoma cell line were used as controls. 3,000 SKOV3 or TLBR-2 cells were plated per well. HLA-G transduced T cells were added in ratios of 20:1, 10:1, 5:1 and 1:1 (60,000-3000 cells) to the wells. Each data point represents the average of triplicate measurements.

FIG. 27 shows protein expression of the HLA-G CAR. T-cells transduced with the HLA-G CAR lentiviral particles express protein for the HLA-G CAR. The estimated size of the CAR protein is 60 kDa. A CD3ζ antibody was used to detect the protein. Fifty μg of protein was used for the western blot. β-actin was used as a loading control.

FIGS. 28A-28F show flow cytometric analysis of (FIG. 28A) negative control; (FIG. 28B) Lym-1; (FIG. 28C) Lym-1 and B1; (FIG. 28D) B1 only; (FIG. 28E) Lym-2; and (FIG. 28F) Lym-2 and B1 staining reactivity with normal peripheral blood lymphocytes of patients. Both Lym-1 and Lym-2 have different profiles of binding to normal human peripheral B cells.

FIGS. 29A-29B show Lym-1 and Lym-2 staining of normal human tonsil demonstrating membrane positivity in B-cell germinal centers. Differences in staining patterns are evident between Lym-1 (FIG. 29A) and Lym-2 (FIG. 29B). Only scattered interfollicular dendritic cells are positive for both antibodies in the T-cell zones (IHC, frozen sections, ×325).

FIGS. 30A and 30B show immunoperoxidase staining of Lym-1 and Lym-2 monoclonal antibodies with an intermediate grade malignant B-cell lymphoma. Immunoperoxidase staining of Lym-1 (FIG. 30A) and Lym-2 (FIG. 30B) monoclonal antibodies with an intermediate grade malignant B-cell lymphoma (frozen sections, ×720). Note prominent membrane staining pattern of majority of cells in the section.

FIGS. 31A-31C show binding profiles and Scatchard Plots of (FIG. 31A) Binding profiles of Lym-1 monoclonal antibodies to Raji cells and Lym-2 monoclonal antibodies to ARH-77 cells; (FIG. 31B) Scatchard plot analysis of Lym-1 monoclonal antibodies with Raji cells; (FIG. 31C) Scatchard plot analysis of Lym-2 monoclonal antibodies with ARH-77 cells.

FIGS. 32A and 32B show immunoprecipitation of ³⁵S-methionine and ¹⁴C-leucine-labeled Raji proteins by Lym-1 (FIG. 32A) and SC-2 anti-HLA-DR antibody (FIG. 32B).

FIGS. 33A and 33B show a construction schematic of (FIG. 33A) Lym-1 and (FIG. 33B) Lym-2 CAR T-cells for immunotherapy. FIGS. 6A and 6B disclose a flexible linker sequence.

FIG. 34 depicts a schematic a non-limiting exemplary Lym-1 gene-transfer vector and transgene. The backbone of the gene transfer vector is an HIV-based, bicistronic lentiviral vector, pLVX-IRES-ZsGreen containing HIV-1 5′ and 3′ long terminal repeats (LTRs), packaging signal (Ψ), EF1α promoter, internal ribosome entry site (IRES), ZsGreen, a green fluorescent protein, woodchuck hepatitis virus post-transcriptional regulatory element (WPRE), and simian virus 40 origin (SV40).Constitutive expression of the transgene comprising of αCD8 leader sequence, a scFV specific to Lym-1, a CD8 hinge and transmembrane region and 4-1BB and CD3ζ signaling domain, is insured by the presence of the EF-1α promoter. Expression of the detection protein, ZsGreen is carried out by the IRES region. Integration of the vector can be assayed by the presence of ZsGreen in the cells, via fluorescent microscopy.

FIG. 35 shows expression of Lym-1 CAR on primary human T-cells. T-cells were transduced with the Lym-1 CAR and stained with Biotein-Protein L, followed by Streptavidin-PE. Cells were analyzed by flow cytometry.

FIG. 36 shows cytotoxicity of the Lym-1-CAR T-cells. Cytotoxicity of the Lym-1 CAR expressing T-cells was determined using an LDH cytotoxicity kit as described in the Methods. Prior to the assay, T-cells were activated using αCD3/CD8 beads (Stem Cell Technologies, 30 ul to 2 ml of media). The activated T-cells were transduced with Lym-1 CAR lentiviral particles, following which the T cells were activated using the αCD3/CD8 beads. Un-transduced, activated T-cells were used as a control. 15,000 Raji cells were plated per well. Lym-1 CAR transduced T cells were added in ratios of 20:1, 10:1, 5:1 and 1:1 to the wells. Each data point represents the average of triplicate measurements.

FIG. 37 depicts a schematic a non-limiting exemplary Lym-2 gene-transfer vector and transgene. The backbone of the gene transfer vector is an HIV-based, bicistronic lentiviral vector, pLVX-IRES-ZsGreen containing HIV-1 5′ and 3′ long terminal repeats (LTRs), packaging signal (Ψ), EF1α promoter, internal ribosome entry site (IRES), ZsGreen, a green fluorescent protein, woodchuck hepatitis virus post-transcriptional regulatory element (WPRE), and simian virus 40 origin (SV40).Constitutive expression of the transgene comprising of a CD8 leader sequence, an scFV specific to Lym-2, a CD8 hinge and transmembrane region and CD28, 4-1BB and CD3ζ signaling domain, is insured by the presence of the EF-1α promoter. Expression of the detection protein, ZsGreen is carried out by the IRES region. Integration of the vector can be assayed by the presence of ZsGreen in the cells, via fluorescent microscopy.

FIG. 38 shows expression of Lym-2 CAR on primary human T-cells. T-cells were transduced with the Lym-2 CAR and stained with Biotein-Protein L, followed by Streptavidin-PE. Cells were analyzed by flow cytometry.

FIG. 39 shows cytotoxicity of the Lym-2-CAR T-cells. Cytotoxicity of the Lym-2 CAR expressing T-cells was determined using an LDH cytotoxicity kit as described in the Methods. Prior to the assay, T-cells were activated using αCD3/CD8 beads (Stem Cell Technologies, 30 ul to 2 ml of media). The activated T-cells were transduced with Lym-2 CAR lentiviral particles, following which the T cells were activated using the αCD3/CD8 beads. Un-transduced, activated T-cells were used as a control. 15,000 Raji cells were plated per well. Lym-2 CAR transduced T cells were added in ratios of 20:1, 10:1, 5:1 and 1:1 to the wells. Each data point represents the average of triplicate measurements.

FIG. 40 demonstrates that Lym-1, Lym-2, and CD19 CAR T-cells are highly cytotoxic to human lymphoma Raji cells. Raji Burkitt's lymphoma cells are positive for both HLA-Dr targeted by Lym-1 and Lym-2 and also CD19 which acted as a positive control for CD19 CAR T-cells. Negative controls consisted of CD3+ T cells and Zsgreen cells.

FIG. 41 demonstrates that Lym-1, Lym-2, but not CD19 CAR are highly cytolytic against HLA-Dr positive but CD19 negative TLBR-2 human T lymphoma cells in vitro. TLBR-2 human T-lymphoma cells derived from a breast implant associated lymphoma is positive for HLA-Dr but not CD19 (Lechner et al. (2012) Clin. Cancer Res. 18 (17):4549-4559). These results demonstrate the specificity of the Lym-1 and Lym-2 CAR T-cells and their potency in killing HLA-Dr positive tumors. The percentage of Lym-1 CAR-T and CD19 CAR-T positive cells were adjusted to 50% using regular un-transduced primary T cells. The percentage of Lym-2 CAR-T cells was 24%.

FIG. 42 shows the results of FACs analysis of transfected NK cells.

### DETAILED DESCRIPTION

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³M⁻¹ greater, at least 10⁴M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997. An “antigen binding fragment” of an antibody is a portion of an antibody that retains the ability to specifically bind to the target antigen of the antibody.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies and human antibodies.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds LHR, B7-H4, HLA-G, or HLA-DR will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, the term “antigen binding domain” refers to any protein or polypeptide domain that can specifically bind to an antigen target.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non-limiting exemplary polynucleotide sequences that encode for components of each domain are disclosed herein, e.g.:

Hinge domain: IgG1 heavy chain hinge sequence:

CTCGAGCCCAAATCTTGTGACAAAACTCACACATGCCCACCGTGCCCG

Transmembrane domain: CD28 transmembran region:

TTTTGGGTGCTGGTGGTGGTTGGTGGAGTCCTGGCTTGCTATAGCTTGC
TAGTAACAGTGGCCTTTATTATTTTCTGGGTG

Intracellular domain: 4-1BB co-stimulatory signaling region:

AAACGGGGCAGAAAGAAACTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG

Intracellular domain: CD28 co-stimulatory signaling region:

AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

Intracellular domain: CD3 zeta signaling region:

AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGAT
GTTTTGGACAAGAGACGTGGCCGGGACCCTGAGATGGGGGGAAAGCCGAG
AAGGAAGAACCCTCAGGAAGGCCTGTACAATGAACTGCAGAAAGATAAGA
TGGCGGAGGCCTACAGTGAGATTGGGATGAAAGGCGAGCGCCGGAGGGGC
AAGGGGCACGATGGCCTTTACCAGGGTCTCAGTACAGCCACCAAGGACAC
CTACGACGCCCTTCACATGCAGGCCCTGCCCCCTCGCTAA

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non-limiting examples of such domains are provided herein.

As used herein, the term “HLA-DR” (refers to an MHC class II cell surface receptor associated with this name and any other molecules that have analogous biological function that share at least 80% amino acid sequence identity, preferably 90% sequence identity, or alternatively at least 95% sequence identity with any HLA-DR variant, including but not limited to any one of its several variants, including but not limited to HLA-DR serotypes DR1 to DR 75 comprising a combination of HLA-DRA and HLA-DRB haplotypes. Examples of the HLA-DR sequences are known in the art and non-limited examples of such are disclosed in Rose, L. M. et al. (1996) Cancer Immunol. Immunother. 43:26-30:

HLA-DRB1*1001 [DR10]

GDTRPRFLEEVKFECHFFNGTERVRLLERRVHNQEEYARYDSDVGEYRAVTELGRP DAEWNSQKDLLERRRAAVDTYCRHNYGVGESFTVQRRVQPKVTVYPSKTQPLQH HNLLVCSVNGFYPGSIEVRWFRNGQEEKTGVVSTGLIQNGDWTFQTLVMLETVPQS GEVYTCQVEHPSVMSPLTVEWRARSESAQSKMLSGVGGFVLGLLFLGAGLFIYFRN QKGHSGLPPTGFLS;

HLA-DRB3*0201 [DR52]

GDTRPRFLELLKSECHFFNGTERVRFLERHFHNQEEYARFDSDVGEYRAVFELGRPD AEYWNSQKDLLEQKRGQVDNYCRHNYGVVESFTVQRRVHPQVTVYPAKTQPLQH HNLLVCSVSGFYPGSIEVRWFRNGQEEKAGVVSTGLIQNGDWTFQTLVMLETFPRSG EVYTCQVEHPSVTSPLTVEWSARSESAQSKMLSGVGGFVLGLLFLGAGLFIYFRNQK GHSGLQPTGFLS;

HLA-DRB1*0301 [DR17 (3)]

GDTRPRFLEYSTSECHFFNGTERVRYLDRYFHNQEENVRFDSDVGEFRAVTELGRPD AEWNSQKDLLEQKRGRVDNYCRHNYGVVESFTVQRRVHPKVTVYPSKTQPLQHH NLLVCSVSGFYPGSIEVRWFRNGQEEKTGVVSTGLIQNGDWTFQTLVMLETVPRSGE VYTCQVEHPSVTSPLTVEWRARSESAQSKMLSGVGGFVLGLLFLGAGLFIYFRNQKG HSGLQPRGFLS, as well as equivalents of each thereof.

Rose et al. also discloses an exemplary epitope to which an HLA-DR specific antibody may bind and therefore can serve as an immunogen for the generation of additional antibodies, monoclonal antibodies and antigen binding fragments of each thereof. The sequences associated with each of the listed reference(s) and GenBank Accession Numbers that correspond to the name HLA-DR or its equivalents including but not limited to the specified HLA-DR subtypes are herein incorporated by reference as additional non-limiting examples.

A “composition” typically intends a combination of the active agent, e.g., a CAR T cell or a CAR NK cell, an antibody, a compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

As used herein, the term “luteinizing hormone receptor” (LHR) refers to a specific molecule associated with this name and any other molecules that have analogous biological function that share at least 70%, or alternatively at least 80% amino acid sequence identity, preferably 90% sequence identity, more preferably at least 95% sequence identity with the LHR sequence as shown herein. The protein sequences associated with GenBank Accession Nos. AAB19917.2 (Homo sapiens), or AAA39432.1 (Mus musculus), or AAA41529.1 (Rattus norvegicus) provide additional example sequences of LHR. Non-limiting examples of such include:

Luteinizing hormone receptor [Homo sapiens]:
MKQRFSALQLLKLLLLLQPPLPRALREALCPEPCNCVPDGALRCPGPTAGL
TRLSLAYLPVKVIPSQAFRGLNEVIKIEISQIDSLERIEANAFDNLLNLSE
ILIQNTKNLRYIEPGAFINLPRLKYLSICNTGIRKFPDVTKVFSSESNFIL
EICDNLHITTIPGNAFQGMNNESVTLKLYGNGFEEVQSHAFNGTTLTSLEL
KENVHLEKMEINGAFRGATGPKTLDISSTKLQALPSYGLESIQRLIATSSY
SLKKLPSRETFVNLLEATLTYPSHCCAFRNLPTKEQNFSHSISENFSKQCE
STVRKVNNKTLYSSMLAESELSGWDYEYGFCLPKTPRCAPEPDAFNPCEDI
MGYDFLRVLIWLINILAIMGNMTVLFVLLTSRYKLTVPRFLMCNLSFADFC
MGLYLLLIASVDSQTKGQYYNHAIDWQTGSGCSTAGFFTVFASELSVYTLT
VITLERWHTITYAIHLDQKLRLRHAILIMLGGWLFSSLIAMLPLVGVSNYM
KVSICFPMDVETTLSQVYILTILILNVVAFFIICACYIKIYFAVRNPELMA
TNKDTKIAKKMAILIFTDFTCMAPISFFAISAAFKVPLITVTNSKVLLVLF
YPINSCANPFLYAIFTKTFQRDFFLLLSKFGCCKRRAELYRRKDFSAYTSN
CKNGFTGSNKPSQSTLKLSTLHCQGTALLDKTRYTEC
Luteinizing hormone receptor [Mus musculus]:
MGRRVPALRQLLVLAMLVLKQSQLHSPELSGSRCPEPCDCAPDGALRCPGP
RAGLARLSLTYLPVKVIPSQAFRGLNEVVKIEISQSDSLERIEANAFDNLL
NLSEILIQNTKNLLYIEPGAFTNLPRLKYLSICNTGIRTLPDVSKISSSEF
NFILEICDNLYITTIPGNAFQGMNNESITLKLYGNGFEEVQSHAFNGTTLI
SLELKENIYLEKMHSGTFQGATGPSILDVSSTKLQALPSHGLESIQTLIAT
SSYSLKTLPSREKFTSLLVATLTYPSHCCAFRNLPKKEQNFSFSIFENFSK
QCESTVREANNETLYSAIFEENELSGWDYDYDFCSPKTLQCTPEPDAFNPC
EDIMGYAFLRVLIWLINILAIFGNLTVLFVLLTSRYKLTVPRFLMCNLSFA
DFCMGLYLLLIASVDSQTKGQYYNHAIDWQTGSGCSAAGFFTVFASELSVY
TLTVITLERWHTITYAVQLDQKLRLRHAIPIMLGGWIFSTLMATLPLVGVS
SYMKVSICLPMDVESTLSQVYILSILLLNAVAFVVICACYVRIYFAVQNPE
LTAPNKDTKIAKKMAILIFTDFTCMAPISFFAISAAFKVPLITVTNSKVLL
VLFYPVNSCANPFLYAVFTKAFQRDFFLLLSRFGCCKHRAELYRRKEFSAC
TFNSKNGFPRSSKPSQAALKLSIVHCQQPTPPRVLIQ
Luteinizing hormone receptor [Rattus norvegicus]:
MGRRVPALRQLLVLAVLLLKPSQLQSRELSGSRCPEPCDCAPDGALRCPGP
RAGLARLSLTYLPVKVIPSQAFRGLNEVVKIEISQSDSLERIEANAFDNLL
NLSELLIQNTKNLLYIEPGAFTNLPRLKYLSICNTGIRTLPDVTKISSSEF
NFILEICDNLHITTIPGNAFQGMNNESVTLKLYGNGFEEVQSHAFNGTTLI
SLELKENIYLEKMHSGAFQGATGPSILDISSTKLQALPSHGLESIQTLIAL
SSYSLKTLPSKEKFTSLLVATLTYPSHCCAFRNLPKKEQNFSFSIFENFSK
QCESTVRKADNETLYSAIFEENELSGWDYDYGFCSPKTLQCAPEPDAFNPC
EDIMGYAFLRVLIWLINILAIFGNLTVLFVLLTSRYKLTVPRFLMCNLSFA
DFCMGLYLLLIASVDSQTKGQYYNHAIDWQTGSGCGAAGFFTVFASELSVY
TLTVITLERWHTITYAVQLDQKLRLRHAIPIMLGGWLFSTLIATMPLVGIS
NYMKVSICLPMDVESTLSQVYILSILILNVVAFVVICACYIRIYFAVQNPE
LTAPNKDTKIAKKMAILIFTDFTCMAPISFFAISAAFKVPLITVTNSKILL
VLFYPVNSCANPFLYAIFTKAFQRDFLLLLSRFGCCKRRAELYRRKEFSAY
TSNCKNGFPGASKPSQATLKLSTVHCQQPIPPRALTH

As used herein, the term “B7-H4” (also known as VTCN1, H4, B7h.5, B7S1, B7X, or PRO129) refers to a specific molecule associated with this name and any other molecules that have analogous biological function that share at least 80% amino acid sequence identity, preferably 90% sequence identity, more preferably at least 95% sequence identity with B7-H4. Examples of the B7-H4 sequence are provided herein. In addition, the protein sequences associated with GenBank Accession Nos. AY280973.1 (Mus musculus) and NP_078902 (Homo sapiens) provide example sequences of B7-H4 in various animals; the referenced genes have 87% homology. The sequences associated with each of the listed GenBank Accession Nos. are herein incorporated by reference. As used herein, the term “anti-B7-H4,” in reference to an antibody or receptor, refers to an antibody or receptor that specifically binds to B7-H4 and includes reference to any antibody which is generated against B7-H4.

Provided are novel anti-B7-H4 antibodies and methods of their use diagnostically and therapeutically. In one aspect, In this regard, provide herein is an isolated antibody comprising a heavy chain (HC) immunoglobulin variable domain sequence and a light chain (LC) immunoglobulin variable domain sequence, wherein the antibody binds to an epitope of human B7-H4 comprising the amino acid sequence:

IGEDGILSCTFEPDIKLSDIVIQWLKEGVLGLVHEFKEGKDELSEQDEMFRGRT AVFADQVIVGNASLRLKNVQLTDAGTYKCYIITSKGKGNANLEYKTGAFSMPEVNV DYNASSETLRCEAPRWFPQPTVVWASQVDQGANFSEVSNTSFELNSENVTMKVVSV LYNVTINNTYSCMIENDIAKATGDIKVTESEIKRRSHLQLLNSKA or an equivalent thereof.

In certain embodiments disclosed herein, the antibody comprises a heavy chain (HC) immunoglobulin variable domain sequence and a light chain (LC) immunoglobulin variable domain sequence, wherein the antibody binds to an epitope of human B7-H4 comprising, or alternatively consisting essentially of, or yet further consisting of, an amino acid sequence wherein the HC comprises any one of the following a HC CDRH1 comprising the amino acid sequence GFTFSSFG, GFTFSSYG, or GYTFTDY; and/or a HC CDRH2 comprising the amino acid sequence ISSGSSTL, ISSSNSTI, or INPNNGGT; and/or a HC CDRH3 comprising the amino acid sequence ARPLYYYGSVMDY or RPYYYGSSYDY.

In certain embodiments disclosed herein, the antibody comprises a heavy chain (HC) immunoglobulin variable domain sequence and a light chain (LC) immunoglobulin variable domain sequence, wherein the antibody binds to an epitope of human B7-H4 comprising, or alternatively consisting essentially of, or yet further consisting of, an amino acid sequence wherein the LC comprises a LC CDRL1 comprising the amino acid QSIVHRNGNTY, QSIVHSNGNTY, or ENIGSY; and/or a LC CDRL2 comprising the amino acid sequence KVS or AAT; and/or a LC CDRL3 comprising the amino acid sequence FQGSYVPPT, FQGSHVPLT, QHYYSTLVT.

As used herein, the term “HLA-G” (also known as B2 Microglobulin or MHC-G) refers to a specific molecule associated with this name and any other molecules that have analogous biological function that share at least 80% amino acid sequence identity, preferably 90% sequence identity, more preferably at least 95% sequence identity with HLA-G, including but not limited to any one of its several isoforms, including by not limited to membrane-bound isoforms (e.g., HLA-G1, HLA-G2, HLA-G3, HLA-G4), soluble isoforms (e.g., HLA-G5, HLA-G6, HLA-G7), and soluble forms generated by proteolytic cleavage of membrane-bound isoforms (e.g. sHLA-G1). Examples of the HLA-G sequence are provided herein. In addition, the protein sequences associated with GenBan Accession Nos. are exemplary: NM_002127.5 XM_006715080.1 XM_006725041.1 XM_006725700.1 XM_0067259091 An example is NM_002127.5 Sequence:

MVVMAPRTLFLLLSGALTLTETWAGSHSMRYFSAAVSRPGRGEPRFIAMG
YVDDTQFVRFDSDSACPRMEPRAPWVEQEGPEYWEEETRNTKAHAQTDRM
NLQTLRGYYNQSEASSHTLQWMIGCDLGSDGRLLRGYEQYAYDGKDYLAL
NEDLRSWTAADTAAQISKRKCEAANVAEQRRAYLEGTCVEWLHRYLENGK
EMLQRADPPKTHVTHHPVFDYEATLRCWALGEYPAEIILTWQRDGEDQTQ
DVELVETRPAGDGTFQKWAAVVVPSGEEQRYTCHVQHEGLPEPLMLRWKQ
SSLPTIPIMGIVAGLVVLAAVVTGAAVAAVLWRKKSSD

The sequences associated with each of the above listed GenBank Accession Nos. are herein incorporated by reference.

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. Non-limiting examples of such include:

Human CD8 alpha hinge domain:

PAKPTTTPAPRPPTPAPTIASQPLSLRPEACRPAAGGAVHTRGLDFACDI
Y

Mouse CD8 alpha hinge domain:

KVNSTTTKPVLRTPSPVHPTGTSQPQRPEDCRPRGSVKGTGLDFACDIY

Cat CD8 alpha hinge domain:

PVKPTTTPAPRPPTQAPITTSQRVSLRPGTCQPSAGSTVEASGLDLSCDI
Y

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (NCBI Reference Sequence: NP_001759.3), or the amino acid positions 197 to 217 of the mouse T-cell surface glycoprotein CD8 alpha chain (NCBI Reference Sequence: NP_001074579.1), and the amino acid positions190 to 210 of the rat T-cell surface glycoprotein CD8 alpha chain (NCBI Reference Sequence: NP_113726.1) provide additional example sequences of the CD8 α transmembrane domain. The sequences associated with each of the listed NCBI are provided as follows:

Human CD8 alpha transmembrane domain:

IYIWAPLAGTCGVLLLSLVIT

Mouse CD8 alpha transmembrane domain:

IWAPLAGICVALLLSLIITLI

Rat CD8 alpha transmembrane domain:

IWAPLAGICAVLLLSLVITLI

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain. The sequences associated with each of the listed accession numbers are incorporated herein.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. The example sequence of the 4-1BB costimulatory signaling region is provided in U.S. application Ser. No. 13/826,258. The sequence of the 4-1BB costimulatory signaling region associated disclosed in the U.S. application Ser. No. 13/826,258 is listed as follows:

The 4-1BB costimulatory signaling region:

KRGRKKLLYIFKQPFMRPVQTTQEEDGCSCRFPEEEEGGCEL

As used herein, the term “CD28 costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the CD28 costimulatory signaling region sequence shown herein. Exemplary CD28 costimulatory signaling domains are provided in U.S. Pat. No. 5,686,281; Geiger, T. L. et al., Blood 98: 2364-2371 (2001); Hombach, A. et al., J Immunol 167: 6123-6131 (2001); Maher, J. et al. Nat Biotechnol 20: 70-75 (2002); Haynes, N. M. et al., J Immunol 169: 5780-5786 (2002); Haynes, N. M. et al., Blood 100: 3155-3163 (2002). Non-limiting examples include residues 114-220 of the below CD28 Sequence: MLRLLLALNL FPSIQVTGNK ILVKQSPMLV AYDNAVNLSC KYSYNLFSRE FRASLHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS, and equivalents thereof.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Publication 2015/0017141A1 the exemplary polynucleotide sequence provided below.

ICOS costimulatory signaling region:

ACAAAAAAGA AGTATTCATC CAGTGTGCAC GACCCTAACG
GTGAATACAT GTTCATGAGA GCAGTGAACA CAGCCAAAAA
ATCCAGACTC ACAGATGTGA CCCTA

As used herein, the term “OX40 costimulatory signaling region” refers to a specific protein fragment associated with this name and any other molecules that have analogous biological function that share at least 70%, or alternatively at least 80% amino acid sequence identity, or alternativley 90% sequence identity, or alternatively at least 95% sequence identity with the OX40 costimulatory signaling region sequence as shown herein. Non-limiting example sequences of the OX40 costimulatory signaling region are disclosed in U.S. Publication 2012/20148552A1, and include the exemplary sequence provided below.

OX40 costimulatory signaling region:

AGGGACCAG AGGCTGCCCC CCGATGCCCA CAAGCCCCCT
GGGGGAGGCA GTTTCCGGAC CCCCATCCAA GAGGAGCAGG
CCGACGCCCA CTCCACCCTG GCCAAGATC

As used herein, the term “CD3 zeta signaling domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD3 zeta signaling domain sequence as shown herein. The example sequences of the CD3 zeta signaling domain are provided in U.S. Pub. No. US 2013/0266551A1. The sequence associated with the CD3 zeta signaling domain is listed as follows:

The CD3 zeta signaling domain:

RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™) EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4,-5,-6,-7,-8,-9,-10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (www.atcc.org/) and the German Collection of Microorganisms and Cell Cultures (https://www.dsmz.de/).

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term signal peptide or signal polypeptide intends an amino acid sequence usually present at the N-terminal end of newly synthesized secretory or membrane polypeptides or proteins. It acts to direct the polypeptide across or into a cell membrane and is then subsequently removed. Examples of such are well known in the art. Non-limiting examples are those described in U.S. Pat. Nos. 8,853,381 and 5,958,736.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “isolated cell” generally refers to a cell that is substantially separated from other cells of a tissue. “Immune cells” includes, e.g., white blood cells (leukocytes) which are derived from hematopoietic stem cells (HSC) produced in the bone marrow, lymphocytes (T cells, B cells, natural killer (NK) cells), myeloid-derived cells (neutrophil, eosinophil, basophil, monocyte, macrophage, dendritic cells), as well as precursors thereof committed to immune lineages. Precursors of T-cells are lineage restricted stem and progenitor cells capable of differentiating to produce a mature T-cell. Precursors of T-cells include HSCs, long term HSCs, short term HSCs, multipotent progenitor cells (MPPs), lymphoid primed multipotent progenitor cells (LMPPs), early lymphoid progenitor cells (ELPs), common lymphoid progenitor cells (CLPs), Pro-T-cells (ProT), early T-lineage progenitors/double negative 1 cells (ETPs/DN1), double negative (DN) 2a, DN2b, DN3a, DN3b, DN4, and double positive (DP) cells. Markers of such T-cell precursors in humans include but are not limited to: HSCs: CD34+ and, optionally, CD38−; long term HSCs: CD34+ CD38− and lineage negative, wherein lineage negative means negative for one or more lineage specific markers selected from the group of TER119, Mac1, Gr1, CD45R/B220, CD3, CD4, and CD8; MPPs: CD34+ CD38− CD45RA− CD90− and, optionally, lineage negative; CLP: CD34+ CD38+ CD10+ and, optionally, lineage negative; LMPP/ELP: CD45RA+ CD62L+ CD38− and, optionally, lineage negative; DN1: CD117− CD34+ CD38− CD1a−; DN2: CD117+ CD34+ CD38+ CD1a−; DN3: CD34+ CD38+ CD1a+; DN4: CD4+ CD3−; DP: CD4+ CD8+ and, optionally, CD3+. Precursors of NK cells are lineage restricted stem and progenitor cells capable of differentiating to produce a mature NK cell. NK precursors include HSCs, long term HSCs, short term HSCs, multipotent progenitor cells (MPPs), common myeloid progenitors (CMP), granulocyte-macrophage progenitors (GMP), pro-NK, pre-NK, and immature NK (iNK). Markers of such NK precursors include but are not limited to: CMP: CD56− CD36− CD33+ CD34+ NKG2D− NKp46−; GMP: CD56− CD36− CD33+ CD34+ NKG2D− NKp46−; pro-NK: CD34+ CD45RA+ CD10+ CD117− CD161−; pre-NK: CD34+ CD45RA+ CD10− CD117+ CD161+/−; and iNK: CD34− CD117+ CD161+ NKp46− CD94/NKG2A−. In some aspects, markers of NK cell precursors include but are not limited to CD117+ CD161+ CD244+ CD33+ CD56− NCR− CD94/NKG2A− and LFA-1−. Phenotyping reagents to detect precursor cell surface markers are available from, for example, BD Biosciences (San Jose, Calif.) and BioLegend (San Diego, Calif.). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “CRISPR” refers to a technique of sequence specific genetic manipulation relying on the clustered regularly interspaced short palindromic repeats pathway (CRISPR). CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA.

The term “guide RNA” or “gRNA” as used herein refers to the guide RNA sequences used to target the CRISPR complex to a specific nucleotide sequence such as a specific region of a cell's genome. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83).

The term “inhibitory RNA” refers to an RNA molecule capable of RNA interference, a mechanism whereby an inhibitory RNA molecule targets a messenger RNA (mRNA) molecule, resulting in inhibition gene expression and/or translation. RNA interference is also known as post-transcriptional gene silencing. Exemplary inhibitory RNAs include but are not limited to antisense RNAs, microRNAs (miRNA), small interfering RNAs (siRNA), short hairpin RNAs (shRNA), double stranded RNA (dsRNA) and intermediates thereof. Methods of designing, cloning, and expressing inhibitory RNAs are known in the art (e.g. McIntyre et al, BMC Biotechnol 2006; 6:1; Moore et al. Methods Mol Biol. 2010; 629: 141-158) and custom RNAi kits are commercially available (e.g. GeneAssist™ Custom siRNA Builder, ThermoFisher Scientific, Waltham, Mass.).

As used herein, the term “autologous,” in reference to cells refers to cells that are isolated and infused back into the same subject (recipient or host). “Allogeneic” refers to non-autologous cells.

An “ effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas.

The term “ovarian cancer” refers to a type of cancer that forms in issues of the ovary, and has undergone a malignant transformation that makes the cells within the cancer pathological to the host organism with the ability to invade or spread to other parts of the body. The ovarian cancer herein comprises type I cancers of low histological grade and type II cancer of higher histological grade. Particularly, the ovarian cancer includes but is not limited to epithelial carcinoma, serous carcinoma, clear-cell carcinoma, sex cord stromal tumor, germ cell tumor, dysgerminoma, mixed tumors, secondary ovarian cancer, low malignant potential tumors.

The term “prostate cancer” refers to a type of cancer that develops in the prostate, a gland in the male reproductive system. The prostate cancer herein includes but is not limited to adenocarcinoma, sarcomas, small cell carcinomas, neuroendocrine tumors, transitional cell carcinomas.

The term “B cell lymphoma or leukemia” refers to a type of cancer that forms in issues of the lymphatic system or bone marrow, and has undergone a malignant transformation that makes the cells within the cancer pathological to the host organism with the ability to invade or spread to other parts of the body.

The term “thyroid cancer” refers to a type of cancer that develops in the thyroid.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³² P, ³⁵ S or ¹²⁵ I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, the term “switch” refers to a mechanism by which the expression, activation, or stability of a CAR or a component of a CAR is controlled (i.e. a mechanism to turn CARs “on” or “off”). Switch mechanisms include but are not limited to CAR expression systems that require co-expression of more than one construct to be activated, suicide switches, safety switches, and CARs that require multimerization for activation. In some embodiments, a switch is inducible.

A “Kozak consensus sequence” or “Kozak sequence” is an mRNA sequence that is recognized by a ribosome as a translation start site. A Kozak sequence comprises a start codon (also known as an initiation codon) for initiation of translation and additional flanking nucleotides. The start codon specifies a methionine amino acid at the N-terminus of a translated polypeptide. The Kozak consensus sequence for vertebrates is known in the art (e.g. Kozak, M. 1987 Nucleic Acids Res. 15(20): 8125-48). In some embodiments, Kozak sequences can be modified to be “strong”, meaning that the nucleotide sequence closely matches the consensus sequence, particularly at nucleotides +4 and −3 relative to the number one nucleotide. An “adequate” Kozak sequence has just one of these matching nucleotides while a “weak” Kozak sequence has neither matching nucleotide. The strength of a Kozak sequence directly correlates with the amount of polypeptides translated from an expressed mRNA. In general, strong Kozak sequences result in greater efficiency of translation of an expressed mRNA while fewer polypeptides are transcribed from mRNAs with weak Kozak sequences.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “specific binding” means the contact between an antibody and an antigen with a binding affinity of at least 10⁻⁶ M. In certain aspects, antibodies bind with affinities of at least about 10⁻⁷M, and preferably 10⁻⁸M, 10⁻⁹M, 10⁻¹⁰M, 10⁻¹¹M, or 10⁻¹² M.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell. As used herein, “endogenous” refers to a gene or gene product that is naturally present within a cell and was not introduced through genetic engineering such as transfection or transduction of genetic material.

As used herein, the term “linker sequence” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 flexible polypeptide linker comprising three copies of gly-gly-gly-gly-ser, or equivalents thereof. Non-limiting examples of linker sequences are known in the art, e.g., GGGGSGGGGSGGGG (and equivalents thereof); the tripeptide EFM; or Glu-Phe-Gly-Ala-Gly-Leu-Val-Leu-Gly-Gly-Gln-Phe-Met, and equivalents of each thereof.

As used herein, the term “matrix” refers to a composition such as a gel electrophoresis gel or a matrix commonly used for western blotting (such as membranes made of nitrocellulose or polyvinylidene difluoride), which compositions are useful for electrophoretic and/or immunoblotting techniques, such as Western blotting. As used herein, “solid support” refers to a material, composite material, surface, or functionalized surface capable of supporting a peptide and/or peptide synthesis. Exemplary solid supports include but are not limited to stable beaded gel resins, end group acryloylated long-chain polyethylene glycols, polystyrene resins, and amide bond free PEG-based resins. As used herein, the term “solution” refers to a liquid phase mixture or composition.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

As used herein, the term “WPRE” or “Woodchuck Hepatitis Virus (WHP) Post-transcriptional Regulatory Element” refers to a specific nucleotide fragment associated with this name and any other molecules that have analogous biological function that share at least 70%, or alternatively at least 80% amino acid sequence identity, preferably 90% sequence identity, more preferably at least 95% sequence identity with the WPRE sequence as shown herein. For example, WPRE refers to a region similar to the human hepatitis B virus posttranscriptional regulatory element (HBVPRE) present in the Woodchuck hepatitis virus genomic sequence (GenBank Accession No. J04514), and that the 592 nucleotides from position 1093 to 1684 of this genomic sequence correspond to the post-transcriptional regulatory region (Journal of Virology, Vol. 72, p. 5085-5092, 1998). The analysis using retroviral vectors revealed that WPRE inserted into the 3′-terminal untranslated region of a gene of interest increases the amount of protein produced by 5 to 8 folds. It has also been reported that the introduction of WPRE suppresses mRNA degradation (Journal of Virology, Vol. 73, p. 2886-2892, 1999). In a broad sense, elements such as WPRE that increase the efficiency of amino acid translation by stabilizing mRNAs are also thought to be enhancers.

### List of Abbreviations

CAR: chimeric antigen receptor

HLA: histocompatibility lymphocyte antigen

Ip: intraperitoneal

IRES: internal ribosomal entry site

LHR: leuteinizing hormone receptor

MFI: mean fluorescence intensity

MOI: multiplicity of infection

PBMC: peripheral blood mononuclear cells

PBS: phosphate buffered saline

scFv: single chain variable fragment

WPRE: woodchuck hepatitis virus post-transcriptional regulatory element

The sequences associated with each of the above listed GenBank Accession Nos., UniProt Reference Nos., and references are herein incorporated by reference.

### MODES FOR CARRYING OUT THE DISCLOSURE

Due to the unprecedented results being recently obtained in B-cell lymphomas and leukemia's using autologous treatment with genetically engineered chimeric antigen receptor (CAR) T-cells (Maude, S. L. et al. (2014) New Engl. J. Med. 371:1507-1517; Porter, D. L. et al. (2011) New Engl. J. Med.

CAR T-cells are genetically engineered autologous T-cells in which single chain antibody fragments (scFv) or ligands are attached to the T-cell signaling domain capable of facilitating T-cell activation (Maher, J. (2012) ISRN Oncol. 2012:278093; Curran, K. J. et al. (2012) J. Gene Med. 14:405-415; Fedorov, V. D. et al. (2014) Cancer J. 20:160-165; Barrett, D. M. et al. (2014) Annu. Rev. Med. 65:333-347). CARs combine HLA-independent targeting specificity of a monoclonal antibody with the cytolytic activity and homing properties of activated T-cells. These properties enable the recognition of target cells with reduced HLA expression or down-regulated antigen processing pathways, two common methods tumors employ to evade the host immune response (Jakobsen, M. K. et al. (1995) J. Immunother. Emphasis Tumor Immunol. 17:222-228; Lou, Y. et al. (2008) Clin. Cancer Res. 14:1494-1501; Singh, R. et al. (2007) Cancer Res. 67:1887-1892). CAR-modified T-cells have shown great promise in preclinical and clinical settings as novel therapeutics in various diseases including ovarian carcinomas (Chu, C. S. et al. (2008) Expert Rev. Anticancer Ther. 8:243-257; Chekmasova, A. A. et al. (2010) Discov. Med. 9:62-70; Porter, D. L. et al. (2011) NEJM 365:725-733). To date, CAR T-cells generated against mesothelin (Kelly, R. J. et al. (2012) Mol. Cancer Ther. 11:517-525; Beatty, G. L. et al. (2014) Cancer Immunol. Res. 2:112-120) are currently in clinical trial at the National Cancer Institute (protocol ID: 120111; NCT01583686), the University of Pennsylvania (just enrolling patients), and in China (4 patients completed). These studies are very preliminary and except for the α-folate receptor (Kandalaft, L. E. et al. (2012) J. Transl. Med. 10:157-167) and MUC16 (Chekmasova, A. A. et al. (2010) Clin. Cancer Res. 16:3594-606; Rao, T. D. et al. (2010) Appl. Immunohistochem. Mol. Morphology 18:462-472), no other targets to our knowledge are currently under development for the treatment of ovarian cancer.

To date, CAR modified T-cells to human solid tumors have been constructed against the α-folate receptor, mesothelin, and MUC-CD, PSMA, and other targets but most have some off-target expression of antigen in normal tissues. These constructs have not shown the same exceptional results in patients emphasizing the need for additional studies to identify new targets and methods of CAR T-cell construction that can be used against solid tumors.

Additionally, this disclosure provides antibodies specific to LHR, B7-H4, HLA-G, HLA-DR and methods and compositions relating to the use and production thereof. In addition, this disclosure provides as a chimeric antigen receptor (CAR) comprising an antigen binding domain specific to LHR, B7-H4, HLA-G, HLA-DR, that in some aspects, is the antigen binding domain of an anti-LHR, B7-H4, HLA-G, HLA-DR antibody and methods and compositions relating to the use and production thereof.

Consistent with these principles and discoveries, this disclosure provides the following embodiments.

Provided herein is a chimeric antigen receptor (CAR) comprising, consisting of, or consisting essentially of: (a) an antigen binding domain of an anti-luteinizing hormone receptor (“LHR”) antibody, (b) a CD8 α hinge domain; (c) a CD8 α transmembrane domain; (d) two or more costimulatory signaling regions; and (e) a CD3 zeta signaling domain. In some embodiments, the two or more costimulatory signaling regions are selected from CD27, CD28, 4-IBB (CD 137), OX40, CD30, CD40, PD-1, ICOS, lymphocyte function-associated antigen-1 (LFA-1), CD2, CD7, CD27, LIGHT, NKG2C, and B7-H3.

In some embodiments, the antigen binding domain of the anti-LHR antibody of the CAR comprises, consists of, or consists essentially of an anti-LHR heavy chain (HC) variable region and an anti-LHR light chain (LC) variable region. In some embodiments, the CAR further comprises, consists of, or consists essentially of a linker polypeptide located between the anti-LHR HC variable region and the anti-LHR LC variable region.

In some embodiments, the HC of the anti-LHR antibody of the CAR comprises, consists of, or consists essentially of a CDR1 comprising, consisting of, or consisting essentially of the amino acid sequence of GYSITSGYG or an equivalent of each thereof; and/or a CDR2 comprising, consisting of, or consisting essentially of the amino acid sequence of IHYSGST or an equivalent of each thereof; and/or a CDR3 comprising, consisting of, or consisting essentially of the amino acid sequence of ARSLRY or an equivalent of each thereof. In some embodiments, the LC comprises, consists of, or consists essentially of a CDR1 comprising, consisting of, or consisting essentially of the amino acid sequence of SSVNY or an equivalent of each thereof; and/or a CDR2 comprising, consisting of, or consisting essentially of the amino acid sequence of DTS or an equivalent of each thereof and/or a CDR3 comprising, consisting of, or consisting essentially of the amino acid sequence of HQWSSYPYT or an equivalent of each thereof.

In some embodiments, the HC of the anti-LHR antibody of the CAR comprises, consists of, or consists essentially of a CDR1 comprising, consisting of, or consisting essentially of the amino acid sequence of GFSLTTYG or an equivalent of each thereof and/or a CDR2 comprising the amino acid sequence of IWGDGST or an equivalent of each thereof and/or a CDR3 comprising, consisting of, or consisting essentially of the amino acid sequence of AEGSSLFAY or an equivalent of each thereof. In some embodiments, the LC comprises, consists of, or consists essentially of a CDR1 comprising, consisting of, or consisting essentially of the amino acid sequence of QSLLNSGNQKNY or an equivalent of each thereof and/or a CDR2 comprising, consisting of, or consisting essentially of the amino acid sequence of WAS or an equivalent of each thereof; and/or a CDR3 comprising, consisting of, or consisting essentially of the amino acid sequence of QNDYSYPLT or an equivalent of each thereof.

In some embodiments, the HC of the anti-LHR antibody of the CAR comprises, consists of, or consists essentially of a CDR1 comprising, consisting of, or consisting essentially of the amino acid sequence of GYSFTGYY or an equivalent of each thereof and/or a CDR2 comprising, consisting of, or consisting essentially of the amino acid sequence of IYPYNGVS or an equivalent of each thereof; and/or a CDR3 comprising, consisting of, or consisting essentially of the amino acid sequence of ARERGLYQLRAMDY or an equivalent of each thereof. In some embodiments, the LC comprises, consists of, or consists essentially of a CDR1 comprising, consisting of, or consisting essentially of the amino acid sequence of QSISNN or an equivalent of each thereof; and/or a CDR2 comprising, consisting of, or consisting essentially of the amino acid sequence of NAS or an equivalent of each thereof; and/or a CDR3 comprising, consisting of, or consisting essentially of the amino acid sequence of QQSNSWPYT or an equivalent of each thereof.

In some embodiments, the HC variable region of the anti-LHR antibody of the CAR comprises, consists of, or consists essentially of a polypeptide selected from a sequence disclosed herein or an equivalent of each thereof. In some embodiments, the anti-LHR light chain variable region comprises, consists of, or consists essentially of a polypeptide selected from a sequence disclosed herein or an equivalent of each thereof. In some embodiments, the anti-LHR heavy chain variable region comprises, consists of, or consists essentially of a polypeptide with a consensus sequence selected from a sequence disclosed herein or an equivalent of each thereof. In some embodiments, the anti-LHR light chain variable region comprises, consists of, or consists essentially of a polypeptide with a consensus sequence selected from a sequence disclosed herein or an equivalent of each thereof.

In some embodiments, an equivalent of a polypeptide comprises, consists of, or consists essentially of an polypeptide having at least 80% amino acid identity to the polypeptide or a polypeptide that is encoded by a polynucleotide that hybridizes under conditions of high stringency to the complement of a polynucleotide encoding the polypeptide.

In some embodiments, the CAR further comprises, consists of, or consists essentially of a detectable marker or a purification marker.

In some embodiments, the further comprises, consists of, or consists essentially of an antigen binding domain derived from an antibody against MUC-16 or an antibody against mesothelin.

Also provided herein is an isolated nucleic acid sequence encoding a chimeric antigen receptor (CAR) comprising, consisting of, or consisting essentially of: (a) an antigen binding domain of an anti-luteinizing hormone receptor (“LHR”) antibody, (b) a CD8 α hinge domain; (c) a CD8 α transmembrane domain; (d) two or more costimulatory signaling regions; and (e) a CD3 zeta signaling domain. In some embodiments, the isolated nucleic acid sequence comprises, consists of, or consists essentially of a sequence selected from any one of a sequence disclosed herein, or an equivalent of each thereof.

In some embodiments, the isolated nucleic acid sequence further comprises, consists of, or consists essentially of a Kozak consensus sequence located upstream of the antigen binding domain of the anti-LHR antibody or an enhancer.

In some embodiments, the isolated nucleic acid sequence further comprises, consists of, or consists essentially of an antibiotic resistance polynucleotide.

In some embodiments, the isolated nucleic acid sequence further comprises, consists of, or consists essentially of a switch mechanism for controlling expression and/or activation of the CAR.

Also provided herein is a vector comprising, consisting of, or consisting essentially of an isolated nucleic acid sequence encoding a chimeric antigen receptor (CAR) comprising, consisting of, or consisting essentially of: (a) an antigen binding domain of an anti-luteinizing hormone receptor (“LHR”) antibody, (b) a CD8 α hinge domain; (c) a CD8 α transmembrane domain; (d) two or more costimulatory signaling regions; and (e) a CD3 zeta signaling domain. In some embodiments, the vector is a plasmid. In some embodiments, the vector is selected from a group of a retroviral vector, a lentiviral vector, an adenoviral vector, and an adeno-associated viral vector. In some embodiments, the vector is a CRISPR vector or a vector comprising CRISPR.

Also provided herein is an isolated cell comprising a chimeric antigen receptor (CAR) comprising, consisting of, or consisting essentially of: (a) an antigen binding domain of an anti-luteinizing hormone receptor (“LHR”) antibody, (b) a CD8 α hinge domain; (c) a CD8 α transmembrane domain; (d) two or more costimulatory signaling regions; and (e) a CD3 zeta signaling domain. In some embodiments, provided herein is an isolated cell comprising, consisting of, or consisting essentially of an; an isolated nucleic acid sequence encoding a chimeric antigen receptor (CAR) comprising, consisting of, or consisting essentially of: (a) an antigen binding domain of an anti-luteinizing hormone receptor (“LHR”) antibody, (b) a CD8 α hinge domain; (c) a CD8 α transmembrane domain; (d) two or more costimulatory signaling regions; and (e) a CD3 zeta signaling domain. In some embodiments, provided herein is an isolated cell comprising, consisting of, or consisting essentially of a vector comprising, consisting of, or consisting essentially of an isolated nucleic acid sequence encoding a chimeric antigen receptor (CAR) comprising, consisting of, or consisting essentially of: (a) an antigen binding domain of an anti-luteinizing hormone receptor (“LHR”) antibody, (b) a CD8 α hinge domain; (c) a CD8 α transmembrane domain; (d) two or more costimulatory signaling regions; and (e) a CD3 zeta signaling domain. In some embodiments, the isolated cell comprises, consists of, or consists essentially of one or more of the CAR, isolated nucleic acid, or vector.

In some embodiments, the isolated cell is an immune cell. In some embodiments, the immune cell is a T-cell or a natural killer (NK) cell. In some embodiments, the isolated cell is a T-cell precursor or an NK cell precursor.

Provided herein is a composition comprising, consisting of, or consisting essentially of a carrier and one or more of: a CAR comprising, consisting of, or consisting essentially of: (a) an antigen binding domain of an anti-luteinizing hormone receptor (“LHR”) antibody, (b) a CD8 α hinge domain; (c) a CD8 α transmembrane domain; (d) two or more costimulatory signaling regions; and (e) a CD3 zeta signaling domain, an isolated nucleic acid encoding the CAR, a vector comprising, consisting of, or consisting essentially of the isolated nucleic acid, or an isolated cell comprising the CAR, isolated nucleic acid, and/or vector.

In some embodiments, the composition further comprises, consists of, or consists essentially of an antigen binding fragment capable of binding a peptide, wherein the peptide comprises an LHR protein or a fragment thereof. In some embodiments, the peptide is associated with a cell. In some embodiments, the peptide is bound to a solid support. In some embodiments, the peptide is disposed in a solution. In some embodiments, the peptide is associated with a matrix.

Also provided herein is a method of producing anti-LHR CAR expressing cells comprising, consisting of, or consisting essentially of: (i) introducing a population of immune cells with a nucleic acid sequence encoding a CAR comprising, consisting of, or consisting essentially of: (a) an antigen binding domain of an anti-luteinizing hormone receptor (“LHR”) antibody, (b) a CD8 α hinge domain; (c) a CD8 α transmembrane domain; (d) two or more costimulatory signaling regions; and (e) a CD3 zeta signaling domain; and (ii) selecting a subpopulation of immune cells that have been successfully transduced with said nucleic acid sequence of step (i) thereby producing anti-LHR CAR expressing cells.

In some embodiments of the method, the immune cells are T-cells. In some embodiments of the method, the population of T-cells have been modified to reduce or eliminate expression of endogenous T-cell receptors.

In some embodiments of the method, the population of T-cells were modified using a method that employs RNA interference or CRISPR.

Also provided herein is a method of inhibiting the growth of a tumor and/or treating a cancer in a subject in need thereof, comprising, consisting of, or consisting essentially of administering to the subject an effective amount of isolated anti-LHR CAR expressing cells according to any of the embodiments provided herein.

In some embodiments of the method, the anti-LHR CAR expressing cells are autologous or allogenic to the subject being treated.

In some embodiments of the method, the tumor or cancer expresses or overexpresses LHR. In some embodiments, the tumor is a solid tumor, optionally an ovarian tumor or a prostate cancer tumor and/or the cancer is and ovarian cancer or a prostate cancer.

In some embodiments of the method, the subject in need thereof is a human, an animal, a non-human primate, a dog, cat, a sheep, a mouse, a horse, or a cow.

Provided herein is a chimeric antigen receptor (CAR) comprising, consisting of, or consisting essentially of: (a) an antigen binding domain of an anti-B7-H4 antibody, (b) a CD8 α hinge domain; (c) a CD8 α transmembrane domain; (d) two or more costimulatory signaling regions; and (e) a CD3 zeta signaling domain. In some embodiments, the two or more costimulatory signaling regions are selected from CD27, CD28, 4-IBB (CD 137), OX40, CD30, CD40, PD-1, ICOS, lymphocyte function-associated antigen-1 (LFA-1), CD2, CD7, CD27, LIGHT, NKG2C, and B7-H3.

In some embodiments, the antigen binding domain of the anti-B7-H4 antibody of the CAR comprises, consists of, or consists essentially of an anti-B7-H4 heavy chain (HC) variable region and an anti-B7-H4 light chain (LC) variable region. In some embodiments, the CAR further comprises a linker polypeptide located between the anti-B7-H4 HC variable region and the anti-B7-H4 LC variable region.

In some embodiments, the HC of the anti-B7-H4 antibody comprises, consists of, or consists essentially of: a CDR1 comprising, consisting of, or consisting essentially of the amino acid sequence of GXTF or an equivalent of each thereof; and/or a CDR2 comprising, consisting of, or consisting essentially of the amino acid sequence of (i) ISSXXXT, (ii) INPNNGGT, or an equivalent of each thereof; and/or a CDR3 comprising, consisting of, or consisting essentially of the amino acid sequence of ARPXYY or an equivalent of each thereof; and/or the LC comprises, consists of, or consists essentially of a CDR1 comprising, consisting of, or consisting essentially of the amino acid sequence of (i) QSIVHXNGTY, (ii) ENIGSY, or an equivalent of each thereof; and/or a CDR2 comprising, consisting of, or consisting essentially of the amino acid sequence of (i) KVS, (ii) AAT, or an equivalent of each thereof; and/or a CDR3 comprising, consisting of, or consisting essentially of the amino acid sequence of (i) FQGSXVPXT, (ii) QHYYSTLVT, or an equivalent of each thereof.

In some embodiments, the HC of the anti-B7-H4 antibody comprises, consists of, or consists essentially of, consists of, or consists essentially of: a CDR1 comprising, consisting of, or consisting essentially of the amino acid sequence of (i) GFTFSSFG, (ii) GFTFSSYG, (iii) GYTFTDY or an equivalent of each thereof; and/or a CDR2 comprising, consisting of, or consisting essentially of the amino acid sequence of (i) ISSGSSTL, (ii) ISSSNSTI, or an equivalent of each thereof; and/or a CDR3 comprising, consisting of, or consisting essentially of the amino acid sequence of (i) ARPLYYYGSVMDY, (ii) ARPYYYGSSYDY, or an equivalent of each thereof and/or the LC comprises, consists of, or consists essentially of a CDR1 comprising, consisting of, or consisting essentially of the amino acid sequence of (i) QSIVHRNGNTY, (ii) QSIVHSNGNTY,or an equivalent of each thereof; and/or a CDR3 comprising, consisting of, or consisting essentially of the amino acid sequence of (i) FQGSYVPPT, (ii) FQGSHVPLT, or an equivalent of each thereof.

In some embodiments, the anti-B7-H4 heavy chain variable region of the CAR comprises, consists of, or consists essentially of a polypeptide selected from a sequence disclosed herein or an equivalent of each thereof. In some embodiments, the anti-B7-H4 light chain variable region of the CAR comprises, consists of, or consists essentially of a polypeptide selected from a sequence disclosed herein or an equivalent of each thereof. In some embodiments, the anti-B7-H4 heavy chain variable region of the CAR comprises, consists of, or consists essentially of a polypeptide with a consensus sequence selected from a sequence disclosed herein or an equivalent of each thereof. In some embodiments, the anti-B7-H4 light chain variable region comprises, consists of, or consists essentially of a polypeptide with a consensus sequence selected from a sequence disclosed herein or an equivalent of each thereof.

In some embodiments, the CAR further comprises, consists of, or consists essentially of a detectable marker or a purification marker.

Also provided herein is an isolated nucleic acid sequence encoding a chimeric antigen receptor (CAR) comprising, consisting of, or consisting essentially of: (a) an antigen binding domain of an anti-B7-H4 antibody, (b) a CD8 α hinge domain; (c) a CD8 α transmembrane domain; (d) two or more costimulatory signaling regions; and (e) a CD3 zeta signaling domain. In some embodiments, the isolated nucleic acid sequence comprises a sequence selected from any one of a sequence disclosed herein, or an equivalent of each thereof.

In some embodiments, the isolated nucleic acid sequence further comprises, consists of, or consists essentially of a Kozak consensus sequence located upstream of the antigen binding domain of the anti-B7-H4 antibody or an enhancer.

In some embodiments, the isolated nucleic acid sequence further comprises, consists of, or consists essentially of an antibiotic resistance polynucleotide.

In some embodiments, the isolated nucleic acid sequence further comprises, consists of, or consists essentially of a switch mechanism for controlling expression and/or activation of the CAR.

Provided herein is a vector comprising, consisting of, or consisting essentially of an isolated nucleic acid sequence encoding a chimeric antigen receptor (CAR) comprising, consisting of, or consisting essentially of: (a) an antigen binding domain of an anti-B7-H4 antibody, (b) a CD8 α hinge domain; (c) a CD8 α transmembrane domain; (d) two or more costimulatory signaling regions; and (e) a CD3 zeta signaling domain. In some embodiments, the vector is a plasmid. In some embodiments, the vector is selected from a retroviral vector, a lentiviral vector, an adenoviral vector, and an adeno-associated viral vector. In some embodiments, the vector is a CRISPR vector.

Also provided herein is an isolated cell comprising, consisting of, or consisting essentially of, a chimeric antigen receptor (CAR) comprising, consisting of, or consisting essentially of: (a) an antigen binding domain of an anti-B7-H4 antibody, (b) a CD8 α hinge domain; (c) a CD8 α transmembrane domain; (d) two or more costimulatory signaling regions; and (e) a CD3 zeta signaling domain; and/or an isolated nucleic acid encoding the CAR; and/or a vector comprising, consisting of, or consisting essentially of the isolated nucleic acid. In some embodiments, the isolated cell is an immune cell. In some embodiments, the immune cell is a T-cell or a natural killer (NK) cell. In some embodiments, the isolated cell is a T-cell precursor or an NK cell precursor.

Provided herein is a composition comprising, consisting of, or consisting essentially of a carrier and one or more of: a chimeric antigen receptor (CAR) comprising, consisting of, or consisting essentially of: (a) an antigen binding domain of an anti-B7-H4 antibody, (b) a CD8 α hinge domain; (c) a CD8 α transmembrane domain; (d) two or more costimulatory signaling regions; and (e) a CD3 zeta signaling domain; an isolated nucleic acid sequence encoding the CAR; a vector comprising the isolated nucleic acid sequence; and/or an isolated cell comprising the CAR, vector, or isolated nucleic acid sequence.

In some embodiments, the composition further comprises an antigen binding fragment capable of binding a peptide, wherein the peptide comprises an B7-H4 protein or a fragment thereof. In some embodiments, the peptide is associated with a cell. In some embodiments, the peptide is bound to a solid support. In some embodiments, the peptide is disposed in a solution. In some embodiments, the peptide is associated with a matrix.

Provided herein is a method of producing anti-B7-H4 CAR expressing cells comprising: (i) introducing a population of immune cells with a nucleic acid sequence encoding a chimeric antigen receptor (CAR) comprising, consisting of, or consisting essentially of: (a) an antigen binding domain of an anti-B7-H4 antibody, (b) a CD8 α hinge domain; (c) a CD8 α transmembrane domain; (d) two or more costimulatory signaling regions; and (e) a CD3 zeta signaling domain; and (ii) selecting a subpopulation of immune cells that have been successfully transduced with said nucleic acid sequence of step (i) thereby producing anti-B7-H4 CAR expressing cells. In some embodiments, the immune cells are T-cells. In some embodiments, the population of T-cells have been modified to reduce or eliminate expression of endogenous T-cell receptors. In some embodiments, the population of T-cells were modified using a method that employs RNA interference or CRISPR.

Also provided is a method of inhibiting the growth of a tumor and/or treating a cancer in a subject in need thereof, comprising administering to the subject an effective amount of anti-B7-H4 CAR expressing cells comprising, consisting of, or consisting essentially of, a chimeric antigen receptor (CAR) comprising, consisting of, or consisting essentially of: (a) an antigen binding domain of an anti-B7-H4 antibody, (b) a CD8 α hinge domain; (c) a CD8 α transmembrane domain; (d) two or more costimulatory signaling regions; and (e) a CD3 zeta signaling domain; and/or an isolated nucleic acid encoding the CAR; and/or a vector comprising, consisting of, or consisting essentially of the isolated nucleic acid. In some embodiments, the anti-B7-H4 CAR expressing cells are autologous or allogenic to the subject being treated. In some embodiments, the tumor or cancer expresses or overexpresses B7-H4. In some embodiments, the tumor is a solid tumor, optionally, a breast, colon, or chorio-carcinoma tumor and/or the cancer is a breast, color or a chorio-carcinoma. In some embodiments, the subject is a human, an animal, a non-human primate, a dog, cat, a sheep, a mouse, a horse, or a cow.

Provided herein is a chimeric antigen receptor (CAR) comprising, consisting of, or consisting essentially of: (a) an antigen binding domain of an anti-HLA-G antibody, (b) a CD8 α hinge domain; (c) a CD8 α transmembrane domain; (d) two or more costimulatory signaling regions; and (e) a CD3 zeta signaling domain. In some embodiments, the two or more costimulatory signaling regions are selected from CD27, CD28, 4-IBB (CD 137), OX40, CD30, CD40, PD-1, ICOS, lymphocyte function-associated antigen-1 (LFA-1), CD2, CD7, CD27, LIGHT, NKG2C, and B7-H3.

In some embodiments, the antigen binding domain of the anti-HLA-G antibody comprises, consists of, or consists essentially of an anti-HLA-G heavy chain (HC) variable region and an anti-HLA-G light chain (LC) variable region. In some embodiments, the CAR further comprises, consists of, or consists essentially of a linker polypeptide located between the anti-HLA-G HC variable region and the anti-HLA-G LC variable region.

In some embodiments, the HC of the anti-HLA-G antibody comprises, consists of, or consists essentially of: a CDR1 comprising, consisting of, or consisting essentially of the amino acid sequence of (i) GFNIKDTY, (ii) GFTFNTYA, or an equivalent of each thereof; and/or a CDR2 comprising, consisting of, or consisting essentially of the amino acid sequence of (i) IDPANGNT, (ii) IRSKSNNYAT, or an equivalent of each thereof; and/or a CDR3 comprising, consisting of, or consisting essentially of the amino acid sequence of (i) ARSYYGGFAY, (ii) VRGGYWSFDV, or an equivalent of each thereof; and/or the LC comprises, consists of, or consists essentially of a CDR1 comprising, consisting of, or consisting essentially of the amino acid sequence of (i) KSVSTSGYSY, (ii) KSLLHSNGNTY, or an equivalent of each thereof; and/or a CDR2 comprising, consisting of, or consisting essentially of the amino acid sequence of (i) LVS, (ii) RMS, or an equivalent of each thereof and/or a CDR3 comprising, consisting of, or consisting essentially of the amino acid sequence of (i) QHSRELPRT, (ii) MQHLEYPYT, or an equivalent of each thereof.

In some embodiments, the anti-HLA-G heavy chain variable region of the CAR comprises, consists of, or consists essentially of a polypeptide selected from a sequence disclosed herein or an equivalent of each thereof. In some embodiments, the anti-HLA-G light chain variable region comprises, consists of, or consists essentially of a polypeptide selected from a sequence disclosed herein or an equivalent of each thereof. In some embodiments, the anti-HLA-G heavy chain variable region comprises, consists of, or consists essentially of a polypeptide with a consensus sequence selected from a sequence disclosed herein or an equivalent of each thereof. In some embodiments, the anti-HLA-G light chain variable region comprises, consists of, or consists essentially of a polypeptide with a consensus sequence selected from a sequence disclosed herein or an equivalent of each thereof.

In some embodiments, the CAR further comprises a detectable marker or a purification marker.

Provided herein is an isolated nucleic acid sequence encoding a chimeric antigen receptor (CAR) comprising, consisting of, or consisting essentially of: (a) an antigen binding domain of an anti-HLA-G antibody, (b) a CD8 α hinge domain; (c) a CD8 α transmembrane domain; (d) two or more costimulatory signaling regions; and (e) a CD3 zeta signaling domain. In some embodiments, the nucleic acid sequence comprises a sequence selected from any one of a sequence disclosed herein, or an equivalent of each thereof.

In some embodiments, the isolated nucleic acid further comprises, consists of, or consists essentially of a Kozak consensus sequence located upstream of the antigen binding domain of the anti-HLA-G antibody or an enhancer. In some embodiments, the isolated nucleic acid further comprises, consists of, or consists essentially of an antibiotic resistance polynucleotide. In some embodiments, the isolated nucleic acid further comprises, consists of, or consists essentially of a switch mechanism for controlling expression and/or activation of the CAR.

Also provided herein is a vector comprising, consisting of, or consisting essentially of an isolated nucleic acid sequence encoding a chimeric antigen receptor (CAR) comprising, consisting of, or consisting essentially of: (a) an antigen binding domain of an anti-HLA-G antibody, (b) a CD8 α hinge domain; (c) a CD8 α transmembrane domain; (d) two or more costimulatory signaling regions; and (e) a CD3 zeta signaling domain. In some embodiments, the vector is a plasmid. In some embodiments, the vector is selected from a group consisting of a retroviral vector, a lentiviral vector, an adenoviral vector, and an adeno-associated viral vector. In some embodiments, the vector is a CRISPR vector or a vector comprising CRISPR.

Provided herein is an isolated cell comprising, consisting of, or consisting essentially of a chimeric antigen receptor (CAR) comprising, consisting of, or consisting essentially of: (a) an antigen binding domain of an anti-HLA-G antibody, (b) a CD8 α hinge domain; (c) a CD8 α transmembrane domain; (d) two or more costimulatory signaling regions; and (e) a CD3 zeta signaling domain; and/or an isolated nucleic acid sequence encoding the CAR; and/or a vector comprising, consisting of, or consisting essentially of the isolated nucleic acid sequence. In some embodiments, the isolated cell is an immune cell. In some embodiments, the isolated cell is a T-cell or a natural killer (NK) cell. In some embodiments, the isolated cell is a T-cell precursor or an NK cell precursor.

Also provided herein is a composition comprising, consisting of, or consisting essentially of a carrier and one or more of: a chimeric antigen receptor (CAR) comprising, consisting of, or consisting essentially of: (a) an antigen binding domain of an anti-HLA-G antibody, (b) a CD8 α hinge domain; (c) a CD8 α transmembrane domain; (d) two or more costimulatory signaling regions; and (e) a CD3 zeta signaling domain; and/or an isolated nucleic acid sequence encoding the CAR; and/or a vector comprising, consisting of, or consisting essentially of the isolated nucleic acid sequence; and/or an isolated cell comprising, consisting of, or consisting essentially of the CAR, isolated nucleic acid sequence, or vector.

In some embodiments, the composition further comprises, consists of, or consists essentially of an antigen binding fragment capable of binding a peptide, wherein the peptide comprises an HLA-G protein or a fragment thereof. In some embodiments, the peptide is associated with a cell. In some embodiments, the peptide is bound to a solid support. In some embodiments, the peptide is disposed in a solution. In some embodiments, the peptide is associated with a matrix.

Provided herein is a method of producing anti-HLA-G CAR expressing cells comprising, consisting of, or consisting essentially of: (i) introducing a population of immune cells with a nucleic acid sequence encoding a chimeric antigen receptor (CAR) comprising, consisting of, or consisting essentially of: (a) an antigen binding domain of an anti-HLA-G antibody, (b) a CD8 α hinge domain; (c) a CD8 α transmembrane domain; (d) two or more costimulatory signaling regions; and (e) a CD3 zeta signaling domain; and (ii) selecting a subpopulation of immune cells that have been successfully transduced with said nucleic acid sequence of step (i) thereby producing anti-HLA-G CAR expressing cells. In some embodiments, the immune cells are T-cells. In some embodiments, the population of T-cells have been modified to reduce or eliminate expression of endogenous T-cell receptors. In some embodiments, the population of T-cells were modified using a method that employs RNA interference or CRISPR.

Also provided herein is a method of inhibiting the growth of a tumor and/or treating a cancer in a subject in need thereof, comprising, consisting of, or consisting essentially of administering to the subject an effective amount of anti-HLA-G CAR expressing cells comprising, consisting of, or consisting essentially of a chimeric antigen receptor (CAR) comprising, consisting of, or consisting essentially of: (a) an antigen binding domain of an anti-HLA-G antibody, (b) a CD8 α hinge domain; (c) a CD8 α transmembrane domain; (d) two or more costimulatory signaling regions; and (e) a CD3 zeta signaling domain; and/or an isolated nucleic acid sequence encoding the CAR; and/or a vector comprising, consisting of, or consisting essentially of the isolated nucleic acid sequence. In some embodiments, the anti-HLA-G CAR expressing cells are autologous or allogenic to the subject being treated.

In some embodiments of the method, the tumor or cancer expresses or overexpresses HLA-G. In some embodiments, the tumor is a solid tumor, optionally a thyroid tumor, an ovarian tumor or a prostate cancer tumor and/or the cancer is a thyroid cancer, ovarian cancer, or prostate cancer.

In some embodiments of the method, the subject is a human, an animal, a non-human primate, a dog, cat, a sheep, a mouse, a horse, or a cow.

Provided herein is a chimeric antigen receptor (CAR) comprising, consisting of, or consisting essentially of: (a) an antigen binding domain of an anti-HLA-DR antibody, (b) a CD8 α hinge domain; (c) a CD8 α transmembrane domain; (d) two or more costimulatory signaling regions; and (e) a CD3 zeta signaling domain. In some embodiments, the two or more costimulatory signaling regions are selected from CD27, CD28, 4-IBB (CD 137), OX40, CD30, CD40, PD-1, ICOS, lymphocyte function-associated antigen-1 (LFA-1), CD2, CD7, CD27, LIGHT, NKG2C, and B7-H3.

In some embodiments, the antigen binding domain of the anti-HLA-DR antibody comprises, consists of, or consists essentially of an anti-HLA-DR heavy chain (HC) variable region and an anti-HLA-DR light chain (LC) variable region. In some embodiments, the CAR further comprises, consists of, or consists essentially of a linker polypeptide located between the anti-HLA-DR HC variable region and the anti-HLA-DR LC variable region.

In some embodiments, the HC of the anti-HLA-DR antibody of the CAR comprises, consists of, or consists essentially of: a CDR1 comprising, consisting of, or consisting essentially of the amino acid sequence of (i) a CDRH1 of a Lym-1 antibody, (ii) a CDRH1 of a Lym-2 antibody, or an equivalent of each thereof; and/or a CDR2 comprising, consisting of, or consisting essentially of the amino acid sequence of (i) a CDRH2 of a Lym-1 antibody, (ii) a CDRH2 of a Lym-2 antibody, or an equivalent of each thereof; and/or a CDR3 comprising, consisting of, or consisting essentially of the amino acid sequence of (i(i) a CDRH3 of a Lym-1 antibody, (ii) a CDRH1 of a Lym-2 antibody, or an equivalent of each thereof; and/or the LC comprises, consists of, or consists essentially of a CDR1 comprising, consisting of, or consisting essentially of the amino acid sequence of (i) a CDRL1 of a Lym-1 antibody, (ii) a CDRL1 of a Lym-2 antibody, or an equivalent of each thereof; and/or a CDR2 comprising, consisting of, or consisting essentially of the amino acid sequence of (i) a CDRL2 of a Lym-1 antibody, (ii) a CDRL2 of a Lym-2 antibody, or an equivalent of each thereof; and/or a CDR3 comprising, consisting of, or consisting essentially of the amino acid sequence of (i) a CDRL3 of a Lym-1 antibody, (ii) a CDRL3 of a Lym-2 antibody, or an equivalent of each thereof.

In some embodiments, the anti-HLA-DR heavy chain variable region comprises a polypeptide selected from a sequence disclosed herein or an equivalent of each thereof. In some embodiments, the anti-HLA-DR light chain variable region comprises a polypeptide selected from a sequence disclosed herein or an equivalent of each thereof. In some embodiments, the anti-HLA-DR heavy chain variable region comprises a polypeptide with a consensus sequence selected from a sequence disclosed herein or an equivalent of each thereof. In some embodiments, the anti-HLA-DR light chain variable region comprises a polypeptide with a consensus sequence selected from a sequence disclosed herein or an equivalent of each thereof.

In some embodiments, the CAR further comprises, consists of, or consists essentially of a detectable marker or a purification marker.

Provided herein is an isolated nucleic acid sequence encoding a chimeric antigen receptor (CAR) comprising, consisting of, or consisting essentially of: (a) an antigen binding domain of an anti-HLA-DR antibody, (b) a CD8 α hinge domain; (c) a CD8 α transmembrane domain; (d) two or more costimulatory signaling regions; and (e) a CD3 zeta signaling domain. In some embodiments, the nucleic acid sequence comprises a sequence selected from any one of a sequence disclosed herein, or an equivalent of each thereof.

In some embodiments, the isolated nucleic acid further comprises, consists of, or consists essentially of a Kozak consensus sequence located upstream of the antigen binding domain of the anti-HLA-DR antibody or an enhancer.

In some embodiments, the isolated nucleic acid further comprises, consists of, or consists essentially of an antibiotic resistance polynucleotide.

In some embodiments, the isolated nucleic acid further comprises, consists of, or consists essentially of a switch mechanism for controlling expression and/or activation of the CAR.

Provided herein is a vector comprising, consisting of, or consisting essentially of a isolated isolated nucleic acid sequence encoding a chimeric antigen receptor (CAR) comprising, consisting of, or consisting essentially of: (a) an antigen binding domain of an anti-HLA-DR antibody, (b) a CD8 α hinge domain; (c) a CD8 α transmembrane domain; (d) two or more costimulatory signaling regions; and (e) a CD3 zeta signaling domain. In some embodiments, the vector is a plasmid. In some embodiments, the vector is selected from a group consisting of a retroviral vector, a lentiviral vector, an adenoviral vector, and an adeno-associated viral vector. In some embodiments, the vector is a CRISPR vector or a vector comprising CRISPR.

Provided herein is an isolated cell comprising a chimeric antigen receptor (CAR) comprising, consisting of, or consisting essentially of: (a) an antigen binding domain of an anti-HLA-DR antibody, (b) a CD8 α hinge domain; (c) a CD8 α transmembrane domain; (d) two or more costimulatory signaling regions; and (e) a CD3 zeta signaling domain; and/or an isolated nucleic acid encoding the CAR; and/or a vector of comprising, consisting of, or consisting essentially of the isolated nucleic acid encoding the CAR. In some embodiments, the isolated cell is an immune cell. In some embodiments, the immune cell is a T-cell or a natural killer (NK) cell. In some embodiments, the isolated cell is a T-cell or NK-cell precursor.

Provided herein is a composition comprising a carrier and one or more of: a chimeric antigen receptor (CAR) comprising, consisting of, or consisting essentially of: (a) an antigen binding domain of an anti-HLA-DR antibody, (b) a CD8 α hinge domain; (c) a CD8 α transmembrane domain; (d) two or more costimulatory signaling regions; and (e) a CD3 zeta signaling domain; and/or an isolated nucleic acid encoding the CAR; and/or a vector of comprising, consisting of, or consisting essentially of the isolated nucleic acid sequence encoding the CAR; and/or an isolated cell comprising at least one of the CAR, isolated nucleic acid sequence, and vector.

In some embodiments, the composition further comprises, consists of, or consists essentially of an antigen binding fragment capable of binding a peptide, wherein the peptide comprises an HLA-DR protein or a fragment thereof. In some embodiments, the peptide is associated with a cell. In some embodiments, the peptide is bound to a solid support. In some embodiments, the peptide is disposed in a solution. In some embodiments, the peptide is associated with a matrix.

Provided herein is a method of producing anti-HLA-DR CAR expressing cells comprising, consisting of, or consisting essentially of a chimeric antigen receptor (CAR) comprising, consisting of, or consisting essentially of: (a) an antigen binding domain of an anti-HLA-DR antibody, (b) a CD8 α hinge domain; (c) a CD8 α transmembrane domain; (d) two or more costimulatory signaling regions; and (e) a CD3 zeta signaling domain; and/or an isolated nucleic acid encoding the CAR; and/or a vector of comprising, consisting of, or consisting essentially of the isolated nucleic acid encoding the CAR, the method comprising: (i) introducing a population of immune cells with a nucleic acid sequence encoding the CAR of any one of claims 119 to 129; and (ii) selecting a subpopulation of immune cells that have been successfully transduced with said nucleic acid sequence of step (i) thereby producing anti-HLA-DR CAR expressing cells. In some embodiments, the immune cells are T-cells. In some embodiments, the population of T-cells have been modified to reduce or eliminate expression of endogenous T-cell receptors. In some embodiments, the population of T-cells were modified using a method that employs RNA interference or CRISPR.

Also provided herein is a method of inhibiting the growth of a tumor and/or treating a cancer in a subject in need thereof, comprising administering to the subject an effective amount of the anti-HLA-DR CAR expressing cells comprising, consisting of, or consisting essentially of a chimeric antigen receptor (CAR) comprising, consisting of, or consisting essentially of: (a) an antigen binding domain of an anti-HLA-DR antibody, (b) a CD8 α hinge domain; (c) a CD8 α transmembrane domain; (d) two or more costimulatory signaling regions; and (e) a CD3 zeta signaling domain; and/or an isolated nucleic acid encoding the CAR; and/or a vector of comprising, consisting of, or consisting essentially of the isolated nucleic acid encoding the CAR. In some embodiments, the anti-HLA-DR CAR expressing cells are autologous or allogenic to the subject being treated.

In some embodiments of the method, the tumor or cancer expresses or overexpresses HLA-DR as compared to a normal, non-cancerous counterpart cell. In some embodiments, the tumor is a B-cell lymphoma tumor or a leukemia tumor and/or the cancer is a B-cell lymphoma or a leukemia.

In some embodiments of the method, the subject is a human, an animal, a non-human primate, a dog, cat, a sheep, a mouse, a horse, or a cow.

Provided herein is a kit comprising one or more of a CAR, isolated nucleic acid sequence, vector, isolated cell, and composition disclosed herein and instructions for use according to one or more methods disclosed herein.

### Antibodies and Uses Thereof

I. Compositions

The general structure of antibodies is known in the art and will only be briefly summarized here. An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus once a set of CDR sequences (i.e. the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

In one embodiment, the disclosure provides an isolated antibody comprising a heavy chain (HC) immunoglobulin variable domain sequence and a light chain (LC) immunoglobulin variable domain sequence, wherein the antibody binds to an epitope of a luteinizing hormone receptor (LHR), B7-H4, HLA-G, or HLA-DR.

### Anti-LHR Antibodies

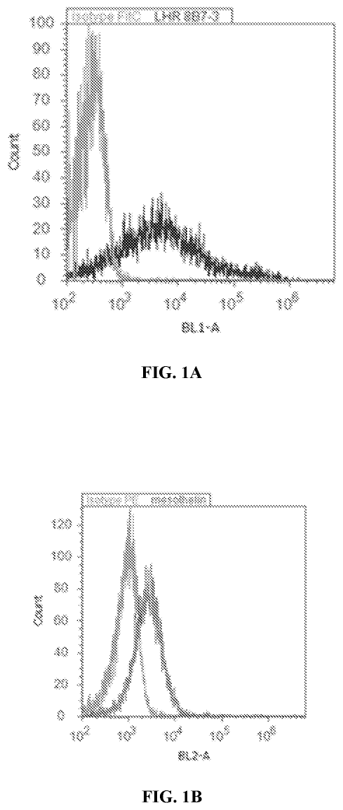

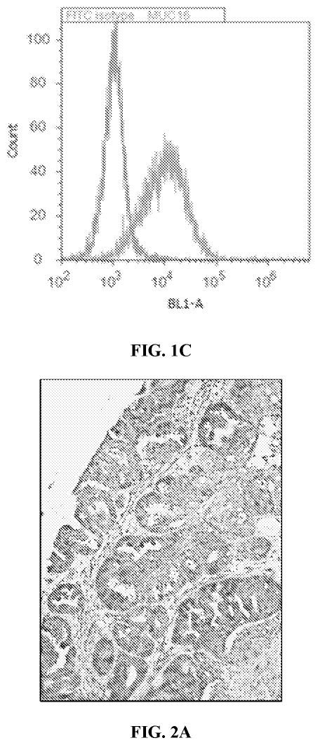

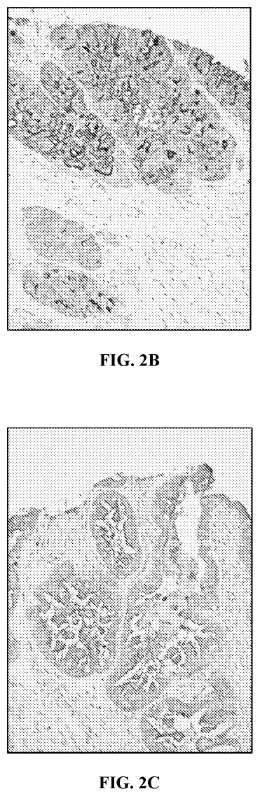

As described in more detail below, the inventors have demonstrated that LHR is a potent target for CAR T-cell therapy. As shown below in Table 1 and FIG. 1, flow cytometric studies utilizing 9 well established human ovarian cell lines showed LHR to be an excellent target compared to mesothelin and MUC16, which were only positive on half or less of the cell lines tested. These targets were also tested on a multi-block slide of human ovarian cancers by immunohistochemistry as shown in Table 2. Consistent with the flow cytometric results, LHR positivity was more consistently seen than mesothelin and MUC16 positivity by these methods regardless of the stage or grade of tumor tested. As shown in FIG. 2, the immunohistochemical staining patterns were somewhat different with each antibody. Both the MUC16 and mesothelin antibodies tended to stain the luminal surfaces of tumor nodules and did not stain the cell surface of all cells especially those more on the periphery of tumor nodules. By contrast, the LHR antibody stained both the cytoplasm and cell surface and tended to stain all the cells of the tumor nodules. Finally, the off-target staining of each antibody was tested on multi-tissue arrays of normal tissues. The results of these studies shown in Table 3 below and show that all three targets have limited reactivity on normal tissues.

In one aspect, the HC of the antibody comprises or alternatively consists essentially of, or yet further consists of one or more of a CDR1 comprising the amino acid sequence of GYSITSGYG or an equivalent of each thereof; and/or a CDR2 comprising the amino acid sequence of IHYSGST or an equivalent of each thereof; and/or a CDR3 comprising the amino acid sequence of ARSLRY or an equivalent of each thereof; and/or the LC comprises the antibody of comprises or alternatively consists essentially of, or yet further consists of a CDR1 comprising the amino acid sequence of SSVNY or an equivalent of each thereof; and/or a CDR2 comprising the amino acid sequence of DTS or an equivalent of each thereof; and/or a CDR3 comprising the amino acid sequence of HQWSSYPYT or an equivalent of each thereof.

In one aspect, the antibody comprises a HC that comprises, or alternatively consists essentially of, or yet further consists of a one or more of: a CDR1 comprising the amino acid sequence of GFSLTTYG or an equivalent of each thereof; and/or a CDR2 comprising the amino acid sequence of IWGDGST or an equivalent of each thereof; and/or a CDR3 comprising the amino acid sequence of AEGSSLFAY or an equivalent of each thereof; and/or the LC of the antibody comprises, or alternatively consists essentially of, or yet further consists of a CDR1 comprising the amino acid sequence of QSLLNSGNQKNY or an equivalent of each thereof; and/or a CDR2 comprising the amino acid sequence of WAS or an equivalent of each thereof; and/or a CDR3 comprising the amino acid sequence of QNDYSYPLT or an equivalent of each thereof.

In another aspect, the HC of the antibody comprises, or alternatively consists essentially of, or yet further consists of one or more of: a CDR1 comprising the amino acid sequence of GYSFTGYY or an equivalent of each thereof; and/or a CDR2 comprising the amino acid sequence of IYPYNGVS or an equivalent of each thereof; and/or a CDR3 comprising the amino acid sequence of ARERGLYQLRAMDY or an equivalent of each thereof and/or the LC of the antibody comprises, or alternatively consists essentially of, or yet further consists of a CDR1 comprising the amino acid sequence of QSISNN or an equivalent of each thereof; and/or a CDR2 comprising the amino acid sequence of NAS or an equivalent of each thereof; and/or a CDR3 comprising the amino acid sequence of QQSNSWPYT or an equivalent of each thereof.

In one aspect, the disclosure provides an isolated anti-LHR antibody which is generated against a LHR fragment.

In one embodiment, the LHR fragment is part of LHR G protein with the following amino acid sequence:

REALCPEPCNCVPDGALRCPGPTAGLTRLSLAYLPVKVIPSQAFRGLNEV
IKIEISQIDSLERIEANAFDNLLNLSEILIQNTK.

In another embodiment, the LHR fragment is the N-terminal of LHR protein with the following amino acid sequence:

RALREALCPEPCNCVPDGALRCPGPTAGLTRLSLAYLPVKVIPSQAFRGL
NEVIKIEISQIDSLERIEANAFDNLLNLSEILIQNTKNLRYIEPGAFINL
PRLKYLSICNTGIRKFPDVTKVFSSESNFILEICDNLHITTIPGNAFQGM
NNESVTLKLYGNGFEEVQSHAFNGTTLTSLELKENVHLEKMHNGAFRGAT
GPKTLDISSTKLQALPSYGLESIQRLIATSSYSLKKLPSRETFVNLLEAT
LTYPS.

In another embodiment, the antibody is a monoclonal antibody comprising an anti-LHR heavy chain variable region comprising, or alternatively consisting essentially of, or yet further consisting of a polypeptide selected from those disclosed herein or an equivalent of each thereof, and an anti-LHR light chain variable region comprising, or alternatively consisting essentially of, or yet further consisting of a polypeptide selected from those disclosed herein or an equivalent of each thereof.

In another aspect, the antibody is a chimeric antibody or a humanized antibody.

In some embodiments, the disclosed antibodies possess a binding affinity of at least 10⁻⁶ M. In certain aspects, antibodies bind with affinities of at least about 10⁻⁻⁷ M, and preferably 10⁻⁸ M, 10⁻⁹ M, 10⁻¹⁰ M, 10⁻¹¹ M, or 10⁻¹² M.

In another aspect, the heavy chain variable region comprises, or alternatively consists essentially of, or yet further consists of a polypeptide with a consensus sequence selected from disclosed herein, and an anti-LHR light chain variable region comprises, or alternatively consists essentially of, or yet further consists of a polypeptide with a consensus sequence selected from those disclosed herein.

In another aspect, the disclosure provides an isolated nucleic acid encoding the isolated anti-LHR antibody. In further embodiment, the isolated nucleic acid comprising, or alternatively consisting essentially of, or yet further consisting of a nucleic acid sequence selected from those disclosed herein, or an equivalent of each therefore.

In one aspect, the HC of the antibody comprises or alternatively consists essentially of, or yet further consists of one or more of: a CDR1 comprising the amino acid sequence of GYSITSGYG or an equivalent of each thereof; and/or a CDR2 comprising the amino acid sequence of IHYSGST or an equivalent of each thereof; and/or a CDR3 comprising the amino acid sequence of ARSLRY or an equivalent of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable regions of the antibodies comprises or alternatively consists essentially of, or yet further consists of one or more of: a CDR1 comprising the amino acid sequence of SSVNY or an equivalent of each thereof; and/or a CDR2 comprising the amino acid sequence of DTS or an equivalent of each thereof; and/or a CDR3 comprising the amino acid sequence of HQWSSYPYT or an equivalent of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In one aspect, the HC of the antibody comprises, or alternatively consists essentially of, or yet further consists of one or more of a CDR1 comprising the amino acid sequence of GFSLTTYG or an equivalent of each thereof; and/or a CDR2 comprising the amino acid sequence of IWGDGST or an equivalent of each thereof; and/or a CDR3 comprising the amino acid sequence of AEGSSLFAY or an equivalent of each thereof; and/or the LC of the antibody comprises, or alternatively consists essentially of, or yet further consists of a CDR1 comprising the amino acid sequence of QSLLNSGNQKNY or an equivalent of each thereof; and/or a CDR2 comprising the amino acid sequence of WAS or an equivalent of each thereof; and/or a CDR3 comprising the amino acid sequence of QNDYSYPLT or an equivalent of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In another aspect, the HC of the antibody comprises, or alternatively consists essentially of, or yet further consists of one or more of: a CDR1 comprising the amino acid sequence of GYSFTGYY or an equivalent of each thereof; and/or a CDR2 comprising the amino acid sequence of IYPYNGVS or an equivalent of each thereof; and/or a CDR3 comprising the amino acid sequence of ARERGLYQLRAMDY or an equivalent of each thereof; and/or the LC of the antibody comprises, or alternatively consists essentially of, or yet further consists of a CDR1 comprising the amino acid sequence of QSISNN or an equivalent of each thereof; and/or a CDR2 comprising the amino acid sequence of NAS or an equivalent of each thereof; and/or a CDR3 comprising the amino acid sequence of QQSNSWPYT or an equivalent of each thereof.

In one aspect, the disclosure provides an isolated anti-LHR antibody which is generated against a LHR fragment.

In one embodiment, the LHR fragment against which the antibody is raised is part of LHR protein with the following amino acid sequence:

REALCPEPCNCVPDGALRCPGPTAGLTRLSLAYLPVKVIPSQAFRGLNEV
IKIEISQIDSLERIEANAFDNLLNLSEILIQNTK.

In another embodiment, the LHR fragment is the N-terminal of LHR protein with the following amino acid sequence:

RALREALCPEPCNCVPDGALRCPGPTAGLTRLSLAYLPVKVIPSQAFRGL
NEVIKIEISQIDSLERIEANAFDNLLNLSEILIQNTKNLRYIEPGAFINL
PRLKYLSICNTGIRKFPDVTKVFSSESNFILEICDNLHITTIPGNAFQGM
NNESVTLKLYGNGFEEVQSHAFNGTTLTSLELKENVHLEKMHNGAFRGAT
GPKTLDISSTKLQALPSYGLESIQRLIATSSYSLKKLPSRETFVNLLEAT
LTYPS.

In another embodiment, the antibody is a monoclonal antibody comprising an anti-LHR heavy chain variable region comprising, or alternatively consisting essentially of, or yet further consisting of a polypeptide selected from those disclosed herein or an equivalent of each thereof.

In another embodiment, the antibody is a monoclonal antibody comprising an anti-LHR light chain variable region comprising, or alternatively consisting essentially of, or yet further consisting of a polypeptide selected from those disclosed herein or an equivalent of each thereof.

In another aspect, the anti-LHR antibody is a chimeric antibody, human or a humanized antibody.

In another aspect, the heavy chain variable region comprises, or alternatively consists essentially of, or yet further consists of a polypeptide with a consensus sequence selected from those disclosed herein, and an anti-LHR light chain variable region comprises, or alternatively consists essentially of, or yet further consists of a polypeptide with a consensus sequence selected from those disclosed herein, or equivalents of each thereof.

In another aspect of the present technology, the isolated antibody includes one or more of the following characteristics:

(a) the light chain immunoglobulin variable domain sequence comprises one or more CDRs that are at least 85% identical to a CDR of a light chain variable domain of any of the disclosed light chain sequences;

(b) the heavy chain immunoglobulin variable domain sequence comprises one or more CDRs that are at least 85% identical to a CDR of a heavy chain variable domain of any of the disclosed heavy chain sequences;

(c) the light chain immunoglobulin variable domain sequence is at least 85% identical to a light chain variable domain of any of the disclosed light chain sequences;

(d) the HC immunoglobulin variable domain sequence is at least 85% identical to a heavy chain variable domain of any of the disclosed light chain sequences; and

(e) the antibody binds an epitope that overlaps with an epitope bound by any of the disclosed sequences.

In one aspect, the present disclosure provides an isolated antibody that is at least 85% identical to the anti-LHR antibodies, e.g., 5F4-21, 4A7-4, 8B7-3 or 138-2, as disclosed herein.

In some of the aspects of the antibodies provided herein, the antibody binds human LHR with a dissociation constant (K_{D}) of less than 10⁻⁴M, 10⁻⁵M, 10⁻⁶M, 10⁻⁷M, 10⁻⁸M, 10⁻⁹M, 10⁻¹⁰M, 10⁻¹¹M, or 10⁻¹²M. In some of the aspects of the antibodies provided herein, the antigen binding site specifically binds to human LHR.

### Anti-B7-H4 Antibodies

In one aspect, the present disclosure provides an isolated antibody comprising a heavy chain (HC) immunoglobulin variable domain sequence and a light chain (LC) immunoglobulin variable domain sequence, wherein the heavy chain and light chain immunoglobulin variable domain sequences form an antigen binding site that binds to an epitope of human B7-H4. In one aspect, the antibodies possess a binding affinity of at least 10⁻⁶M. In certain aspects, antibodies bind with affinities of at least about 10⁻⁷M, and preferably 10⁻⁸M, 10⁻⁹ M, 10⁻¹⁰M, 10⁻¹¹M, or 10⁻¹²M.

In some embodiments, the heavy chain variable region comprises a CDRH1 sequence comprising, or alternatively consisting essentially of, or yet further consisting of, an amino acid sequence beginning with GXTF followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRH1 sequence comprises, or alternatively consists essentially of, or yet further consisting of, an amino acid sequence beginning with any one of the following sequences: (i) GFTFSSFG, (ii) GFTFSSYG, (iii) GYTFTDY, or equivalents thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH2 sequence comprising, or alternatively consisting essentially of, or yet further consisting of, an amino acid sequence beginning with ISSXXXT followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRH2 sequence comprises, or alternatively consists essentially of, or yet further consists of, an amino acid sequence beginning with any one of the following sequences: (i) ISSGSSTL, (ii) ISSSNSTI, or equivalents thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the heavy chain variable region comprises a CDRH2 sequence comprising, or alternatively consisting essentially of, or yet further consisting of, an amino acid sequence beginning with INPNNGGT or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH3 sequence comprising, or alternatively consisting essentially of, or yet further consisting of, an amino acid sequence beginning with ARPXYY followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRH3 sequence comprises, or alternatively consists essentially of, or yet further consisting of, an amino acid sequence beginning with any one of the following sequences: (i) ARPLYYYGSVMDY, (ii) ARPYYYGSSYDY, or equivalents thereof, followed by followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises, or alternatively consists essentially of, or yet further consists of, the polypeptide encoded by the below noted polynucleotide sequences: GAGGTGCAGCTGGAGGAGTCTGGGGGAGGCTTAGTGCAGCCTGGAGGGTCCCGG AAACTCTCCTGTGCAGCCTCTGGATTCACTTTCAGTAGCTTTGGAATGCACTGGG TTCGTCAGGCTCCAGAGAAGGGGCTGGAGTGGGTCGCATACATTAGTAGTGGCA GTAGTACCCTCCACTATGCAGACACAGTGAAGGGCCGATTCACCATCTCCAGAG ACAATCCCAAGAACACCCTGTTCCTGCAAATGAAACTACCCTCACTATGCTATGG ACTACTGGGGTCAAGGAACCTCAGTCACCGTCTCCTC or an antigen binding fragment thereof or an equivalent of each thereof.

In some embodiments, the heavy chain variable region comprises, or alternatively consists essentially of, or yet further consists of, the amino acid sequence: EVQLEESGGGLVQPGGSRKLSCAASGFTFSSFGMHWVRQAPEKGLEWVAYISSGSST LHYADTVKGRFTISRDNPKNTLFLQMKLPSLCYGLLGSRNLSHRLL (B7-H4 5F6 Heavy Variable) or an antigen binding fragment thereof or an equivalent of each thereof.

In some embodiments, the heavy chain variable region comprises, or alternatively consists essentially of, or yet further consists of, the polypeptide encoded by the below noted polynucleotide sequences: GATGTGCAGCTGGTGGAGTCTGGGGGAGGTTTAGTGCAGCCTGGAGGGTCCCGG AAACTCTCCTGTGCAGCCTCTGGATTCACTTTCAGTAGCTATGGAATTCACTGGG TTCGTCAGGTTCCAGAGAAGGGGCTGGAGTGGGTCGCATTTATTAGTAGTAGCAA TTCTACCATCTACTATGCAGACACAGTGAAGGGCCGATTCACCATCTCCAGAGAC AATGCCGAGAACACCCTGTTCCTGCAAATGACCAGTCTAAGGTCTGAGGACACG GCCATGTATTACTGTGCAAGACCCCTTTACTACTATGGTAGCGTTATGGACTACT GGGGTCAAGGAACCTCTGTCACCGTCTCCTCA or an antigen binding fragment thereof or an equivalent of each thereof.

In some embodiments, the heavy chain variable region comprises, or alternatively consists essentially of, or yet further consists of, the amino acid sequence: DVQLVESGGGLVQPGGSRKLSCAASGFTFSSYGIHWVRQVPEKGLEWVAFISSSNSTI YYADTVKGRFTISRDNAENTLFLQMTSLRSEDTAMYYCARPLYYYGSVMDYWGQG TSVTVSS (B7-H4 #33-14 Heavy Variable) or an antigen binding fragment thereof or an equivalent of each thereof.

In some embodiments, the heavy chain variable region comprises, or alternatively consists essentially of, or yet further consists of, the polypeptide encoded by the below noted polynucleotide sequences: GAGGTCCAGCTGCAACAATCTGGACCTGAGCTGGTGAAGCCTGGGGCTTCAGTG AAGATATCCTGTAAGGCTTCTGGATACACGTTCACTGACTACTACATGAACTGGA TGAAGCAGAGCCATGGAAAGAGTCTTGAGTGGATTGGAGATATTAATCCTAACA ATGGTGGTACTAGCTACAACCAGAAGTTCAAGGGCAAGGCCACATTGACTGTAG ACAAGTCCTCCAGCACAGCCTACATGGAACTCCGCAGCCTGACATCTGAGGACT CTGCAGTCTATTACTGTGCAAGACCTTATTACTACGGTAGTAGCTACGACTACTG GGGCCAAGGCACCACTCTCACAGTCTCCTCA or an antigen binding fragment thereof or an equivalent of each thereof.

In some embodiments, the heavy chain variable region comprises, or alternatively consists essentially of, or yet further consists of, the amino acid sequence: EVQLQQSGPELVKPGASVKISCKASGYTFTDYYMNWMKQSHGKSLEWIGDINPNNG GTSYNQKFKGKATLTVDKSSSTAYMELRSLTSEDSAVYYCARPYYYGSSYDYWGQ GTTLTVS (B7-H4 #36-1 Heavy Variable) or an antigen binding fragment thereof or an equivalent of each thereof.

In some embodiments, the light chain variable region comprises a CDRL1 sequence comprising, or alternatively consisting essentially of, or yet further consisting of, an amino acid sequence beginning with QSIVHXNGTY followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRL1 sequence comprises, or alternatively consists essentially of, or yet further consists of, an amino acid sequence beginning with any one of the following sequences: (i) QSIVHRNGNTY, (ii) QSIVHSNGNTY, or equivalents thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the light chain variable region comprises a CDRL1 sequence comprising, or alternatively consisting essentially of, or yet further consisting of, an amino acid sequence beginning with ENIGSY or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL2 sequence comprising, or alternatively consisting essentially of, or yet further consisting of, an amino acid sequence beginning with KVS followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the light chain variable region comprises a CDRL2 sequence comprising, or alternatively consisting essentially of, or yet further consisting of, an amino acid sequence beginning with AAT or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL3 sequence comprising, or alternatively consisting essentially of, or yet further consisting of, an amino acid sequence beginning with FQGSXVPXT followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus. In further embodiments, the CDRL1 sequence comprises, or alternatively consists essentially of, or yet further consists of, an amino acid sequence beginning with any one of the following sequences: (i) FQGSYVPPT, (ii) FQGSHVPLT, or equivalents thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the light chain variable region comprises a CDRL3 sequence comprising, or alternatively consisting essentially of, or yet further consisting of, an amino acid sequence beginning with QHYYSTLVT or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises, or alternatively consists essentially of, or yet further consists of, the polypeptide encoded by the polynucleotide sequence: GACATTGTGATCACCCAAACTCCACTCTCCCTGCCTGTCAGTCTTGGAGATCAAG CCTCCATCTCTTGCAGATCTAGTCAGAGCATTGTACATAGGAATGGAAACACCTA TTTAGAATGGTACTTGCAGCAACCAGGCCAGTCTCCAAAGCTCCTGATCTACAAA GTTTCCAACCGATTTTCTGGGGTCCCAGACAGGTTCAGTGGCAGTGGATCAGGGA CAGATTTCACACTCAAGATCAGCAGAGTGGAGGCTGAAGATCTGGGAGTTTATT ACTGCTTTCAAGGTTCATATGTTCCTCCGACGTTCGGTGGAGGCACCAAGCTGGA AATCAAA or an antigen binding fragment thereof or an equivalent of each thereof.

In some embodiments, the light chain variable region comprises, or alternatively consists essentially of, or yet further consists of, the amino acid sequence: DIVITQTPLSLPVSLGDQASISCRSSQSIVHRNGNTYLEWYLQQPGQSPKLLIYKVSNR FSGVPDRFSGSGSGTDFTLKISRVEAEDLGVYYCFQGSYVPPTFGGGTKLEIK (B7-H4 5F6 Light Variable) or an antigen binding fragment thereof or an equivalent of each thereof.

In some embodiments, the light chain variable region comprises, or alternatively consists essentially of, or yet further consists of, the polypeptide encoded by the polynucleotide sequence: GATGTTTTGATGACCCAAACTCCACTCTCCCTGCCTGTCAGTCTTGGAGATCAAG CCTCCATCTCTTGCAGATCTAGTCAGAGCATTGTACATAGTAATGGAAACACCTA TTTAGAATGGTACCTGCAGAAACCAGGCCAGTCTCCAAAGCTCCTGATCTACAAA GTTTCCAACCGATTTTCTGGGGTCCCAGACAGGTTCAGTGGCAGTGGATCAGGGA CAGATTTCACACTCAAGATAAGTAGAGTGGAGGCTGAGGATCTGGGAGTTTATT ACTGCTTTCAAGGTTCACATGTTCCTCTCACGTTCGGTGCAGGGACCAAGCTGGA ACTGAAA or an antigen binding fragment thereof or an equivalent of each thereof.

In some embodiments, the light chain variable region comprises, or alternatively consists essentially of, or yet further consists of, the amino acid sequence: DVLMTQTPLSLPVSLGDQASISCRSSQSIVHSNGNTYLEWYLQKPGQSPKLLIYKVSN RFSGVPDRFSGSGSGTDFTLKISRVEAEDLGVYYCFQGSHVPLTFGAGTKLELK (B7-H4 #33-14 Light Variable) or an antigen binding fragment thereof or an equivalent of each thereof.

In some embodiments, the light chain variable region comprises, or alternatively consists essentially of, or yet further consists of, the polypeptide encoded by the polynucleotide sequence: GACATCCAGATGACTCAGTCTCCAGCTTCCCTGTCTGCATCTGTGGGAGAAACTG TCACCATCACATGTCGAGCAAGTGAAAATATTGGCAGTTATTTAGCATGGTATCA GCAGAAACAGGGAAAATCTCCTCAGCTCCTGGTCTATGCTGCAACACTCTTAGCA GATGGTGTGCCATCAAGGTTCAGTGGCAGTGGATCAGGCACACAGTTTTCTCTCA AGATCAACAGCCTGCAGTCTGAAGATGTTGCGAGATATTACTGTCAACATTATTA TAGTACTCTGGTCACGTTCGGTGCTGGGACCAAGCTGGAACTGAAA or an antigen binding fragment thereof or an equivalent of each thereof.

In some embodiments, the light chain variable region comprises, or alternatively consists essentially of, or yet further consists of, the amino acid sequence: DIQMTQSPASLSASVGETVTITCRASENIGSYLAWYQQKQGKSPQLLVYAATLLADG VPSRFSGSGSGTQFSLKINSLQSEDVARYYCQHYYSTLVTFGAGTKLELK (B7-H4 #36-1 Light Variable) or an antigen binding fragment thereof or an equivalent of each thereof.

In another aspect of the present technology, the isolated antibody includes one or more of the following characteristics:

(a) the light chain immunoglobulin variable domain sequence comprises one or more CDRs that are at least 85% identical to a CDR of a light chain variable domain of any of the disclosed light chain sequences;

(b) the heavy chain immunoglobulin variable domain sequence comprises one or more CDRs that are at least 85% identical to a CDR of a heavy chain variable domain of any of the disclosed heavy chain sequences;

(c) the light chain immunoglobulin variable domain sequence is at least 85% identical to a light chain variable domain of any of the disclosed light chain sequences;

(d) the HC immunoglobulin variable domain sequence is at least 85% identical to a heavy chain variable domain of any of the disclosed light chain sequences; and

(e) the antibody binds an epitope that overlaps with an epitope bound by any of the disclosed sequences.

In one aspect, the present disclosure provides an isolated antibody that is at least 85% identical to an antibody selected from the group consisting of B7H4 5F6, B7H4 #33-14, and B7H4 #36-1.

In a further aspect, the antibodies identified above possess a binding affinity of at least 10⁻⁶M. In certain aspects, antibodies bind with affinities of at least about 10⁻⁷M, and preferably 10⁻⁸M, 10⁻⁹M, 10⁻¹⁰M, 10⁻¹¹M or 10⁻¹²M.

In one aspect, the present disclosure provides an isolated antibody comprising the CDRs of B7H4 5F6. In one aspect, the present disclosure provides an isolated antibody that is at least 85% identical to B7H4 5F6.

In one aspect, the present disclosure provides an isolated antibody comprising the CDRs of B7H4 #33-14. In one aspect, the present disclosure provides an isolated antibody that is at least 85% identical to B7H4 #33-14.

In one aspect, the present disclosure provides an isolated antibody comprising the CDRs of B7H4 #36-1. In one aspect, the present disclosure provides an isolated antibody that is at least 85% identical to B7H4 #36-1.

In some aspects of the antibodies provided herein, the HC variable domain sequence comprises a variable domain sequence of B7H4 5F6 and the LC variable domain sequence comprises a variable domain sequence of B7H4 5F6.

In some aspects of the antibodies provided herein, the HC variable domain sequence comprises a variable domain sequence of B7H4 #33-14 and the LC variable domain sequence comprises a variable domain sequence of B7H4 #33-14.

In some aspects of the antibodies provided herein, the HC variable domain sequence comprises a variable domain sequence of B7H4 #36-1 and the LC variable domain sequence comprises a variable domain sequence of B7H4 #36-1.

In some of the aspects of the antibodies provided herein, the antibody binds human B7-H4 with a dissociation constant (K_{D}) of less than 10⁻⁴M, 10⁻⁵M, 10⁻⁶M, 10⁻⁷M, 10⁻⁸M, 10⁻⁹M, 10⁻¹⁰M, 10⁻¹¹M, or 10⁻¹²M. In some of the aspects of the antibodies provided herein, the antigen binding site specifically binds to human B7-H4.

In some aspects of the antibodies provided herein, the antibody binds to the epitope bound by B7H4 5F6, B7H4 #33-14, and B7H4 #36-1 antibodies.

In some aspects of the antibodies provided herein, the B7-H4-specific antibody competes for binding to human B7-H4 with B7H4 5F6, B7H4 #33-14, and B7H4 #36-1.

### Anti-HLA-G Antibodies

In one aspect, the present disclosure provides an isolated antibody comprising a heavy chain (HC) immunoglobulin variable domain sequence and a light chain (LC) immunoglobulin variable domain sequence, wherein the heavy chain and light chain immunoglobulin variable domain sequences form an antigen binding site that binds to an epitope of human HLA-G.

In some embodiments, the heavy chain variable region comprises a CDRH1 sequence comprising, or alternatively consisting essentially of, or yet further consisting of, an amino acid sequence beginning with any one of the following sequences: (i) GFNIKDTY, (ii) GFTFNTYA (, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH2 sequence comprising, or alternatively consisting essentially of, or yet further consisting of, an amino acid sequence beginning with any one of the following sequences: (i) IDPANGNT, (ii) IRSKSNNYAT, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH3 sequence comprising, or alternatively consisting essentially of, or yet further consisting of, an amino acid sequence beginning with any one of the following sequences: (i) ARSYYGGFAY, (ii) VRGGYWSFDV, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises, or alternatively consists essentially of, or yet further consists of, the polypeptide encoded by the below noted polynucleotide sequences: CAGGTGCAGCTGCAGGAGTCAGGGGCAGAGCTTGTGAAGCCAGGGGCCTCAGTC AAGTTGTCCTGCACAGCTTCTGGCTTCAACATTAAAGACACCTATATGCACTGGG TGAAGCAGAGGCCTGAACAGGGCCTGGAGTGGATTGGAAGGATTGATCCTGCGA ATGGTAATACTAAATATGACCCGAAGTTCCAGGGCAAGGCCACTATAACAGCAG ACACATCCTCCAACACAGCCTACCTGCAGCTCAGCAGCCTGACATCTGAGGACA CTGCCGTCTATTACTGTGCTAGGAGTTACTACGGGGGGTTTGCTTACTGGGGCCA AGGGACTCTGGTCACTGTCTCTGCA or an antigen binding fragment thereof or an equivalent of each thereof.

In some embodiments, the heavy chain variable region comprises, or alternatively consists essentially of, or yet further consists of, the amino acid sequence: QVQLQESGAELVKPGASVKLSCTASGFNIKDTYMHWVKQRPEQGLEWIGRIDPANG NTKYDPKFQGKATITADTSSNTAYLQLSSLTSEDTAVYYCARSYYGGFAYWGQGTL VTVSA (3H11 Heavy Variable Chain) or an antigen binding fragment thereof or an equivalent of each thereof.

In some embodiments, the heavy chain variable region comprises, or alternatively consists essentially of, or yet further consists of, the polypeptide encoded by the below noted polynucleotide sequences: GAGGTGCAGCTGCAGGAGTCTGGTGGAGGATTGGTGCAGCCTAAAGGATCATTG AAACTCTCATGTGCCGCCTTTGGTTTCACCTTCAATACCTATGCCATGCACTGGGT CCGCCAGGCTCCAGGAAAGGGTTTGGAATGGGTTGCTCGCATAAGAAGTAAAAG TAATAATTATGCAACATATTATGCCGATTCAGTGAAAGACAGATTCACCATCTCC AGAGATGATTCACAAAGCATGCTCTCTCTGCAAATGAACAACCTGAAAACTGAG GACACAGCCATTTATTACTGTGTGAGAGGGGGTTACTGGAGCTTCGATGTCTGGG GCGCAGGGACCACGGTCACCGTCTCCTCA or an antigen binding fragment thereof or an equivalent of each thereof.

In some embodiments, the heavy chain variable region comprises, or alternatively consists essentially of, or yet further consists of, the amino acid sequence: EVQLQESGGGLVQPKGSLKLSCAAFGFTFNTYAMHWVRQAPGKGLEWVARIRSKS NNYATYYADSVKDRFTISRDDSQSMLSLQMNNLKTEDTAIYYCVRGGYWSFDVWG AGTTVTVSS (HLA-G 4E3 Heavy Variable Chain) or an antigen binding fragment thereof or an equivalent of each thereof.

In some embodiments, the light chain variable region comprises a CDRL1 sequence comprising, or alternatively consisting essentially of, or yet further consisting of, an amino acid sequence beginning with any one of the following sequences: (i) KSVSTSGYSY, (ii) KSLLHSNGNTY, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL2 sequence comprising, or alternatively consisting essentially of, or yet further consisting of, an amino acid sequence beginning with LVS, or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the light chain variable region comprises a CDRL2 sequence comprising, or alternatively consisting essentially of, or yet further consisting of, an amino acid sequence beginning with RMS or an equivalent thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL3 sequence comprising, or alternatively consisting essentially of, or yet further consisting of, an amino acid sequence beginning with any one of the following sequences: (i) QHSRELPRT, (ii) MQHLEYPYT, or equivalent of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises, or alternatively consists essentially of, or yet further consists of, the polypeptide encoded by the polynucleotide sequence: GATATTGTGCTCACACAGTCTCCTGCTTCCTTAGCTGTATCTCTGGGGCAGAGGG CCACCATCTCATGCAGGGCCAGCAAAAGTGTCAGTACATCTGGCTATAGTTATAT GCACTGGTACCAACAGAAACCAGGACAGCCACCCAAACTCCTCATCTATCTTGTA TCCAACCTAGAATCTGGGGTCCCTGCCAGGTTCAGTGGCAGTGGGTCTGGGACAG ACTTCACCCTCAACATCCATCCTGTGGAGGAGGAGGATGCTGCAACCTATTACTG TCAGCACAGTAGGGAGCTTCCTCGGACGTTCGGTGGAGGCACCAAGCTGGAAAT CAAA or an antigen binding fragment thereof or an equivalent of each thereof.

In some embodiments, the light chain variable region comprises, or alternatively consists essentially of, or yet further consists of, the amino acid sequence: DIVLTQSPASLAVSLGQRATISCRASKSVSTSGYSYMHWYQQKPGQPPKLLIYLVSNL ESGVPARFSGSGSGTDFTLNIHPVEEEDAATYYCQHSRELPRTFGGGTKLEIK (3H11 Light Variable Chain) or an antigen binding fragment thereof or an equivalent of each thereof.

In some embodiments, the light chain variable region comprises, or alternatively consists essentially of, or yet further consists of, the polypeptide encoded by the polynucleotide sequence: GATATTGTGATCACACAGACTACACCCTCTGTACCTGTCACTCCTGGAGAGTCAG TATCCATCTCCTGTAGGTCTAGTAAGAGTCTCCTGCATAGTAATGGCAACACTTA CTTGTATTGGTTCCTGCAGAGGCCAGGCCAGTCTCCTCAGCTCCTGATATCTCGG ATGTCCAGCCTTGCCTCAGGAGTCCCAGACAGGTTCAGTGGCAGTGGGTCAGGA ACTGCTTTCACACTGAGAATCAGTAGAGTGGAGGCTGAGGATGTGGGTGTTTATT ACTGTATGCAACATCTAGAATATCCGTATACGTTCGGAGGGGGGACCAAGCTGG AAATAAAA or an antigen binding fragment thereof or an equivalent of each thereof.

In some embodiments, the light chain variable region comprises, or alternatively consists essentially of, or yet further consists of, the amino acid sequence: DIVITQTTPSVPVTPGESVSISCRSSKSLLHSNGNTYLYWFLQRPGQSPQLLISRMSSLA SGVPDRFSGSGSGTAFTLRISRVEAEDVGVYYCMQHLEYPYTFGGGTKLEIK (HLA-G 4E3 Light Variable Chain) or an antigen binding fragment thereof or an equivalent of each thereof.

In another aspect of the present technology, the isolated antibody includes one or more of the following characteristics:

(a) the light chain immunoglobulin variable domain sequence comprises one or more CDRs that are at least 85% identical to a CDR of a light chain variable domain of any of the disclosed light chain sequences;

(b) the heavy chain immunoglobulin variable domain sequence comprises one or more CDRs that are at least 85% identical to a CDR of a heavy chain variable domain of any of the disclosed heavy chain sequences;

(c) the light chain immunoglobulin variable domain sequence is at least 85% identical to a light chain variable domain of any of the disclosed light chain sequences;

(d) the HC immunoglobulin variable domain sequence is at least 85% identical to a heavy chain variable domain of any of the disclosed light chain sequences; and

(e) the antibody binds an epitope that overlaps with an epitope bound by any of the disclosed sequences.

Exemplary antibodies comprising the disclosed CDR sequences and heavy and light chain variable sequences are disclosed in Table 1 and Table 2, respectively.

In one aspect, the present disclosure provides an isolated antibody that is at least 85% identical to an antibody selected from the group consisting of 3H11 and HLA-G 4E3.

In one aspect, the present disclosure provides an isolated antibody comprising the CDRs of 3H11. In one aspect, the present disclosure provides an isolated antibody that is at least 85% identical to 3H11.

In one aspect, the present disclosure provides an isolated antibody comprising the CDRs of HLA-G 4E3. In one aspect, the present disclosure provides an isolated antibody that is at least 85% identical to HLA-G 4E3.

In some aspects of the antibodies provided herein, the HC variable domain sequence comprises a variable domain sequence of 3H11 and the LC variable domain sequence comprises a variable domain sequence of 3H11.

In some aspects of the antibodies provided herein, the HC variable domain sequence comprises a variable domain sequence of HLA-G 4E3 and the LC variable domain sequence comprises a variable domain sequence of HLA-G 4E3.

In some of the aspects of the antibodies provided herein, the antibody binds human HLA-G with a dissociation constant (K_{D}) of less than 10⁻⁴M, 10⁻⁵M, 10⁻⁶M, 10⁻⁷M, 10⁻⁸M, 10⁻⁹M, 10⁻¹⁰M, 10⁻¹¹M, or 10⁻¹²M. In some of the aspects of the antibodies provided herein, the antigen binding site specifically binds to human HLA-G.

### Anti-HLA-DR Antibodies

In some embodiments, the heavy chain variable region comprises a CDRH1 sequence comprising, or alternatively consisting essentially of, or yet further consisting of, an amino acid sequence beginning with any one of the following sequences (i) GFSLTSYG, (ii) GFTFSNYW, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH2 sequence comprising, or alternatively consisting essentially of, or yet further consisting of, an amino acid sequence beginning with any one of the following sequences: (i) IWSDGST, (ii) IRFKSHNYAT, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH3 sequence comprising, or alternatively consisting essentially of, or yet further consisting of, an amino acid sequence beginning with any one of the following sequences: (i) ASHYGSTLAFAS, (ii) TRRIGNSDYDWWYFDV, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises, or alternatively consists essentially of, or yet further consists of, the polypeptide encoded by the below noted polynucleotide sequence: CAGGTGCAGCTGAAGGAGTCAGGACCTGGCCTGGTGGCGCCCTCACAGAGCCTG TCCATCACATGCACCATCTCAGGGTTCTCATTAACCAGCTATGGTGTACACTGGG TTCGCCAGCCTCCAGGAAAGGGTCTGGAGTGGCTGGTAGTGATATGGAGTGATG GAAGCACAACCTATAATTCAGCTCTCAAATCCAGACTGAGCATCAGCAAGGACA ACTCCAAGAGCCAAGTTTTCTTAAAAATGAACAGTCTCCAAACTGATGACACAGC CATATACTACTGTGCCAGTCACTACGGTAGTACCCTTGCCTTTGCTTCCTGGGGCC ACGGGACTCTGGTCACTGTCTCTGCA (Lym-1 Heavy Variable Chain),or an antigen binding fragment thereof or an equivalent of each thereof.

In some embodiments, the heavy chain variable region comprises, or alternatively consists essentially of, or yet further consists of, the amino acid sequence: QLKESGPGLVAPSQSLSITCTISGFSLTSYGVHWVRQPPGKGLEWLVVIWSDGSTTYN SALKSRLSISKDNSKSQVFLKMNSLQTDDTAIYYCASHYGSTLAFASWGHGTLVTVS A (Lym-1 Heavy Variable Chain), or an antigen binding fragment thereof or an equivalent of each thereof.

In some embodiments, the heavy chain variable region comprises, or alternatively consists essentially of, or yet further consists of, the polypeptide encoded by the below noted polynucleotide sequence: GAAGTGCAGCTTGAGGAGTCTGGAGGAGGCTTGGTGCAACCTGGAGGCTCCATG AAACTCTCCTGTGTTGCCTCTGGATTCACTTTCAGTAACTATTGGATGAACTGGGT CCGCCAGTCTCCAGAGAAGGGGCTTGAGTGGGTTGCTGAAATTAGATTTAAATCT CATAATTATGCAACACATTTTGCGGAGTCTGTGAAAGGGAGGTTCACCATCTCAA GAGATGATTCCAAAAGTAGTGTCTACCTGCAAATGAACAACTTAAGAGCTGAAG ACACTGGCATTTATTACTGTACCAGGAGGATAGGAAACTCTGATTACGACTGGTG GTACTTCGATGTCTGGGGCGCAGGGACCTCAGTCACCGTCTCCTCAGCTAGC (Lym-2 Light Heavy Chain), or an antigen binding fragment thereof or an equivalent of each thereof.

In some embodiments, the heavy chain variable region comprises, or alternatively consists essentially of, or yet further consists of, the amino acid sequence: EVQLEESGGGLVQPGGSMKLSCVASGFTFSNYWMNWVRQSPEKGLEWVAEIRFKS HNYATHFAESVKGRFTISRDDSKSSVYLQMNNLRAEDTGIYYCTRRIGNSDYDWWY FDVWGAGTSVTVSSAS (Lym-2 Heavy Variable Chain), or an antigen binding fragment thereof or an equivalent of each thereof.

In some embodiments, the light chain variable region comprises a CDRL1 sequence comprising, or alternatively consisting essentially of, or yet further consisting of, an amino acid sequence beginning with any one of the following sequences (i) VNIYSY, (ii) QNVGNN, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL2 sequence comprising, or alternatively consisting essentially of, or yet further consisting of, an amino acid sequence beginning with (i) NAK, (ii) SAS, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In other embodiments, the light chain variable region comprises a CDRL3 sequence comprising, or alternatively consisting essentially of, or yet further consisting of, an amino acid sequence beginning (i) QHHYGTFT, (ii) QQYNTYPFT, or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises, or alternatively consists essentially of, or yet further consists of, the polypeptide encoded by the polynucleotide sequence: GACATCCAGATGACTCAGTCTCCAGCCTCCCTATCTGCATCTGTGGGAGAAACTG TCACCATCATATGTCGAGCAAGTGTGAATATTTACAGTTATTTAGCATGGTATCA GCAGAAACAGGGAAAATCTCCTCAGCTCCTGGTCTATAATGCCAAAATCTTAGCA GAAGGTGTGCCATCAAGGTTCAGTGGCAGTGGATCAGGCACACAGTTTTCTCTGA AGATCAACAGCCTGCAGCCTGAAGATTTTGGGAGTTATTACTGTCAACATCATTA TGGTACATTCACGTTCGGCTCGGGGACAAAGTTGGAAATAAAA (Lym-1 Light Variable Chain), or an antigen binding fragment thereof or an equivalent of each thereof.

In some embodiments, the light chain variable region comprises, or alternatively consists essentially of, or yet further consists of, the amino acid sequence: DIQMTQSPASLSASVGETVTIICRASVNIYSYLAWYQQKQGKSPQLLVYNAKILAEGV PSRFSGSGSGTQFSLKINSLQPEDFGSYYCQHHYGTFTFGSGTKLEIK (Lym-1 Light Variable Chain), or an antigen binding fragment thereof or an equivalent of each thereof.

In some embodiments, the light chain variable region comprises, or alternatively consists essentially of, or yet further consists of, the polypeptide encoded by the polynucleotide sequence: GACATTGTGATGACCCAGTCTCACAAATTCATGTCCACATCAGTAGGAGACAGG GTCAGCGTCACCTGCAAGGCCAGTCAGAATGTGGGTAATAATGTAGCCTGGTATC AACAGAAACCAGGGCAATCTCCTAAAGTACTGATTTACTCGGCATCCTACCGGTA CAGTGGAGTCCCTGATCGCTTCACAGGCAGTGGATCTGGGACAGATTTCACTCTC ACCATCAGTAATGTGCAGTCTGAAGACTTGGCAGAGTATTTCTGTCAGCAATATA ACACCTATCCATTCACGTTCGGCTCGGGGACAAAGTTGGAAATAAAA (Lym-2 Light Variable Chain), or an antigen binding fragment thereof or an equivalent of each thereof.

In some embodiments, the light chain variable region comprises, or alternatively consists essentially of, or yet further consists of, the amino acid sequence: DIVMTQSHKFMSTSVGDRVSVTCKASQNVGNNVAWYQQKPGQSPKVLIYSASYRY SGVPDRFTGSGSGTDFTLTISNVQSEDLAEYFCQQYNTYPFTFGSGTKLEIK (Lym-2 Light Variable Chain), or an antigen binding fragment thereof or an equivalent of each thereof.

In another aspect of the present technology, the isolated antibody includes one or more of the following characteristics:

(a) the light chain immunoglobulin variable domain sequence comprises one or more CDRs that are at least 85% identical to a CDR of a light chain variable domain of any of the disclosed light chain sequences;

(b) the heavy chain immunoglobulin variable domain sequence comprises one or more CDRs that are at least 85% identical to a CDR of a heavy chain variable domain of any of the disclosed heavy chain sequences;

(c) the light chain immunoglobulin variable domain sequence is at least 85% identical to a light chain variable domain of any of the disclosed light chain sequences;

(d) the HC immunoglobulin variable domain sequence is at least 85% identical to a heavy chain variable domain of any of the disclosed light chain sequences; and

(e) the antibody binds an epitope that overlaps with an epitope bound by any of the disclosed sequences.

In one aspect, the present disclosure provides an isolated antibody that is at least 85% identical to an antibody selected from the group consisting of Lym-1 and Lym-2.

In one aspect, the present disclosure provides an isolated antibody comprising the CDRs of Lym-1. In one aspect, the present disclosure provides an isolated antibody that is at least 85% identical to Lym-1.

In one aspect, the present disclosure provides an isolated antibody comprising the CDRs of Lym-2. In one aspect, the present disclosure provides an isolated antibody that is at least 85% identical to Lym-2.

In some aspects of the antibodies provided herein, the HC variable domain sequence comprises, or consists essentially of, or yet further consists of, a variable domain sequence of Lym-1 and the LC variable domain sequence comprises, or consists essentially of, or yet further consists of a variable domain sequence of Lym-1.

In some aspects of the antibodies provided herein, the HC variable domain sequence comprises, or consists essentially of, or yet further consists of, a variable domain sequence of Lym-2 and the LC variable domain sequence comprises, or consists essentially of, or yet further consists of a variable domain sequence of Lym-2.

In some of the aspects of the antibodies provided herein, the antibody binds human HLA-DR with a dissociation constant (K_{D}) of less than 10⁻⁴M, 10⁻⁵M, 10⁻⁶M, 10⁻⁷M, 10⁻⁸M, 10⁻⁹M, 10⁻¹⁰M, 10⁻¹¹M, or 10⁻¹²M. In some of the aspects of the antibodies provided herein, the antigen binding site specifically binds to human HLA-DR.

### Antibody Features and Functions

In some of the aspects of the antibodies provided herein, the antibody is soluble Fab.

In some of the aspects of the antibodies provided herein, the HC and LC variable domain sequences are components of the same polypeptide chain. In some of the aspects of the antibodies provided herein, the HC and LC variable domain sequences are components of different polypeptide chains.

In some of the aspects of the antibodies provided herein, the antibody is a full-length antibody. In other aspect, antigen binding fragments of the antibodies are provided.

In some of the aspects of the antibodies provided herein, the antibody is a monoclonal antibody.

In some of the aspects of the antibodies provided herein, the antibody is chimeric or humanized.

In some of the aspects of the antibodies provided herein, the antibody fragment is selected from the group consisting of Fab, F(ab)′2, Fab′, scFᵥ, and Fᵥ.

In some of the aspects of the antibodies provided herein, the antibody antibody comprises an Fc domain. In some of the aspects of the antibodies provided herein, the antibody is a rabbit antibody. In some of the aspects of the antibodies provided herein, the antibody is a human or humanized antibody or is non-immunogenic in a human. In some of the aspects of the antibodies provided herein comprise a human antibody framework region.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families:

1) Amino acids with basic side chains: lysine, arginine, histidine;

2) Amino acids with acidic side chains: aspartic acid, glutamic acid;

3) Amino acids with uncharged polar side chains: asparagine, glutamine, serine, threonine, tyrosine;

4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies of the present disclosure comprising such varied CDR sequences still bind LHR, B7-H4, HLA-G, or HLA-DR with similar specificity and sensitivity profiles as the disclosed antibodies. This may be tested by way of the binding assays.

The constant regions of antibodies may also be varied. For example, antibodies may be provided with Fc regions of any isotype: IgA (IgA1, IgA2), IgD, IgE, IgG (IgG1, IgG2, IgG3, IgG4) or IgM. Non-limiting examples of constant region sequences include:

Human IgD constant region, Uniprot: P01880
APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK
Human IgG1 constant region, Uniprot: P01857
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Human IgG2 constant region, Uniprot: P01859
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK
Human IgG3 constant region, Uniprot: P01860
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK
Human IgM constant region, Uniprot: P01871
GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY
Human IgG4 constant region, Uniprot: P01861
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
Human IgA1 constant region, Uniprot: P01876
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY
Human IgA2 constant region, Uniprot: P01877
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRMAGKPTHVNVSVVMAEVDGTCY
Human Ig kappa constant region, Uniprot: P01834
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC

In some aspects, the antibodies comprise a heavy chain constant region that is at least 80% identical to any one of those disclosed herein.

In some aspects, the antibodies comprise a light chain constant region that is at least 80% identical to any one of those disclosed herein.

In some aspects of the antibodies provided herein, the antibody contains structural modifications to facilitate rapid binding and cell uptake and/or slow release. In some aspects, the LHR, B7-H4, HLA-G, or HLA-DR antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

The antibodies, fragments, and equivalents thereof can be combined with a carrier, e.g., a pharmaceutically acceptable carrier or other agents to provide a formulation for use and/or storage.

Further provided is an isolated polypeptide comprising, or alternatively consisting essentially of, or yet further consisting of, the amino acid sequence of LHR, B7-H4, HLA-G, or HLA-DR or a fragment thereof, that are useful to generate antibodies that bind to LHR, B7-H4, HLA-G, or HLA-DR, as well as isolated polynucleotides that encode them. In one aspect, the isolated polypeptides or polynucleotides further comprise a label or selection marker and/or contiguous polypeptide sequences (e.g., keyhole limpet haemocyanin (KLH) carrier protein) or in the case of polynucleotides, polynucleotides encoding the sequence, operatively coupled to polypeptide or polynucleotide. The polypeptides or polynucleotides can be combined with various carriers, e.g., phosphate buffered saline. Further provided are host cells, e.g., prokaryotic or eukaryotic cells, e.g., bacteria, yeast, mammalian (rat, simian, hamster, or human), comprising the isolated polypeptides or polynucleotides. The host cells can be combined with a carrier.

Yet further provided are the isolated nucleic acids encoding the antibodies and fragments thereof as disclosed herein. They can be combined with a vector or appropriate host cell, and/or a suitable carrier for diagnostic or therapeutic use. In one aspect, the nucleic acids are contained with a host cell for recombinant production of polypeptides and proteins. The host cells can be eukaryotic or prokaryotic.

II. Processes for Preparing Compositions

Antibodies, their manufacture and uses are well known and disclosed in, for example, Harlow, E. and Lane, D., Antibodies: A Laboratory Manual, Cold Spring Harbor Laboratory Press, Cold Spring Harbor, N.Y., 1999. The antibodies may be generated using standard methods known in the art. Examples of antibodies include (but are not limited to) monoclonal, single chain, and functional fragments of antibodies. Methods for generating such antibodies are known in the art; see, e.g. Collarini et al. (2009) J. Immunol. 183(10):6338-6345.

Antibodies may be produced in a range of hosts, for example goats, rabbits, rats, mice, humans, and others. They may be immunized by injection with a target antigen or a fragment or oligopeptide thereof which has immunogenic properties, such as a C-terminal fragment of LHR or B-7-H4, HLA-G, or HLA-DR an isolated polypeptide thereof. Depending on the host species, various adjuvants may be added and used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. This this disclosure also provides the isolated polypeptide and an adjuvant.

In certain aspects, the antibodies of the present disclosure are polyclonal, i.e., a mixture of plural types of anti-LHR, B7-H4, HLA-G, or HLA-DR antibodies having different amino acid sequences. In one aspect, the polyclonal antibody comprises a mixture of plural types of anti-LHR, B7-H4, HLA-G, or HLA-DR antibodies having different CDRs. As such, a mixture of cells which produce different antibodies is cultured, and an antibody purified from the resulting culture can be used (see WO 2004/061104).

Monoclonal Antibody Production. Monoclonal antibodies to LHR, B7-H4, HLA-G, or HLA-DR may be prepared using any technique which provides for the production of antibody molecules by continuous cell lines in culture. Such techniques include, but are not limited to, the hybridoma technique (see, e.g., Kohler & Milstein, Nature 256: 495-497 (1975)); the trioma technique; the human B-cell hybridoma technique (see, e.g., Kozbor, et al., Immunol. Today 4: 72 (1983)) and the EBV hybridoma technique to produce human monoclonal antibodies (see, e.g., Cole, et al., in: MONOCLONAL ANTIBODIES AND CANCER THERAPY, Alan R. Liss, Inc., pp. 77-96 (1985)). Human monoclonal antibodies can be utilized in the practice of the present technology and can be produced by using human hybridomas (see, e.g., Cote, et al., Proc. Natl. Acad. Sci. 80: 2026-2030 (1983)) or by transforming human B-cells with Epstein Barr Virus in vitro (see, e.g., Cole, et al., in: MONOCLONAL ANTIBODIES AND CANCER THERAPY, Alan R. Liss, Inc., pp. 77-96 (1985)). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the LHR, B7-H4, HLA-G, or HLA-DR polypeptide. Alternatively, hybridomas expressing anti-LHR, B7-H4, HLA-G, or HLA-DR monoclonal antibodies can be prepared by immunizing a subject, e.g., with an isolated polypeptide comprising, or alternatively consisting essentially of, or yet further consisting of, the amino acid sequence of LHR, B7-H4, HLA-G, or HLA-DR or a fragment thereof, and then isolating hybridomas from the subject's spleen using routine methods. See, e.g., Milstein et al., (Galfre and Milstein, Methods Enzymol 73: 3-46 (1981)). Screening the hybridomas using standard methods will produce monoclonal antibodies of varying specificity (i.e., for different epitopes) and affinity. A selected monoclonal antibody with the desired properties, e.g., LHR, B7-H4, HLA-G, or HLA-DR binding, can be (i) used as expressed by the hybridoma, (ii) bound to a molecule such as polyethylene glycol (PEG) to alter its properties, or (iii) a cDNA encoding the monoclonal antibody can be isolated, sequenced and manipulated in various ways. In one aspect, the anti-LHR, B7-H4, HLA-G, or HLA-DR monoclonal antibody is produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981).

Phage Display Technique. As noted above, the antibodies of the present disclosure can be produced through the application of recombinant DNA and phage display technology. For example, anti-LHR, B7-H4, HLA-G, or HLA-DR antibodies, can be prepared using various phage display methods known in the art. In phage display methods, functional antibody domains are displayed on the surface of a phage particle which carries polynucleotide sequences encoding them. Phage with a desired binding property is selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fᵥ or disulfide stabilized Fᵥ antibody domains are recombinantly fused to either the phage gene III or gene VIII protein. In addition, methods can be adapted for the construction of Fab expression libraries (see, e.g., Huse, et al., Science 246: 1275-1281, 1989) to allow rapid and effective identification of monoclonal Fab fragments with the desired specificity for a LHR, B7-H4, HLA-G, or HLA-DR polypeptide, e.g., a polypeptide or derivatives, fragments, analogs or homologs thereof. Other examples of phage display methods that can be used to make the isolated antibodies of the present disclosure include those disclosed in Huston et al., Proc. Natl. Acad. Sci. U.S.A., 85: 5879-5883 (1988); Chaudhary et al., Proc. Natl. Acad. Sci. U.S.A., 87: 1066-1070 (1990); Brinkman et al., J. Immunol. Methods 182: 41-50 (1995); Ames et al., J. Immunol. Methods 184: 177-186 (1995); Kettleborough et al., Eur. J. Immunol. 24: 952-958 (1994); Persic et al., Gene 187: 9-18 (1997); Burton et al., Advances in Immunology 57: 191-280 (1994); PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869 (1992); Sawai et al., AJRI 34: 26-34 (1995); and Better et al., Science 240: 1041-1043 (1988).

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See e.g. Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Alternate Methods of Antibody Production. Antibodies may also be produced by inducing in vivo production in the lymphocyte population or by screening recombinant immunoglobulin libraries or panels of highly specific binding reagents (Orlandi et al., PNAS 86: 3833-3837 (1989); Winter, G. et al., Nature, 349: 293-299 (1991)).

Alternatively, techniques for the production of single chain antibodies may be used. Single chain antibodies (scFᵥs) comprise a heavy chain variable region and a light chain variable region connected with a linker peptide (typically around 5 to 25 amino acids in length). In the scFᵥ, the variable regions of the heavy chain and the light chain may be derived from the same antibody or different antibodies. scFᵥs may be synthesized using recombinant techniques, for example by expression of a vector encoding the scFᵥ in a host organism such as E. coli. DNA encoding scFᵥ can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFᵥ and a host transformed by the expression vector can be obtained according to conventional methods known in the art.

Antigen binding fragments may also be generated, for example the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., Science, 256: 1275-1281 (1989)).

Antibody Modifications. The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFᵥ molecules, binding to streptavidin, introduction of a helix-turn-helix motif and the like can be exemplified.

The antibody compositions disclosed herein may be in the form of a conjugate formed between any of these antibodies and another agent (immunoconjugate). In one aspect, the antibodies disclosed herein are conjugated to radioactive material. In another aspect, the antibodies disclosed herein can be bound to various types of molecules such as polyethylene glycol (PEG).

Antibody Screening. Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between LHR, B7-H4, HLA-G, or HLA-DR, or any fragment or oligopeptide thereof and its specific antibody. A two-site, monoclonal-based immunoassay utilizing monoclonal antibodies specific to two non-interfering LHR, B7-H4, HLA-G, or HLA-DR epitopes may be used, but a competitive binding assay may also be employed (Maddox et al., J. Exp. Med., 158: 1211-1216 (1983)).

Automated immunohistochemistry (IHC) screening of potential anti-LHR, B7-H4, HLA-G, or HLA-DR antibodies can be performed using a Ventana Medical Systems, Inc (VMSI) Discovery XT and formalin-fixed, paraffin-embedded human tissue on glass slides. Tissue samples first undergo deparaffinization, antigen retrieval, followed by the addition of the potential anti-LHR, B7-H4, HLA-G, or HLA-DR antibody and a detection antibody. The detection antibody is visualized using a chromogen detection reagent from VMSI. Stained slides are manually screened under a microscope. Samples having a correct primary antibody staining pattern are selected as potential anti-LHR, B7-H4, HLA-G, or HLA-DR candidates.

Antibody Purification. The antibodies disclosed herein can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration, salt precipitation, dialysis, preparative polyacrylamide gel electrophoresis, isoelectric focusing electrophoresis, and the like. Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988).

Examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

In one aspect, a Protein A column or a Protein G column may be used in affinity chromatography. Other exemplary columns include a Protein A column, Hyper D, POROS, Sepharose F. F. (Pharmacia) and the like.

III. Methods of Use

General. The antibodies disclosed herein are useful in methods known in the art relating to the localization and/or quantitation of a LHR, B7-H4, HLA-G, or HLA-DR polypeptide (e.g., for use in measuring levels of the LHR, B7-H4, HLA-G, or HLA-DR polypeptide within appropriate physiological samples, for use in diagnostic methods, for use in imaging the polypeptide, and the like). The antibodies disclosed herein are useful in isolating a LHR, B7-H4, HLA-G, or HLA-DR polypeptide by standard techniques, such as affinity chromatography or immunoprecipitation. A LHR, B7-H4, HLA-G, or HLA-DR antibody disclosed herein can facilitate the purification of natural LHR, B7-H4, HLA-G, or HLA-DR polypeptides from biological samples, e.g., mammalian sera or cells as well as recombinantly-produced LHR, B7-H4, HLA-G, or HLA-DR polypeptides expressed in a host system. Moreover, LHR, B7-H4, HLA-G, or HLA-DR antibody can be used to detect a LHR, B7-H4, HLA-G, or HLA-DR polypeptide (e.g., in plasma, a cellular lysate or cell supernatant) in order to evaluate the abundance and pattern of expression of the polypeptide. The LHR, B7-H4, HLA-G, or HLA-DR antibodies disclosed herein can be used diagnostically to monitor LHR, B7-H4, HLA-G, or HLA-DR levels in tissue as part of a clinical testing procedure, e.g., to determine the efficacy of a given treatment regimen. The detection can be facilitated by coupling (i.e., physically linking) the LHR, B7-H4, HLA-G, or HLA-DR antibodies disclosed herein to a detectable substance.

In another aspect, provided herein is a composition comprising an antibody or antigen binding fragment as disclosed herein bound to a peptide comprising, for example, a human LHR, B7-H4, HLA-G, or HLA-DR protein or a fragment thereof. In one aspect, the peptide is associated with a cell. For example, the composition may comprise a disaggregated cell sample labeled with an antibody or antibody fragment as disclosed herein, which composition is useful in, for example, affinity chromatography methods for isolating cells or for flow cytometry-based cellular analysis or cell sorting. As another example, the composition may comprise a fixed tissue sample or cell smear labeled with an antibody or antibody fragment as disclosed herein, which composition is useful in, for example, immunohistochemistry or cytology analysis. In another aspect, the antibody or the antibody fragment is bound to a solid support, which is useful in, for example: ELISAs; affinity chromatography or immunoprecipitation methods for isolating LHR, B7-H4, HLA-G, or HLA-DR proteins or fragments thereof, LHR, B7-H4, HLA-G, or HLA-DR-positive cells, or complexes containing LHR, B7-H4, HLA-G, or HLA-DR and other cellular components. In another aspect, the peptide is bound to a solid support. For example, the peptide may be bound to the solid support via a secondary antibody specific for the peptide, which is useful in, for example, sandwich ELISAs. As another example, the peptide may be bound to a chromatography column, which is useful in, for example, isolation or purification of antibodies according to the present technology. In another aspect, the peptide is disposed in a solution, such as a lysis solution or a solution containing a sub-cellular fraction of a fractionated cell, which is useful in, for example, ELISAs and affinity chromatography or immunoprecipitation methods of isolating LHR, B7-H4, HLA-G, or HLA-DR proteins or fragments thereof or complexes containing LHR, B7-H4, HLA-G, or HLA-DR and other cellular components. In another aspect, the peptide is associated with a matrix, such as, for example, a gel electrophoresis gel or a matrix commonly used for western blotting (such as membranes made of nitrocellulose or polyvinylidene difluoride), which compositions are useful for electrophoretic and/or immunoblotting techniques, such as Western blotting.

Detection of LHR, B7-H4, HLA-G, or HLA-DR Polypeptides. An exemplary method for detecting the level of LHR, B7-H4, HLA-G, or HLA-DR polypeptides in a biological sample involves obtaining a biological sample from a subject and contacting the biological sample with a LHR, B7-H4, HLA-G, or HLA-DR antibody disclosed herein which is capable of detecting the LHR, B7-H4, HLA-G, or HLA-DR polypeptides.

In one aspect, the disclosed antibodies (e.g., 5F4-21, 4A7-4, 8B7-3, 138-2, B7-H4 5F6, B7-H4 #33-14, B7-H4 #36-1, HLA-G 4E3, 3H11, Lym-1, or Lym-2) or fragments thereof are detectably labeled. The term “labeled”, with regard to the antibody is intended to encompass direct labeling of the antibody by coupling (i.e., physically linking) a detectable substance to the antibody, as well as indirect labeling of the antibody by reactivity with another compound that is directly labeled. Non-limiting examples of indirect labeling include detection of a primary antibody using a fluorescently-labeled secondary antibody and end-labeling of a DNA probe with biotin such that it can be detected with fluorescently-labeled streptavidin.

The detection method of the present disclosure can be used to detect expression levels of LHR, B7-H4, HLA-G, or HLA-DR polypeptides in a biological sample in vitro as well as in vivo. In vitro techniques for detection of LHR, B7-H4, HLA-G, or HLA-DR polypeptides include enzyme linked immunosorbent assays (ELISAs), Western blots, flow cytometry, immunoprecipitations, radioimmunoassay, and immunofluorescence (e.g., IHC). Furthermore, in vivo techniques for detection of LHR, B7-H4, HLA-G, or HLA-DR polypeptides include introducing into a subject a labeled anti-LHR, B7-H4, HLA-G, or HLA-DR antibody. By way of example only, the antibody can be labeled with a radioactive marker whose presence and location in a subject can be detected by standard imaging techniques. In one aspect, the biological sample contains polypeptide molecules from the test subject.

Immunoassay and Imaging. A LHR, B7-H4, HLA-G, or HLA-DR antibody disclosed herein can be used to assay LHR, B7-H4, HLA-G, or HLA-DR polypeptide levels in a biological sample (e.g. human plasma) using antibody-based techniques. For example, protein expression in tissues can be studied with classical immunohistochemical (IHC) staining methods. Jalkanen, M. et al., J. Cell. Biol. 101: 976-985 (1985); Jalkanen, M. et al., J. Cell. Biol. 105: 3087-3096 (1987). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agents, such as iodine (¹²⁵I, ¹²¹I, ¹³¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying LHR, B7-H4, HLA-G, or HLA-DR polypeptide levels in a biological sample, LHR, B7-H4, HLA-G, or HLA-DR polypeptide levels can also be detected in vivo by imaging. Labels that can be incorporated with anti-LHR, B7-H4, HLA-G, or HLA-DR antibodies for in vivo imaging of LHR, B7-H4, HLA-G, or HLA-DR polypeptide levels include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the LHR, B7-H4, HLA-G, or HLA-DR antibody by labeling of nutrients for the relevant scFᵥ clone.

A LHR, B7-H4, HLA-G, or HLA-DR antibody which has been labeled with an appropriate detectable imaging moiety, such as a radioisotope (e.g.,¹³¹I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled LHR, B7-H4, HLA-G, or HLA-DR antibody will then preferentially accumulate at the location of cells which contain the specific target polypeptide. For example, in vivo tumor imaging is described in S. W. Burchiel et al., Tumor Imaging: The Radiochemical Detection of Cancer 13 (1982).

In some aspects, LHR, B7-H4, HLA-G, or HLA-DR antibodies containing structural modifications that facilitate rapid binding and cell uptake and/or slow release are useful in in vivo imaging detection methods. In some aspects, the LHR, B7-H4, HLA-G, or HLA-DR antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

Diagnostic Uses of LHR, B7-H4, HLA-G, or HLA-DR antibodies. The LHR, B7-H4, HLA-G, or HLA-DR antibody compositions disclosed herein are useful in diagnostic and prognostic methods. As such, the present disclosure provides methods for using the antibodies disclosed herein in the diagnosis of LHR, B7-H4, HLA-G, or HLA-DR-related medical conditions in a subject. Antibodies disclosed herein may be selected such that they have a high level of epitope binding specificity and high binding affinity to the LHR, B7-H4, HLA-G, or HLA-DR polypeptide. In general, the higher the binding affinity of an antibody, the more stringent wash conditions can be performed in an immunoassay to remove nonspecifically bound material without removing the target polypeptide. Accordingly, LHR, B7-H4, HLA-G, or HLA-DR antibodies of the present technology useful in diagnostic assays usually have binding affinities of at least 10⁻⁶, 10⁻⁷, 10⁻⁸, 10⁻⁹, 10⁻¹⁰, 10⁻¹¹, or 10⁻¹²M. In certain aspects, LHR, B7-H4, HLA-G, or HLA-DR antibodies used as diagnostic reagents have a sufficient kinetic on-rate to reach equilibrium under standard conditions in at least 12 hours, at least 5 hours, at least 1 hour, or at least 30 minutes.

Some methods of the present technology employ polyclonal preparations of anti-LHR, B7-H4, HLA-G, or HLA-DR antibodies and polyclonal anti-LHR, B7-H4, HLA-G, or HLA-DR antibody compositions as diagnostic reagents, and other methods employ monoclonal isolates. In methods employing polyclonal human anti-LHR, B7-H4, HLA-G, or HLA-DR antibodies prepared in accordance with the methods described above, the preparation typically contains an assortment of LHR, B7-H4, HLA-G, or HLA-DR antibodies, e.g., antibodies, with different epitope specificities to the target polypeptide. The monoclonal anti-LHR, B7-H4, HLA-G, or HLA-DR antibodies of the present disclosure are useful for detecting a single antigen in the presence or potential presence of closely related antigens.

The LHR, B7-H4, HLA-G, or HLA-DR antibodies of the present disclosure can be used as diagnostic reagents for any kind of biological sample. In one aspect, the LHR, B7-H4, HLA-G, or HLA-DR antibodies disclosed herein are useful as diagnostic reagents for human biological samples. LHR, B7-H4, HLA-G, or HLA-DR antibodies can be used to detect LHR, B7-H4, HLA-G, or HLA-DR polypeptides in a variety of standard assay formats. Such formats include immunoprecipitation, Western blotting, ELISA, radioimmunoassay, flow cytometry, IHC and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue (including biopsies), cell or body fluid of a subject.

Prognostic Uses of LHR, B7-H4, HLA-G, or HLA-DR antibodies. The present disclosure also provides for prognostic (or predictive) assays for determining whether a subject is at risk of developing a medical disease or condition associated with increased LHR, B7-H4, HLA-G, or HLA-DR polypeptide expression or activity (e.g., detection of a precancerous cell). Such assays can be used for prognostic or predictive purpose to thereby prophylactically treat an individual prior to the onset of a medical disease or condition characterized by or associated with LHR, B7-H4, HLA-G, or HLA-DR polypeptide expression.

Another aspect of the present disclosure provides methods for determining LHR, B7-H4, HLA-G, or HLA-DR expression in a subject to thereby select appropriate therapeutic or prophylactic compounds for that subject.

Alternatively, the prognostic assays can be utilized to identify a subject having or at risk for developing cancer and/or solid tumors. In certain embodiments, the cancer and/or tumor is of the thyroid, breast, colon, prostate, ovary or more specifically a chrio-carcinoma or the caner and/or tumor is a B-cell lymphoma or leukemia. Thus, the present disclosure provides a method for identifying a disease or condition associated with increased LHR, B7-H4, HLA-G, or HLA-DR polypeptide expression levels in which a test sample is obtained from a subject and the LHR, B7-H4, HLA-G, or HLA-DR polypeptide detected, wherein the presence of increased levels of LHR, B7-H4, HLA-G, or HLA-DR polypeptides compared to a control sample is predictive for a subject having or at risk of developing a disease or condition associated with increased LHR, B7-H4, HLA-G, or HLA-DR polypeptide expression levels. In some aspects, the disease or condition associated with increased LHR, B7-H4, HLA-G, or HLA-DR polypeptide expression levels is selected from the group consisting of cancer and/or solid tumors. In certain embodiments, the cancer and/or tumor is of the thyroid, breast, colon, prostate, ovary, or a chrio-carcinoma or a B-cell lymphoma or leukemia.

In another aspect, the present disclosure provides methods for determining whether a subject can be effectively treated with a compound for a disorder or condition associated with increased LHR, B7-H4, HLA-G, or HLA-DR polypeptide expression wherein a biological sample is obtained from the subject and the LHR, B7-H4, HLA-G, or HLA-DR polypeptide is detected using the LHR, B7-H4, HLA-G, or HLA-DR antibody. The expression level of the LHR, B7-H4, HLA-G, or HLA-DR polypeptide in the biological sample obtained from the subject is determined and compared with the LHR, B7-H4, HLA-G, or HLA-DR expression levels found in a biological sample obtained from a subject or Isolated from a patient population who is free of the disease. Elevated levels of the LHR, B7-H4, HLA-G, or HLA-DR polypeptide in the sample obtained from the subject suspected of having the disease or condition compared with the sample obtained from the healthy subject is indicative of the LHR, B7-H4, HLA-G, or HLA-DR-associated disease or condition in the subject being tested. Increased expression of the LHR, B7-H4, HLA-G, or HLA-DR polypeptide, as compared to the expression level of the polypeptide or protein in the patient sample(s) from the patients free of disease indicates that the patient is likely to be responsive to the CAR T cell or CAR NK cell therapy of this disclosure, and lack of elevated expression indicates that the patient is not likely to be responsive to the CAR T cell or CAR NK cell therapy. Non-limiting examples of samples include, e.g., any body fluid including, but not limited to, e.g., sputum, serum, plasma, lymph, cystic fluid, urine, stool, cerebrospinal fluid, ascite fluid or blood and including biopsy samples of body tissue. The samples are also a tumor cell. The test sample used in the above-described method will vary based on the assay format, nature of the detection method and the tissues, cells or extracts used as the sample to be assayed.

In a particular aspect, the present disclosure relates to methods for determining if a patient is likely to respond or is not likely to LHR, B7-H4, HLA-G, or HLA-DR CAR therapy. In specific embodiments, this method comprises contacting a tumor sample isolated from the patient with an effective amount of an LHR, B7-H4, HLA-G, or HLA-DR binding agent, e.g., an LHR, B7-H4, HLA-G, or HLA-DR antibody and detecting the presence of any agent or antibody bound to the tumor sample. In further embodiments, the presence of agent or antibody bound to the tumor sample indicates that the patient is likely to respond to the LHR, B7-H4, HLA-G, or HLA-DR CAR therapy and the absence of antibody bound to the tumor sample indicates that the patient is not likely to respond to the LHR, B7-H4, HLA-G, or HLA-DR therapy. Non-limiting examples of samples include, e.g., any body fluid including, but not limited to, e.g., sputum, serum, plasma, lymph, cystic fluid, urine, stool, cerebrospinal fluid, ascite fluid or blood and including biopsy samples of body tissue. The samples are also a tumor cell. The test sample used in the above-described method will vary based on the assay format, nature of the detection method and the tissues, cells or extracts used as the sample to be assayed. In some embodiments, the method comprises the additional step of administering an effective amount of the LHR, B7-H4, HLA-G, or HLA-DR CAR therapy to a patient that is determined likely to respond to the LHR, B7-H4, HLA-G, or HLA-DR CAR therapy. In some embodiments, the patient a LHR, B7-H4, HLA-G, or HLA-DR expressing tumor and/or cancer.

There are a number of disease states in which the elevated expression level of LHR, B7-H4, HLA-G, or HLA-DR polypeptides is known to be indicative of whether a subject with the disease is likely to respond to a particular type of therapy or treatment. Non-limiting examples of such disease states include cancer, e.g., a carcinoma, a sarcoma or a leukemia. Thus, the method of detecting a LHR, B7-H4, HLA-G, or HLA-DR polypeptide in a biological sample can be used as a method of prognosis, e.g., to evaluate the likelihood that the subject will respond to the therapy or treatment. The level of the LHR, B7-H4, HLA-G, or HLA-DR polypeptide in a suitable tissue or body fluid sample from the subject is determined and compared with a suitable control, e.g., the level in subjects with the same disease but who have responded favorably to the treatment. Non-limiting examples of samples include, e.g., any body fluid including, but not limited to, e.g., sputum, serum, plasma, lymph, cystic fluid, urine, stool, cerebrospinal fluid, ascite fluid or blood and including biopsy samples of body tissue. The samples are also a tumor cell. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

In one aspect, the present disclosure provides for methods of monitoring the influence of agents (e.g., drugs, compounds, or small molecules) on the expression of LHR, B7-H4, HLA-G, or HLA-DR polypeptides. Such assays can be applied in basic drug screening and in clinical trials. For example, the effectiveness of an agent to decrease LHR, B7-H4, HLA-G, or HLA-DR polypeptide levels can be monitored in clinical trials of subjects exhibiting elevated expression of LHR, B7-H4, HLA-G, or HLA-DR, e.g., patients diagnosed with cancer. An agent that affects the expression of LHR, B7-H4, HLA-G, or HLA-DR polypeptides can be identified by administering the agent and observing a response. In this way, the expression pattern of the LHR, B7-H4, HLA-G, or HLA-DR polypeptide can serve as a marker, indicative of the physiological response of the subject to the agent. Accordingly, this response state may be determined before, and at various points during, treatment of the subject with the agent. In some embodiments, the method further comprises the additional step of administering an effective amount of the LHR, B7-H4, HLA-G, or HLA-DR CAR therapy to a patient that is determined to require additional therapy.

Further aspects of the present disclosure relate to methods for determining if a patient is likely to respond or is not likely to LHR, B7-H4, HLA-G, or HLA-DR CAR therapy. In specific embodiments, this method comprises contacting a tumor sample isolated from the patient with an effective amount of an LHR, B7-H4, HLA-G, or HLA-DR antibody and detecting the presence of any antibody bound to the tumor sample. In further embodiments, the presence of antibody bound to the tumor sample indicates that the patient is likely to respond to the LHR, B7-H4, HLA-G, or HLA-DR CAR therapy and the absence of antibody bound to the tumor sample indicates that the patient is not likely to respond to the LHR, B7-H4, HLA-G, or HLA-DR therapy. In some embodiments, the method comprises the additional step of administering an effective amount of the LHR, B7-H4, HLA-G, or HLA-DR CAR therapy to a patient that is determined likely to respond to the LHR, B7-H4, HLA-G, or HLA-DR CAR therapy. In some embodiments, the patient a B7-H4 expressing tumor and/or cancer. In some embodiments, the tumor and/or cancer is a solid tumor, e.g., breast, colon, prostate, thyroid, or chorio-carcinoma. In some embodiments, the cancer/tumor is a B-cell lymphoma or leukemia.

Automated Embodiments. A person of ordinary skill in the art will appreciate that aspects of the methods for using the LHR, B7-H4, HLA-G, or HLA-DR antibodies disclosed herein can be automated. Particular aspects of LHR, B7-H4, HLA-G, or HLA-DR staining procedures can be conducted using various automated processes.

IV. Kits

As set forth herein, the present disclosure provides diagnostic methods for determining the expression level of LHR, B7-H4, HLA-G, or HLA-DR. In one particular aspect, the present disclosure provides kits for performing these methods as well as instructions for carrying out the methods of the present disclosure such as collecting tissue and/or performing the screen, and/or analyzing the results.

The kit comprises, or alternatively consists essentially of, or yet further consists of, a LHR, B7-H4, HLA-G, or HLA-DR antibody composition (e.g., monoclonal antibodies) disclosed herein, and instructions for use. The kits are useful for detecting the presence of LHR, B7-H4, HLA-G, or HLA-DR polypeptides in a biological sample e.g., any bodily fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

In some aspects, the kit can comprise: one or more LHR, B7-H4, HLA-G, or HLA-DR antibodies capable of binding a LHR, B7-H4, HLA-G, or HLA-DR polypeptide in a biological sample (e.g., an antibody or antigen-binding fragment thereof having the same antigen-binding specificity of LHR, B7-H4, HLA-G, or HLA-DR antibody B7H4 5F6, B7H4 #33-14, or B7H4 #36-1); means for determining the amount of the LHR, B7-H4, HLA-G, or HLA-DR polypeptide in the sample; and means for comparing the amount of the LHR, B7-H4, HLA-G, or HLA-DR polypeptide in the sample with a standard. One or more of the LHR, B7-H4, HLA-G, or HLA-DR antibodies may be labeled. The kit components, (e.g., reagents) can be packaged in a suitable container. The kit can further comprise instructions for using the kit to detect the LHR, B7-H4, HLA-G, or HLA-DR polypeptides. In certain aspects, the kit comprises a first antibody, e.g., attached to a solid support, which binds to a LHR, B7-H4, HLA-G, or HLA-DR polypeptide; and, optionally; 2) a second, different antibody which binds to either the LHR, B7-H4, HLA-G, or HLA-DR polypeptide or the first antibody and is conjugated to a detectable label.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

V. Carriers

The antibodies of the present disclosure also can be bound to many different carriers. Thus, this disclosure also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding antibodies, or will be able to ascertain such, using routine experimentation.

### Chimeric Antigen Receptors and Uses Thereof

I. Compositions

The present disclosure provides chimeric antigen receptors (CAR) that bind to LHR, B7-H4, HLA-G, or HLA-DR comprising, consisting, or consisting essentially of, a cell activation moiety comprising an extracellular, transmembrane, and intracellular domain. The extracellular domain comprises a target-specific binding element otherwise referred to as the antigen binding domain. The intracellular domain or cytoplasmic domain comprises, at least one costimulatory signaling region and a zeta chain portion.

Spacer Domain. The CAR may optionally further comprise a spacer domain of up to 300 amino acids, preferably 10 to 100 amino acids, more preferably 25 to 50 amino acids. For example, the spacer may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. A spacer domain may comprises, for example, a portion of a human Fc domain, a CH3 domain, or the hinge region of any immunoglobulin, such as IgA, IgD, IgE, IgG, or IgM, or variants thereof. For example, some embodiments may comprise an IgG4 hinge with or without a S228P, L235E, and/or N297Q mutation (according to Kabat numbering). Additional spacers include, but are not limited to, CD4, CD8, and CD28 hinge regions.

Antigen Binding Domain. In certain aspects, the present disclosure provides a CAR that comprises, consists, or alternatively consists essentially thereof of an antigen binding domain specific to LHR, B7-H4, HLA-G, or HLA-DR. In some embodiments, the antigen binding domain comprises, or alternatively consists essentially thereof, or yet consists of the antigen binding domain of an anti-LHR, B7-H4, HLA-G, or HLA-DR antibody. In further embodiments, the heavy chain variable region and light chain variable region of an anti-LHR, B7-H4, HLA-G, or HLA-DR antibody comprises, or alternatively consists essentially thereof, or yet consists of the antigen binding domain the anti-LHR, B7-H4, HLA-G, or HLA-DR antibody. In some embodiments, the antigen binding domain comprises, consists, or consists essentially of a fragment of the target-specific antibody (i.e. an anti-LHR, B7-H4, HLA-G, or HLA-DR antibody), for example, an scFv.

An scFv region can comprise the variable regions of the heavy (V_{H}) and light chains (V_{L}) of immunoglobulins, connected with a short linker peptide. The linker peptide may be from 1 to 50 amino acids, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine.

In some embodiments, the heavy chain variable region of the antibody comprises, or consists essentially thereof, or consists of those disclosed herein or an equivalent of each thereof and/or comprises one or more CDR regions comprising those disclosed herein or an equivalent of each thereof. In some embodiments, the light chain variable region of the antibody comprises, or consists essentially thereof, or consists of those disclosed herein or an equivalent of each thereof and/or comprises one or more CDR regions comprising those disclosed herein or an equivalent of each thereof.

Transmembrane Domain. The transmembrane domain may be derived either from a natural or from a synthetic source. Where the source is natural, the domain may be derived from any membrane-bound or transmembrane protein. Transmembrane regions of particular use in this disclosure may be derived from CD8, CD28, CD3, CD45, CD4, CDS, CDS, CD9, CD 16, CD22, CD33, CD37, CD64, CD80, CD86, CD 134, CD137, CD 154, TCR. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Cytoplasmic Domain. The cytoplasmic domain or intracellular signaling domain of the CAR is responsible for activation of at least one of the traditional effector functions of an immune cell in which a CAR has been placed. The intracellular signaling domain refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the T-cell receptor (TCR) and co-receptors, as well as derivatives or variants thereof, can function as intracellular signaling domains for use in a CAR. Intracellular signaling domains of particular use in this disclosure may be derived from FcR, TCR, CD3, CDS, CD22, CD79a, CD79b, CD66d. In some embodiments, the signaling domain of the CAR can comprise a CD3 signaling domain.

Since signals generated through the TCR are alone insufficient for full activation of a T cell, a secondary or co-stimulatory signal may also be required. Thus, the intracellular region of at least one co-stimulatory signaling molecule, including but not limited to CD27, CD28, 4-IBB (CD 137), OX40, CD30, CD40, PD-1, ICOS, lymphocyte function-associated antigen-1 (LFA-1), CD2, CD7, LIGHT, NKG2C, B7-H3, or a ligand that specifically binds with CD83, may also be included in the cytoplasmic domain of the CAR. CARs of the present disclosure can comprise one or more co-stimulatory domain. For instance, a CAR may comprise one, two, or more co-stimulatory domains, in addition to a signaling domain (e.g., a CD3 ζ signaling domain).

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, OX40, CD30, CD40, PD-1, ICOS, LFA-1, CD2, CD7, CD27, LIGHT, NKG2C, B7-H3 and CD3-zeta protein.

In specific embodiments, the CAR comprises, or alternatively consists essentially thereof, or yet consists of an antigen binding domain of an anti-LHR, B7-H4, HLA-G, or HLA-DR antibody or fragment (e.g., scFv) thereof, a CD8 α hinge domain, a CD8 α transmembrane domain, at least one costimulatory signaling region, and a CD3 zeta signaling domain. In further embodiments, the costimulatory signaling region comprises either or both a CD28 costimulatory signaling region and a 4-1BB costimulatory signaling region.

In some embodiments, the CAR can further comprise a detectable marker or purification marker.

Switch Mechanisms. In some embodiments, the CAR may also comprise a switch mechanism for controlling expression and/or activation of the CAR. For example, a CAR may comprise, consist, or consist essentially of an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises, consists, or consists essentially of a target antigen binding domain (e.g., an anti-LHR, B7-H4, HLA-G, or HLA-DR antibody or fragment thereof or a bispecific antibody that binds LHR, B7-H4, HLA-G, or HLA-DR and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but it cannot bind its target antigen (i.e., LHR, B7-H4, HLA-G, or HLA-DR) until the second composition comprising an LHR, B7-H4, HLA-G, or HLA-DR-specific binding domain is administered.

CARs of the present disclosure may likewise require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015) in order to elicit a T-cell response.

Furthermore, the disclosed CARs can comprise a “suicide switch” or “safety switch” to induce cell death of the CAR T-cells following treatment (Buddee et al., PLoS One, 2013) or to downregulate expression of the CAR following binding to the target antigen (WO 2016/011210). For example, CARs can be modified with a suicide gene that confers sensitivity to an antibody or prodrug that can be administered to cease CAR activity. In some embodiments, the antibody or prodrug is administered to a subject that has received CAR therapy upon the occurrence of an adverse event. Exemplary suicide genes include but are not limited to herpes simplex virus-thymidine kinase (HSV-TK) which renders cells susceptible to ganciclovir (Bonini et al. Science 276: 1719-1724 (1997)), inducible Caspase 9 which allows for dimerization and activation of apoptosis when activated by a dimerizer drug (Gargett et al., Front Pharmacol, 2014 5:235), and truncated EGFR which renders cells susceptible to cetuximab (Wang et al. Blood 118: 1255-63 (2011)).

In a further aspect, this disclosure provides complex comprising an HLA-DR CAR cell bound to its target cell. In a further aspect, the complex is detectably labeled. Detectable labels are known in the art and briefly described herein.

II. Process for Preparing CARs

Also provided herein is a method of producing LHR, B7-H4, HLA-G, or HLA-DRCAR expressing cells comprising, or alternatively consisting essentially of, or yet further consisting of the steps: (i) transducing a population of isolated cells with a nucleic acid sequence encoding the CAR as described herein; and (ii) selecting a subpopulation of said isolated cells that have been successfully transduced with said nucleic acid sequence of step (i) thereby producing LHR, B7-H4, HLA-G, or HLA-DRCAR expressing cells. In one aspect, the isolated cells are selected from a group consisting of T-cells and NK-cells.

Aspects of the present disclosure relate to an isolated cell comprising a LHR, B7-H4, HLA-G, or HLA-DR-specific CAR and methods of producing such cells. The cell is a prokaryotic or a eukaryotic cell. In one aspect, the cell is a T cell or an NK cell. The eukaryotic cell can be from any preferred species, e.g., an animal cell, a mammalian cell such as a human, a feline or a canine cell.

In some aspects of the present disclosure, the population of isolated cells transduced with the nucleic acid sequence encoding the CAR as described herein is a population of NK precursor cells and/or T-cell precursor cells. Transduction of precursor cells results in a long-lived population of cells capable of differentiating into CAR T-cells and/or CAR NK cells. T-cell precursors include but are not limited to HSCs; long term HSCs; MPPs; CLPs; LMPPs/ELPs; DN1s; DN2s; DN3s; DN4s; DPs. NK precursors include but are not limited to HSCs, long term HSCs, MPPs, CMPs, GMPs, pro-NK, pre-NK, and iNK cells. In a specific aspect, the population of isolated cells includes both mature T-cells and T-cell precursors to provide both short lived effector CAR T-cells and long lived CAR T-cell precursors for transplant into the subject. In another aspect, the population of isolated cells includes both mature NK cells and NK precursors to provide both short lived effector CAR NK cells and long lived CAR NK precursors for transplant into the subject.

In specific embodiments, the isolated cell comprises, or alternatively consists essentially of, or yet further consists of an exogenous CAR comprising, or alternatively consisting essentially of, or yet further consisting of, an antigen binding domain of an anti-LHR, B7-H4, HLA-G, or HLA-DR antibody, a CD8 α hinge domain, a CD8 α transmembrane domain, a CD28 costimulatory signaling region and/or a 4-1BB costimulatory signaling region, and a CD3 zeta signaling domain. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell.

In certain embodiments, methods of producing LHR, B7-H4, HLA-G, or HLA-DR CAR expressing cells are disclosed comprising, or alternatively consisting essentially of: (i) transducing a population of isolated cells with a nucleic acid sequence encoding a LHR, B7-H4, HLA-G, or HLA-DR CAR and (ii) selecting a subpopulation of cells that have been successfully transduced with said nucleic acid sequence of step (i). In some embodiments, the isolated cells are T-cells, an animal T-cell, a mammalian T-cell, a feline T-cell, a canine T-cell or a human T-cell, thereby producing LHR, B7-H4, HLA-G, or HLA-DR CAR T-cells. In certain embodiments, the isolated cell is an NK-cell, e.g., an animal NK-cell, a mammalian NK-cell, a feline NK-cell, a canine NK-cell or a human NK-cell, thereby producing LHR, B7-H4, HLA-G, or HLA-DR CAR NK-cells.

In some embodiments, T-cells expressing the disclosed CARs may be further modified to reduce or eliminate expression of endogenous TCRs. Reduction or elimination of endogenous TCRs can reduce off-target effects and increase the effectiveness of the T cells. T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T cell having a stable deficiency in functional TCR expression.

Expression of inhibitory RNAs (e.g., shRNA, siRNA, miRNA, etc.) in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the RNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

Expression of CRISPR in primary T cells can be achieved using conventional CRISPR/Cas sytems and guide RNAs specific to the target TCRs. Suitable expression systems, e.g. lentiviral or adenoviral expression systems are known in the art. Similar to the delivery of inhibitor RNAs, the CRISPR system can be use to specifically target resting primary T cells or other suitable immune cells for CAR cell therapy. Further, to the extent that CRISPR editing is unsuccessful, cells can be selected for success according to the methods disclosed above. For example, as noted above, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

Sources of Isolated Cells. Prior to expansion and genetic modification of the cells disclosed herein, cells may be obtained from a subject—for instance, in embodiments involving autologous therapy—or a commercially available culture, that are available from the American Type Culture Collection (ATCC), for example.

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include, but are not limited to Life Technologies Dynabeads® system; STEMcell Technologies EasySep™, RoboSep™ RosetteSep™, SepMate™; Miltenyi Biotec MACS™ cell separation kits, and other commercially available cell separation and isolation kits. Particular subpopulations of immune cells and precursors may be isolated through the use of fluorescence-activated cell sorting (FACS), beads, or other binding agents available in such kits specific to unique cell surface markers. For example, MACS™ CD4+ and CD8+ MicroBeads may be used to isolate CD4+ and CD8+ T-cells.

Alternatively, cells may be obtained through commercially available cell cultures, including but not limited to, for T-cells, lines BCL2 (AAA) Jurkat (ATCC® CRL-2902™) BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); and, for NK cells, lines NK-92 (ATCC® CRL-2407™), NK-92MI (ATCC® CRL-2408™).

In some aspects, the subject may be administered a conditioning regimen to induce precursor cell mobilization into the peripheral blood prior to obtaining the cells from the subject. For example, a subject may be administered an effective amount of at least one of granulocyte colony-stimulating factor (G-CSF), filgrastim (Neupogen), sargramostim (Leukine), pegfilgrastim (Neulasta), and mozobil (Plerixafor) up to two weeks prior to or concurrently with isolation of cells from the subject. Mobilized precursor cells can be obtained from the subject by any method known in the art, including, for example, leukapheresis 1-14 days following administration of the conditioning regimen. In some embodiments, specific precursor cell populations are further isolated by

Vectors. CARs may be prepared using vectors. Aspects of the present disclosure relate to an isolated nucleic acid sequence encoding a LHR, B7-H4, HLA-G, or HLA-DR CAR and vectors comprising, or alternatively consisting essentially of, or yet further consisting of, an isolated nucleic acid sequence encoding the CAR and its complement and equivalents of each thereof.

The preparation of exemplary vectors and the generation of CAR expressing cells using said vectors is discussed in detail in the examples below. In summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes.

In some embodiments, the isolated nucleic acid sequence encodes for a CAR comprising, or alternatively consisting essentially of, or yet further consisting of an antigen binding domain of an anti-LHR, B7-H4, HLA-G, or HLA-DR antibody, a CD8 α hinge domain, a CD8 α transmembrane domain, a CD28 costimulatory signaling region and/or a 4-1BB costimulatory signaling region, and a CD3 zeta signaling domain. In specific embodiments, the isolated nucleic acid sequence comprises, or alternatively consisting essentially thereof, or yet further consisting of, sequences encoding (a) an antigen binding domain of an anti-LHR, B7-H4, HLA-G, or HLA-DR antibody followed by (b) a CD8 α hinge domain, (c) a CD8 α transmembrane domain followed by (d) a CD28 costimulatory signaling region and/or a 4-1BB costimulatory signaling region followed by (e) a CD3 zeta signaling domain.

In some embodiments, the isolated nucleic acid sequence comprises, or alternatively consists essentially thereof, or yet further consists of, a Kozak consensus sequence upstream of the sequence encoding the antigen binding domain of the anti-LHR, B7-H4, HLA-G, or HLA-DR antibody. In some embodiments, the isolated nucleic acid comprises a polynucleotide conferring antibiotic resistance.

In some embodiments, the isolated nucleic acid sequence is comprised in a vector. In certain embodiments, the vector is a plasmid. In other embodiments, the vector is a viral vector. In specific embodiments, the vector is a lentiviral vector.

The preparation of exemplary vectors and the generation of CAR expressing cells using said vectors is discussed in detail in the examples below. In summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

In one aspect, the term “vector” intends a recombinant vector that retains the ability to infect and transduce non-dividing and/or slowly-dividing cells and integrate into the target cell's genome. In several aspects, the vector is derived from or based on a wild-type virus. In further aspects, the vector is derived from or based on a wild-type lentivirus. Examples of such, include without limitation, human immunodeficiency virus (HIV), equine infectious anemia virus (EIAV), simian immunodeficiency virus (SIV) and feline immunodeficiency virus (Hy). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses, and may also comprise synthetic components. Vector components can be manipulated to obtain desired characteristics, such as target cell specificity.

The recombinant vectors of this disclosure may be derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,07,993; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome. and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Packaging vector and cell lines. CARs can be packaged into a lentiviral or retroviral packaging system by using a packaging vector and cell lines. The packaging plasmid includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging plasmids comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging plasmid may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging plasmids and retroviral vectors expressing the LHR, B7-H4, HLA-G, or HLA-DR are transiently co-transfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the invention this transiently transfected first population of cells is then co-cultivated with mammalian target cells, for example human lymphocytes, to transduce the target cells with the foreign gene at high efficiencies. In yet another method of the invention the supernatants from the above described transiently transfected first population of cells are incubated with mammalian target cells, for example human lymphocytes or hematopoietic stem cells, to transduce the target cells with the foreign gene at high efficiencies.

In another aspect, the packaging plasmids are stably expressed in a first population of mammalian cells that are capable of producing virus, such as human embryonic kidney cells, for example 293 cells. Retroviral or lentiviral vectors are introduced into cells by either cotransfection with a selectable marker or infection with pseudotyped virus. In both cases, the vectors integrate. Alternatively, vectors can be introduced in an episomally maintained plasmid. High titer recombinant retrovirus-containing supernatants are produced.

Activation and Expansion of T Cells. Whether prior to or after genetic modification of the T cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041. Stimulation with the LHR, B7-H4, HLA-G, or HLA-DR antigen ex vivo can activate and expand the selected CAR expressing cell subpopulation. Alternatively, the cells may be activated in vivo by interaction with LHR, B7-H4, HLA-G, or HLA-DR antigen.

Methods of activating relevant cells are well known in the art and can be readily adapted to the present application; an exemplary method is described in the examples below. Isolation methods for use in relation to this disclosure include, but are not limited to Life Technologies Dynabeads® system activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD³/α-CD28 Dynabeads® may be used to activate and expand a population of isolated T-cells

III. Methods of Use

Therapeutic Application. The CAR T-cells of the present disclosure may be used to treat tumors and cancers. The CAR-T cells of the present invention may be administered either alone or in combination with diluents, known anti-cancer therapeutics, and/or with other components such as cytokines or other cell populations that are immunostimulatory.

Accordingly, method aspects of the present disclosure relate to methods for inhibiting the growth of a tumor in a subject in need thereof and/or for treating a cancer patient in need thereof. In some embodiments, the tumor is a solid tumor or a B-cell lymphoma or leukemia. In some embodiments, the tumors/cancer is thyroid, breast, colon, chiro-carcinoma, ovarian or prostate tumors/cancer or a B-cell lymphoma or leukemia. In some embodiments, the tumor or cancer expresses or overexpresses LHR, B7-H4, HLA-G, or HLA-DR. In certain embodiments, these methods comprise, or alternatively consist essentially of, or yet further consist of, administering to the subject or patient an effective amount of the isolated cell. In further embodiments, this isolated cell comprises a LHR, B7-H4, HLA-G, or HLA-DR CAR. In still further embodiments, the isolated cell is a T-cell or an NK cell. In some embodiments, the isolated cell is autologous to the subject or patient being treated. In a further aspect, the tumor expresses LHR, B7-H4, HLA-G, or HLA-DR antigen and the subject has been selected for the therapy by a diagnostic, such as the one described herein.

The CAR cells as disclosed herein may be administered either alone or in combination with diluents, known anti-cancer therapeutics, and/or with other components such as cytokines or other cell populations that are immunostimulatory. They may be administered as a first line therapy, a second line therapy, a third line therapy, or further therapy. As such, the disclosed CARs may be combined with other therapies (e.g., chemotherapy, radiation, etc.). Non-limiting examples of additional therapies include chemotherapeutics or biologics. Appropriate treatment regimens will be determined by the treating physician or veterinarian.

In some embodiments, the disclosed CARs may be delivered or administered into a cavity formed by the resection of tumor tissue (i.e. intracavity delivery) or directly into a tumor prior to resection (i.e. intratumoral delivery). In some embodiments, the disclosed CARs may be administered intravenously, intrathecally, intraperitoneally, intramuscularly, subcutaneously, or by other suitable means of administration.

Pharmaceutical compositions of the present invention may be administered in a manner appropriate to the disease to be treated or prevented. The quantity and frequency of administration will be determined by such factors as the condition of the patient, and the type and severity of the patient's disease, although appropriate dosages may be determined by clinical trials.

IV. Carriers

Additional aspects of the invention relate to compositions comprising a carrier and one or more of the products—e.g., an isolated cell comprising a LHR, B7-H4, HLA-G, or HLA-DR CAR, an isolated nucleic acid, a vector, an isolated cell of any anti-LHR, B7-H4, HLA-G, or HLA-DR antibody or CAR cell, an anti-LHR, B7-H4, HLA-G, or HLA-DR—described in the embodiments disclosed herein.

Briefly, pharmaceutical compositions of the present invention including but not limited to any one of the claimed compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Administration of the cells or compositions can be effected in one dose, continuously or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the invention can be administered in combination with other treatments.

The cells and populations of cell are administered to the host using methods known in the art and described, for example, in PCT/US2011/064191. This administration of the cells or compositions of the invention can be done to generate an animal model of the desired disease, disorder, or condition for experimental and screening assays.

Additional aspects of the invention relate to compositions comprising a carrier and one or more of the products—e.g., an isolated cell comprising a LHR, B7-H4, HLA-G, or HLA-DR CAR, an isolated nucleic acid, a vector, an isolated cell of any anti-LHR, B7-H4, HLA-G, or HLA-DR antibody or CAR cell, an anti-LHR, B7-H4, HLA-G, or HLA-DR—described in the embodiments disclosed herein.

Briefly, pharmaceutical compositions of the present invention including but not limited to any one of the claimed compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Briefly, pharmaceutical compositions of the present invention including but not limited to any one of the claimed compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials.

The following examples are illustrative of procedures which can be used in various instances in carrying the disclosure into effect.

### EXAMPLES

### Example 1

### Generation of Mouse Anti-LHR Monoclonal Antibodies

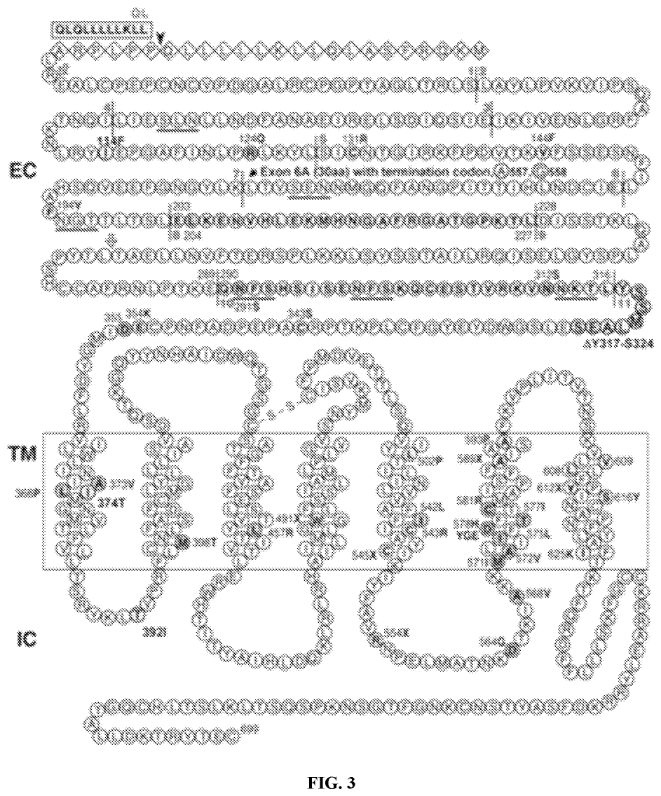

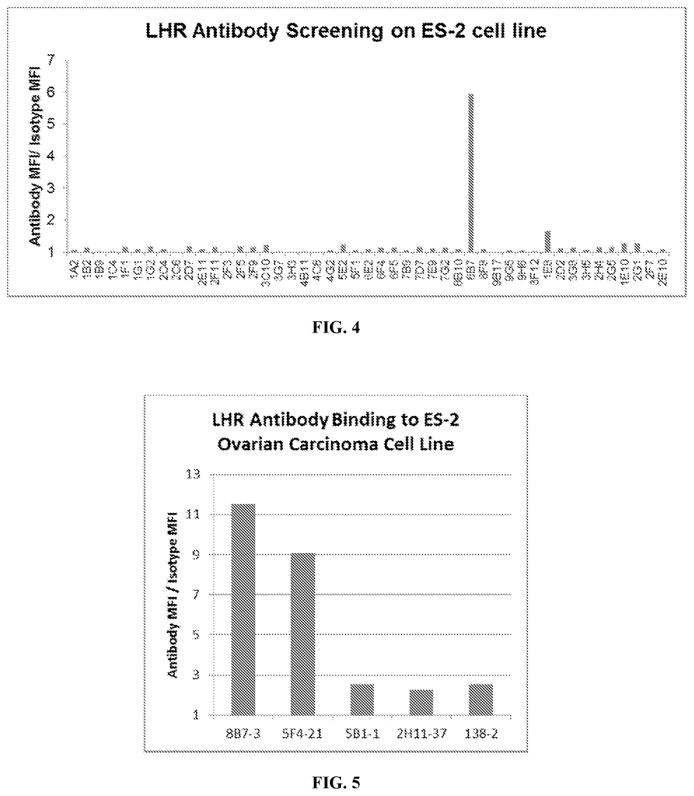

Antibodies against the lysine rich extracellular hormone binding domain of LHR were generated by repeated immunization of 4 week-old-BALB/c and NIH Swiss mice with genetically engineered LHR-Fc. As shown below in FIG. 3, the leader sequence and first part of the human LHR G-protein was used to generate the LHR-Fc used in the immunization and screening methods to generate and identify high binding antibodies. Since flow cytometry has previously been shown to be the best predictor of functional antibodies for CAR generation, this method was used to identify potential candidate antibodies from over 7 fusions performed in the laboratory. A typical flow cytometry screen of hybridomas positive by initial ELISA screen using LHR-Fc coated plates is shown below in FIG. 4 using the ES-2 ovarian carcinoma cell line. As seen in this figure with hybridoma 8B7, only rare LHR hybridomas were shown to produce high MFI by flow cytometry. These few candidate hybridomas were then subcloned by dilution in 96 well plates and expanded for freezing in vials. After further screening by flow cytometry, specific subclones were selected for large scale production using 2 L vessels (GRrex, 100 L, Wolfson). Filtered supernatants were then subjected to antibody purification using tandon protein A or G and ion exchange chromatography methods performed routinely in the laboratory. Once purified, five antibody subclones designated 8B7-3, 5F4-21, 5B1-1, 2H11-37, and 138-2 were sequenced to facilitate the engineering of single chain genes used for the construction of LHR CARs described below. For comparison, the 5 selected hybridoma subclones were tested on the ES-2 human ovarian carcinoma cell line by flow cytometry to demonstrated their relative mean fluorescence intensity (MFI) (FIG. 5).

### Example 2

### Anti-LHR Monoclonal Antibodies Detecting the Expression of LHR in Ovarian Cancer

The overall hypothesis is that ovarian cancer can be treated effectively and safely with LHR chimeric antigen receptor modified T-cells. As a target, LHR has significant advantages over other targets due to its common expression on ovarian cancers and its lack of expression on normal human tissues. LHR CAR T-cells are produced in vitro and in vivo to identify a potential clinical candidate for subsequent clinical trials or use with dual targeting CAR modified T-cells.

### Construction and Synthesis Single Chain LHR Antibody Genes

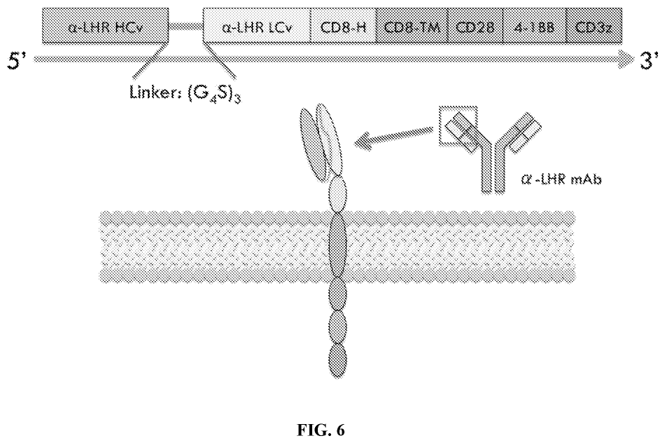

The DNA sequences for the 5 high binding anti-LHR antibodies (8B7-3, 5F4-21, 5B1-1, 2H11-37, and 138-2) were sequenced by MCLAB (South San Francisco, Calif.). All five antibodies are tested to determine which one produces the most effective CAR in assays described below. As shown below in FIG. 6, third generation CAR vectors were constructed consisting of the following tandem genes: a kozak consensus sequence; the CD8 signal peptide; the anti-LHR heavy chain variable region; a (Glycine4Serine)₃ flexible polypeptide linker; the respective anti-LHR light chain variable region; CD8 hinge and transmembrane domains; and the CD28, 4-1BB, and CD3ζ intracellular co-stimulatory signaling domains. Hinge, transmembrane, and signaling domain DNA sequences were known in the art (see US Patent Application No. 20130287748 A1). Anti-LHR CAR genes can be synthesized within a pUC57 vector backbone containing a beta-lactamase (“bla”) gene, which confers ampicillin resistance to the vector host. The pUC57 vector sequence is disclosed herein by referring to GeneBank accession No. Y14837 with the sequence of the beta-lactamase gene disclosed in the listed GeneBank accession No. The sequence associated with the listed GeneBank Accession number is herein incorporated by reference.

Subcloning of CAR Genes into Lentiviral Plasmids

NovaBlue Singles™ chemically-competent E. coli cells were transformed with anti-LHR plasmid cDNA. Following growth of the transformed E. coli cells, the CAR plasmids were purified and digested with the appropriate restriction enzymes inserted into an HIV-1-based lentiviral vector containing HIV-1 long terminal repeats (LTRs), packaging signal (Ψ), EF1α promoter, internal ribosome entry site (IRES), and woodchuck hepatitis virus post-transcriptional regulatory element (WPRE) via overnight T₄ DNA ligase reaction (New England Biosciences; Ipswich, Mass.). NovaBlue Singles™ chemically-competent E. coli cells are then transformed with the resulting anti-LHR containing lentiviral plasmid.

### Production of Lentiviral Particles

Prior to transfection, HEK293T cells were seeded at 4.0×10⁶ cells/100 mm tissue-culture-treated plate in 10 mL complete-Tet-DMEM and incubated overnight at 37° C. in a humidified 5% CO₂ incubator. Once 80-90% confluent, HEK293T cells were co-transfected with CAR-gene lentiviral plasmids and lentiviral packaging plasmids containing genes necessary to form lentiviral envelope and capsid components to facilitate the formation of plasmid-containing nanoparticles that bind HEK293T cells. After incubating transfected-HEK293T cell cultures for 4 hours at 37° C., the transfection medium was replaced with 10 mL fresh complete Tet DMEM. HEK293T cells are then incubated for an additional 48 hours, after which cell supernatants are harvested and tested for lentiviral particles via sandwich ELISA against p24, the main lentiviral capsid protein. Lentivirus-containing supernatants were aliquoted and stored at −80° C. until use for transduction of target CD4⁺ and CD8⁺ T cells.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) were recovered and washed by centrifugation with PBS containing 0.5% bovine serum albumin (BSA) and 2 mM EDTA. MACS CD4⁺ and CD8⁺ MicroBeads (Miltenyi Biotec; San Diego, Calif.) kits can be used to isolate these human T-cell subsets using magnetically activated LS columns to positive select for CD4⁺ and CD8⁺ T-cells. Magnetically-bound T-cells were then removed from the magnetic MACS separator, flushed from the LS column, and washed in fresh complete medium. The purity of CD4⁺ and CD8⁺ T-cell populations were assessed by flow cytometry using Life Technologies Acoustic Attune® Cytometer, and were enriched by Fluorescence-Activated Cell Sorting performed at USC's flow cytometry core facilities if needed. CD4⁺ and CD8⁺ T-cells were maintained at a density of 1.0×10⁶ cells/mL in complete medium supplemented with 100 IU/mL IL-2 in a suitable cell culture vessel, to which α-CD³/α-CD28 Human T-cell Dynabeads (Life Technologies; Carlsbad, Calif.) were added to activate cultured T cells. T-cells were incubated at 37° C. in a 5% CO₂ incubator for 2 days prior to transduction with CAR-lentiviral particles.

Lentiviral Transduction of CD4⁺ CD8⁺ T-Cells

Activated T-cells are collected and dead cells were removed by Ficoll-Hypaque density gradient centrifugation or the use of MACS Dead Cell Removal Kit (Miltenyi Biotec; San Diego, Calif.). In a 6-well plate, activated T-cells were plated at a concentration of 1.0×10⁶ cells/mL complete medium. To various wells, LHR CAR-containing lentiviral particles were added to cell suspensions at varying multiplicity of infections (MOIs), such as 1, 5, 10, and 50. Polybrene, a cationic polymer that aids transduction by facilitating interaction between lentiviral particles and the target cell surface, was added at a final concentration of 4 μg/mL. Plates are centrifuged at 800×g for 1 hour at 32° C. Following centrifugation, lentivirus-containing medium was aspirated and cell pellets are re-suspended in fresh complete medium with 100 IU/mL IL-2. Cells were placed in a 5% CO₂ humidified incubator at 37° C. overnight. Three days post-transduction, cells were pelleted and re-suspended in fresh complete medium with IL-2 and 400 μg/mL Geneticin (G418 sulfate) (Life Technologies; Carlsbad, Calif.). LHR CAR modified T-cells are assessed by flow cytometry and southern blot analysis to demonstrate successful transduction procedures. Prior to in vitro and in vivo assays, LHR CAR T-cells were enriched by FACS and mixed 1:1 for the in vivo studies.

### In Vitro Assessment of CAR Efficacy by Calcein-Release Cytotoxicity Assays

LHR antigen positive and negative target cells were collected, washed, and re-suspended in complete medium at a concentration of 1.0×10⁶ cells/mL. Calcein-acetoxymethyl (AM) was added to target cell samples at 15 which was then incubated at 37° C. in a 5% CO₂ humidified incubator for 30 minutes. Dyed positive and negative target cells were washed twice and re-suspended in complete medium by centrifugation and added to a 96-well plate at 1.0×10⁴ cells/well. LHR CAR T-cells was added to the plate in complete medium at effector-to-target cell ratios of 50:1, 5:1, and 1:1. Dyed-target cells suspended in complete medium and complete medium with 2% triton X-100 serves as spontaneous and maximal release controls, respectively. The plates were centrifuged at 365×g and 20° C. for 2 minutes before being placed back in the incubator for 3 hours. The plates were then centrifuged for 10 minutes and cell supernatants were aliquoted to respective wells on a black polystyrene 96-well plate and assessed for fluorescence on a Bio-Tek® Synergy™ HT microplate reader at excitation and emissions of 485/20 nm and 528/20 nm, respectively.

### Quantification of Human Cytokines by Luminex Bioassay.

Supernatants of LHR CAR modified T-cells and LHR positive and negative tumor cell lines were measured for cytokine secretion as a measure of CAR T-cell activation using standard procedures known in the art. Data were compared to medium alone and to cultures using unactivated human T-cells to identify background activity. The concentration of IL-2, IFN-g, IL-12, and other pertinent cytokines are measured over time during the incubation process.

### In Vivo Assessment of CAR T-Cell Efficacy in Two Xenograft Ovarian Cancer Models

LHR CAR T-cells are further evaluated in vivo using two different human ovarian cell xenograft tumor models. In the first model, solid human ovarian tumors are established subcutaneously in nude mice by injection of 5×10⁶ LHR positive ovarian cancer cell lines or LHR negative solid tumor cell lines. When the tumors reach 0.5 cm in diameter, groups of mice (n=5) are treated intravenously with 1 or 3×10⁷ human T-cells as negative controls or LHR CARs constructed from the most active LHR antibodies based upon the in vitro study results. Tumor volumes are then be measured by caliper 3×/week and volume growth curves are generated to demonstrate the effectiveness of experimental treatments over controls. In the second tumor model which is modified from Chekmasova et al. (Chekmasova, A. A. et al. (2010) Clin. Cancer Res. 16:3594-606), groups (n=5) of NOD/SCID/γ-chain −/−6-8 week old female mice (Jackson Laboratories, Inc.) are injected intraperitoneally with 3×10⁶ GFP transfected tumor cells from LHR positive or negative (control) human cell lines. Unlike Chekmasova et al. (Chekmasova, A. A. et al. (2010) Clin. Cancer Res. 16:3594-606) who treated mice 7 days after implantation, however, CAR T-cell therapy is not be initiated until the establishment of ascites at 3 weeks after implantation. At this time, 1 or 3×10⁷ LHR CAR T-cell preparations are injected intraperitoneally and.tumor volume is then monitored by fluorescent imaging weekly thereafter. Mice showing tumor progression are sacrificed at the appropriate time to alleviate morbidity. Kaplan Meier plots of mouse survival are generated from the data in order to compare the survival of control and experimental treatment groups. At sacrifice, blood and ascites are analyzed for the presence of CAR T-cells using human specific antibodies and flow cytometry. In addition, cytokine secretion is quantified by Luminex bead assay (Life Technologies, Inc.) for type 1 and 2 cytokines as a measure of CAR T-cell activation.

Studies with Dual Expressing CAR Modified T-Cells

In order to increase the specificity of LHR CAR modified T-cells, dual LHR CAR T-cells with either MUC-CD or mesothelin single chains are prepared. The principal of dual targeting CAR T-cells has successfully been tested in breast cancer using ERB/2 and MUC1 (Wilkie, S. et al. (2012) J. Clin. Immunol. 32:1059-1070), mesothelin and α-folate receptor (Lanitis, E. et al. (2013) Cancer Immunol. Res. 1:45-53), and PSMA and PSCA for the treatment of prostate cancer (Kloss, C. C. et al. (2013) Nat. Biotechnol. 31:71-75). MUC16, a mucin family member is over expressed on most ovarian cancers and is an established surrogate serum marker (CA125) for the progression and detection of ovarian cancers. MUC16 is composed of CA125, a large domain that gets cleaved, and a retained domain (MUC-CD) which contains an extracellular fragment, a transmembrane domain and cytoplasmic tail (Rao, T. D. et al. (2010) Appl. Immunohistochem. Mol. Morphology 18:462-472). MUC16 is also expressed at low levels in the uterus, endometrium, fallopian, tubes, ovaries and serosa of the abdominal and thoracic cavities. CAR modified MUC-CD targeted T cells exhibited efficient MUC-CD specific cytolytic activity against human ovarian cancer cell lines in vitro as well as successful eradication of established peritoneal ovarian tumors in SCID-Beige mice (Chekmasova, A. A. et al. (2010) Clin. Cancer Res. 16:3594-606). Hence, MUC-CD is a viable target for CAR therapy and an excellent choice for dual targeting CAR modified T-cells to reduce the potential on-target off-tumor effects. Both MUC-CD and mesothelin CAR modified T-cells have been shown to be effective, and in combination with LHR, may provide a safer alternative if required for optimal clinical use.

### Data and Statistical Analysis Plan

For the in vitro calcein-release assays, the percent of target cells lysed are compared using a one-way ANOVA, followed by an appropriate multiple comparisons test if significance (p<0.05) is found in the one-way ANOVA. In order to compare survival between CAR T-cells used in experimental and control groups in the ascites xenograft model, Kaplan Meier plots are constructed and a log rank test used to test for significance (p<0.05). For the subcutaneous tumor model, an ANOVA is used to compare tumor volume curves, followed by an appropriate multiple comparison test if significance (p<0.05) is found in the ANOVA.

TABLE 1
Expression of Three Potential Cell Surface
Targets (LHR, mesothelin, MUC16) on Nine Human
Ovarian Cell Lines Using Flow Cytometry.

| Ovarian Cell | | | |
|---|---|---|---|
| Line | LHR | Mesothelin | MUC16 |
| EFO-27 | + | − | − |
| EFO-21 | + | + | + |
| ES-2 | + | − | − |
| HEY | + | + | − |
| SKOV3 | + | + | − |
| TOV21G | + | + | − |
| NIHOVCAR3 | + | − | + |
| CAOV3 | + | − | + |
| SW626 | + | − | − |

TABLE 2
Immunohistochemical expression of LHR, MUC16, and Mesothelin on Panel
of Human Ovarian Tumors and Tissue Microarrays.

| Position | Pathology Diagnosis | Grade | Stage | Type | LHR | MUC16 | Mesothelin |
|---|---|---|---|---|---|---|---|
| A1 | Serous papillary | 1 | Ic | Malignant | + | + | + |
| | adenocarcinoma | | | | | | |
| A2 | Serous papillary | 2 | I | Malignant | + | + | + |
| | adenocarcinoma | | | | | | |
| A3 | Serous papillary | 2 | Ib | Malignant | + | − | − |
| | adenocarcinoma | | | | | | |
| A4 | Mucinous | 1 | Ia | Malignant | + | − | − |
| | adenocarcinoma | | | | | | |
| A5 | Serous papillary | 1 | Ic | Malignant | + | + | + |
| | adenocarcinoma with | | | | | | |
| | necrosis | | | | | | |
| A6 | Serous papillary | 2 | I | Malignant | + | + | + |
| | adenocarcinoma | | | | | | |
| A7 | Serous papillary | 2 | Ib | Malignant | + | − | − |
| | adenocarcinoma | | | | | | |
| A8 | Mucinous | 1 | Ia | Malignant | + | − | − |
| | adenocarcinoma | | | | | | |
| B1 | Mucinous | 1-2 | Ib | Malignant | + | − | − |
| | adenocarcinoma | | | | | | |
| B2 | Clear cell carcinoma | — | I | Malignant | + | + | − |
| B3 | Clear cell carcinoma | — | Ia | Malignant | + | − | − |
| B4 | Endometrioid | 1-2 | Ib | Malignant | + | − | − |
| | adenocarcinoma | | | | | | |
| B5 | Mucinous | 1-2 | Ib | Malignant | + | − | − |
| | adenocarcinoma | | | | | | |
| B6 | Clear cell carcinoma | — | I | Malignant | + | + | − |
| B7 | Clear cell carcinoma | — | Ia | Malignant | + | − | − |
| B8 | Endometrioid | 1-2 | Ib | Malignant | + | − | − |
| | adenocarcinoma with | | | | | | |
| | necrosis | | | | | | |
| C1 | Endometrioid | 2 | IIIc | Malignant | + | + | + |
| | adenocarcinoma | | | | | | |
| C2 | Granular cell tumor | — | I | Malignant | − | − | − |
| C3 | Ovary tissue | — | — | Normal | − | − | − |
| C4 | Ovary tissue | — | — | Normal | + | − | − |
| C5 | Endometrioid | 2 | IIIc | Malignant | + | + | + |
| | adenocarcinoma | | | | | | |
| C6 | Granular cell tumor | — | I | Malignant | − | − | − |
| C8 | Ovary tissue | — | — | Normal | − | − | − |
| Adrenal | Pheochromocytoma | — | — | Malignant | − | − | − |
| glad | (tissue marker) | | | | | | |

TABLE 3
Normal tissue reactivity of LHR, Mesothelin, and MUC-16 by
Immunohistochemistry

| | | Pathology | | | | |
|---|---|---|---|---|---|---|
| Position | Organ | Diagnosis | Type | LHR | MUC16 | Mesothelin |
| A1 | Cerebellum | Normal | Normal | − | − | − |
| A2 | Small | Normal | Normal | − | − | − |
| | intestine | | | | | |
| A3 | Lung | Normal | Normal | − | − | − |
| A4 | Pituitary | Normal | Normal | − | − | − |
| | Gland | | | | | |
| A5 | Spleen | Normal | Normal | − | − | − |
| A6 | Umbilical | Normal | Normal | − | − | − |
| | cord | | | | | |
| B1 | Cerebral | Normal | Normal | − | | − |
| | cortex | | | | | |
| B2 | Stomach | Normal | Normal | − | − | − |
| B3 | Liver | Cirrhosis | Normal | − | − | − |
| B4 | Parathyroid | Adenoma | Normal | − | − | − |
| B5 | Spinal cord | Normal | Normal | − | − | − |
| B6 | Thyroid | Normal | Normal | − | − | − |
| B7 | Tonsil | Reactive | Inflammation | − | − | − |
| C1 | Bone marrow | Normal | Normal | − | − | − |
| C2 | Fallopian | Normal | Normal | − | − | − |
| | tube | | | | | |
| C3 | Kidney | Normal | Normal | +? | +? | +? tubules |
| | | | | tubules | tubules? | |
| C4 | Pancreas | Normal | Normal | − | − | − |
| C5 | Skin | Normal | Normal | − | − | − |
| C6 | Thymus | Normal | Normal | − | − | − |
| C7 | Uterus | Normal | Normal | − | + | − |
| | | endometrium | | | | |
| D1 | Bladder | Normal epithelial | Normal | − | − | − |
| D2 | Breast | Normal | Normal | − | − | − |
| D3 | Heart | Normal | Normal | − | − | − |
| D4 | Ovary | Normal | Normal | − | − | − |
| D5 | Prostate | Normal | Normal | − | − | − |
| D6 | Testis | Normal | Normal | − | − | − |
| D7 | Uterus | Normal cervix | Normal | + | + | − |
| E1 | Adrenal | Normal | Normal | − | − | − |
| | Gland | | | | | |
| E2 | Breast | Normal | Normal | − | − | − |
| E3 | Colon | Normal | Normal | − | − | − |
| E4 | Lymph node | Reactive | Inflammation | − | − | − |
| E5 | Placenta | Normal | Normal | + | − | − |
| E6 | Stratified | Normal | Normal | − | − | − |
| | muscle | | | | | |
| E7 | Urethra | Normal | Normal | +/− | − | − |

TABLE 4

| Anti- | Heavy chain variable region | | |
|---|---|---|---|
| body | CDR1 | CDR2 | CDR3 |
| 5F4 | GYSITSGYG | IHYSGST | ARSLRY |
| 4A7 | GFSLTTYG | IWGDGST | AEGSSLFAY |
| 8B7 | GYSFTGYY | IYPYNGVS | ARERGLYQLRAMDY |
| Anti- | Light chain variable region | | |
| body | CDR1 | CDR2 | CDR3 |
| 5F4 | SSVNY | DTS | HQWSSYPYT |
| 4A7 | QSLLNSGNQKNY | WAS | QNDYSYPLT |
| 8B7 | QSISNN | NAS | QQSNSWPYT |

TABLE 5
LHR Staining in Normal Tissues

| Site | total cases | staining (positive/total) |
|---|---|---|
| Testis | 3 | 3/3 |
| Ovary | 3 | 3/3 |
| Appendix | 3 | 0/3 |
| Aorta | 3 | 0/3 |
| Bladder | 3 | 0/3 |
| Brain | 1 | 0/1 |
| Esophagus | 3 | 0/3 |
| Gallbladder | 3 | 0/3 |
| Heart | 3 | 0/3 |
| Kidney | 3 | 0/3 |
| Large intestine | 3 | 0/3 |
| Liver | 3 | 0/3 |
| Lung | 3 | 0/3 |
| Rectum | 3 | 0/3 |
| Small intestine | 3 | 0/3 |
| Spleen | 3 | 0/3 |
| Thyroid | 3 | 3/3 |
| Urethra | 3 | 0/3 |
| Vena cava | 3 | 0/3 |

### Example 3

### Anti-LHR CAR T-Cells

### Construction of the CAR Lentiviral Constructs

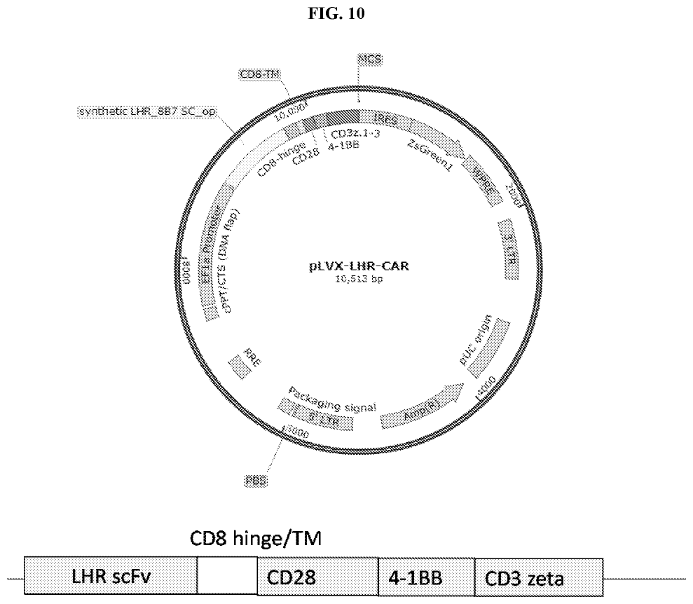

The CAR consists of an extracellular antigen binding moiety or scFV which binds LHR. The scFV is connected via a CD8 hinge region to the cytoplasmic signaling domain, comprised of the CD8 transmembrane region, and the signaling domains from CD28, 4-1BB and CD3z. The entire CAR sequence including the signaling domains, were synthetically synthesized by Genewiz Gene Synthesis Services (Piscataway, N.J.) (FIG. 10). The plasmids are purified and digested with the appropriate restriction enzymes to be inserted into an HIV-1-based, bicistronic lentiviral vector (pLVX-IRES-ZsGreen, Clontech, Signal Hill, Calif.) containing HIV-1 5′ and 3′ long terminal repeats (LTRs), packaging signal (Ψ), EF1α promoter, internal ribosome entry site (IRES), woodchuck hepatitis virus post-transcriptional regulatory element (WPRE), and simian virus 40 origin (SV40) via overnight T₄ DNA ligase reaction (New England Biosciences; Ipswich, Mass.). NovaBlue Singles™ chemically-competent E. coli cells are then transformed with the resulting LHR-CAR-containing lentiviral plasmid.

### Production of Lentiviral Particles

Prior to transfection, HEK 293T cells are seeded at 4.0×10⁶ cells/150 cm² tissue-culture-treated flask in 20 mL DMEM supplemented with 10% dialyzed FCS and incubated overnight at 37° C. in a humidified 5% CO₂ incubator. Once 80-90% confluent, HEK 293T cells are incubated in 20 ml DMEM supplemented with 1-% dialyzed FCS without penicillin/streptamycin for two hours in a 37° C. humidified 5% CO₂ incubator. HEK293T cells are co-transfected with the specific pLVX-CAR plasmid and lentiviral packaging plasmids containing genes necessary to form the lentiviral envelope & capsid components. A proprietary reaction buffer and polymer to facilitate the formation of plasmid-containing nanoparticles that bind HEK 293T cells are also added. After incubating the transfected-HEK 293T cell cultures for 24 hours at 37° C., the transfection medium is replaced with 20 mL fresh complete DMEM. Lentivirus supernatants are collected every 24 hours for three days and the supernatants will be spun down at 1,250 rpm for 5 minutes at 4° C., followed by filter sterilization and centrifugation in an ultracentrifuge at 20,000 g for 2 hrs at 4° C. The concentrated lentivirus is re-suspended in PBS containing 7% trehalose and 1% BSA for long term storage. The lentivirus is aliquoted and stored at −80° C. until use for transduction of target CD4⁺ and CD8⁺ T cells. The cell supernatants harvested after 24 hours are tested for lentiviral particles via sandwich ELISA against p24, the main lentiviral capsid protein. Transfection efficiency as determined by the expression of the protein marker ZsGreen, was estimated between 20%-50%, by visualization under a fluorescent microscope.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. T-cell enrichment kits (Stem Cell Technologies) are used to magnetically isolate these human T-cell subsets using negative selection for CD4⁺ and CD8⁺ T-cells. The purity of CD4⁺ and CD8⁺ T-cell populations are assessed by flow cytometry using Life Technologies Acoustic Attune® Cytometer, and are enriched by Fluorescence-Activated Cell Sorting. CD4⁺ and CD8⁺ T-cells mixed 1:1 are maintained at a density of 1.0×10⁶ cells/mL in complete 50% Click's medium/50% RPMI-1640 medium supplemented with 100 IU/mL IL-2 in a suitable cell culture vessel, to which α-CD³/α-CD28 Human T-cell activator beads (Stem Cell Technologies) are added to activate cultured T cells. T-cells are incubated at 37° C. in a 5% CO₂ incubator for 2 days prior to transduction with CAR lentiviral particles.

Lentiviral Transduction of CD4⁺ CD8⁺ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL in complete medium. Cells are transduced with the lentiviral particles supplemented with Lentiblast, a transfection aid (Oz Biosciences, San Diego, Calif.) to the cells. Transduced cells are incubated for 24 hours at 37° C. in a humidified 5% CO₂ incubator. The cells are then pelleted by centrifugation and the media changed, followed by addition of the T-cell activator beads (Stem Cell Technologies, San Diego, Calif.).

RT-PCR for mRNA Expression

mRNA from transduced T-cells are isolated using the Nucleospin RNA kit (Clontech, Signal Hill, Calif.). RT-PCR is run using the OneTaq One Step RNA kit (New England Biolabs, Boston, Mass.), using the following primers, 5′ CGCCTGTGATATCTACATCTGGGC 3′ and 5′ ATCGGCAGCTACAGCCATCT 3′. Samples are run on a 1% agarose gel.

### Cell Cytotoxicity Assays

Cytotoxicity of the CAR T-cells are determined using the lactate dehydrogenase (LDH) cytotoxicity kit (Thermo Scientific, Carlsbad, Calif.). Activated T-cells are collected and 1×10⁶ cells are transduced with the appropriate CAR lentiviral construct as described above. Cells are activated used the T-cell activator beads (Stem Cell Technologies, San Diego, Calif.) for two days prior to cytotoxicity assays. The optimal number of target cells is determined as per the manufacturer's protocol. For the assays, the appropriate target cells are plated in triplicate in a 96 well plate for 24 hours at 37° C. in a 5% CO₂ incubator, followed by the addition of activated CAR T-cells in ratios of 20:1, 10:1, 5:1 and 1:1, and incubated for 24 hours at 37° C. in a 5% CO₂ incubator. Cells are then lysed at 37° C. for 45 mins and centrifuged at 1,250 rpm for 5 mins. The supernatant is transferred to a fresh 96 well plate, followed by the addition of the reaction mixture for 30 minutes. The reaction is the stopped using the stop solution and the plate read at 450nm with an absorbance correction at 650 nm.

### In Vivo Tumor Regression Assay

Foxn1 null mice are injected with SKOV3, an ovarian tumor cell line which expresses LHR. Two ×106 cells in 200 ul of phosphate buffered saline are injected into the left flank of the mice using a 0.2 mL inoculum. Naive T-cells are activated for 2 days using the αCD3/CD28 activator complex (Stem Cell Technologies, San Diego, Calif.). The activated T-cells are then transduced with the pLVX-LHR-CAR lentiviral particles as described above, and activated for 2 days. 2.5×106 of the activated T-cells expressing the LHR CAR are injected intravenously into the mice on day 7 after tumor inoculation. Tumor sizes are assessed twice a week using Vernier calipers and the volume calculated.

### Cytotoxicity for LHR CAR T-Cells

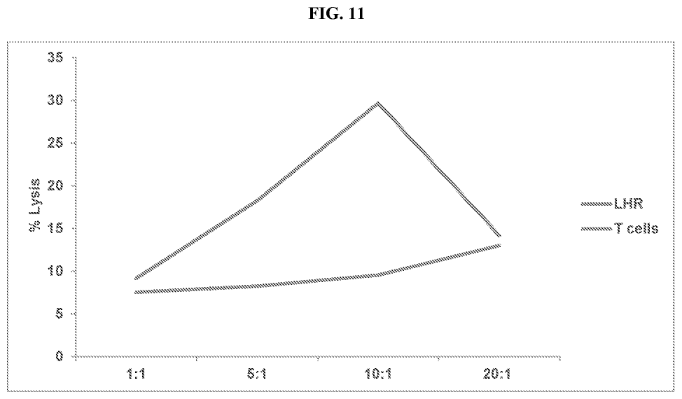

The cytolytic activity of the LHR CAR-T-cells was examined using the SKOV3 ovarian cancer cell line as target cells. SKOV3 was shown to express LHR by FACS analysis. CAR T-cells were added in ratios of 20:1, 10:1, 5:1 and 1:1 of effector cells to target cells. After 24 hours of incubation, the LHR CAR T-cells effectively lysed SKOV3 at a ratio of 10:1, showing a 30% lysis rate (FIG. 11). In comparison, uninduced T-cells did not show any cytotoxic activity at any of the ratios of effector cells to target cells used.

### RNA Expression for LHR CAR

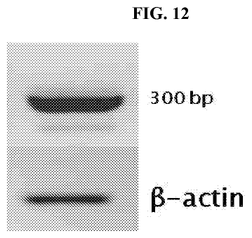

RT-PCR using mRNA isolated from T-cells transduced with the LHR CAR show mRNA expression of the chimeric CAR (FIG. 12). The RT-PCR was performed with primers that span the chimeric CAR between the CD8 hinge and the 4-1BB signaling domain, and is therefore highly specific to the expression of the CAR.

### Example 4

### Generation of Mouse Anti-Human B7-H4 Monoclonal Antibodies

### Construction of the B7H4-Fc Fusion Protein

Expression vector encoding the human B7-H4 signal and extracellular domains fused to the Fc region of human IgG₁ were constructed as follows: cDNA encoding the signal and extracellular domains of human B7-H4 were generated by PCR amplification from full-length cDNA purchased from Open Biosystem (Lafayette, Co.). The cDNA extends from the initiation Met in the signal sequence through Gly₂₃₆ of the total protein sequence. Primary PCR of B7-H4 was performed with the 5′ and 3′ primers 5′-TCG ATC AAG CTT GCC GCC ACC ATG GCT TCC CTG GGG CAG ATC-3′ AND 5′-TGT GTG AGT TTT GTC AGC CTT TGA CAG CTG-3′, respectively. The hinge-CH2-CH3 portion of human IgG₁ was PCR amplified with 5′ primer 5′-CTA AAC TCA AAG GCT GAC AAA ACT CAC ACA TGC CCA-3′ and 3′ primer 5′-TGA TTA ATG ATC AAT GAA TTC TCA TTT ACC CGG AGA CAG GGA-3′. The gene encoding huB7-H4-Fc was produced by assembling with 5′primer of B7-H4 and 3′ primer of human Fc, respectively. The full sequence of the B7-H4-Fc used was as follows (Bold: B7-H4); Non-bold: human Fc):

IGEDGILSCTFEPDIKLSDIVIQWLKEGVLGLVHEFKEGKDELSEQDEMF
RGRTAVFADQVIVGNASLRLKNVQLTDAGTYKCYIITSKGKGNANLEYKT
GAFSMPEVNVDYNASSETLRCEAPRWFPQPTVVIVASQVDQGANFSEVSN
TSFELNSENVTMKVVSVLYNVTINNTYSCMIENDIAKATGDIKVTESEIK
RRSHLQLLNSKADKTHTCPPCPAPELLGGPSVFLEPPKPKDTLMISRTPE
VTCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYS
KLTVDKSRWQQGNVFSCSVMHEALHNHYTQKSLSLSPGK

The B7H4-Fc fusion gene was then digested with Hind3 and EcoRI and inserted into Hind3 and EcoRI sites of pN24 expression vector, resulting in the expression vector pN24/B7-H4-Fc.

### Expression, Purification, and Characterization of B7-H4-Fc Antigen

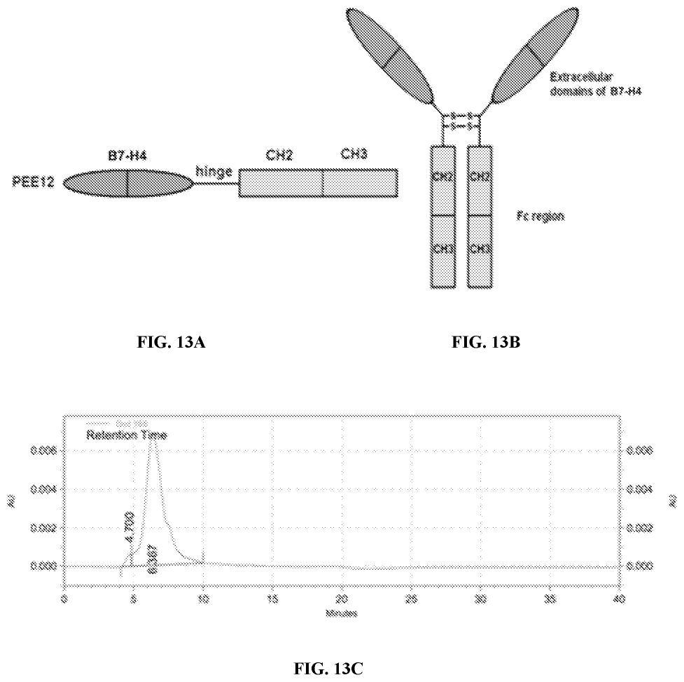

B7-H4-Fc fusion protein was expressed in NSO murine myeloma cells for long-term stable expression according to the manufacturer's protocol (Lonza Biologics, Inc.). The highest producing clone was scaled up for incubation in an aerated 3L stir-flask bioreactor using 3% heat-inactivated dialyzed fetal calf serum. The fusion protein was then purified from the filtered spent culture medium by sequential Protein A affinity chromatography and ion-exchange chromatography procedures. The fusion protein was analyzed by HPLC and sodium dodecyl sulfate-polyacrylamide gel electrophoresis (SDS-PAGE) under reducing conditions and stained with Coomassie Blue to demonstrate proper assembly and purity. A schematic of the completed vector and molecule is shown in FIGS. 13A-13C along with HPLC data verifying its size.

### Immunization Procedures

Four week old female BALB/c mice purchased from Harlan Laboratories were immunized every two weeks ×4 with 10 ug of KLH-conjugated huB7-H4-Fc emulsified with Complete Freund's Adjuvant (first and second immunization) or incomplete Freund's Adjuvant (third and fourth immunization). Mice were injected intradermally with a total of 25 ug of antigen/adjuvant divided into three separate spots on the back of the mice per immunization. Ten days after the last immunization, blood samples were obtained and tittered by ELISA procedures on antigen coated plates. Mice showing the highest titers then received a fifth immunization boost of B7-H4-Fc without adjuvant or KLH conjugation intravenously in which 10 ug were injected via the lateral tail vein in a 100 ul solution of sterile Phosphate Buffered Saline.

### Hybridoma Production

Four days later, boosted mice were sacrificed and the spleens removed for the hybridoma procedure. After dispersing the splenocytes in a solution of RPMI-1640 medium containing Pen/Strep antibiotics, the splenocytes were fused with murine NSO cells using PEG (Hybri MAX, mol wt 1450, Cat. No: p7181, Sigma). HAT selection was then used to enable only fused cells to grow. Supernatant from wells with growing hybridoma cells were then screened initially by ELISA against B7-H4-Fc antigen coated plates and secondarily by flow cytometry on B7-H4 positive and negative human tumor cell lines (SK-BR-3 and HT-29, respectively). To eliminate positive hybridomas to the Fc region of B7-H4-Fc, supernatants were also screened against IL-2-Fc coated plates and those clones showing positivity to both antigens, were eliminated from further study. Hybridomas showing a positive and high mean fluorescent index (MFI) were selected for subcloning by limiting dilution methods. Subclones were then retested by flow cytometry, frozen in liquid nitrogen, and expanded in 2 L vessels before antibody was purified by tandori Protein A or G and ion exchange chromatography methods. Purified antibodies were then vialed and stored at −20° C. until used.

### Flow Cytometry Data

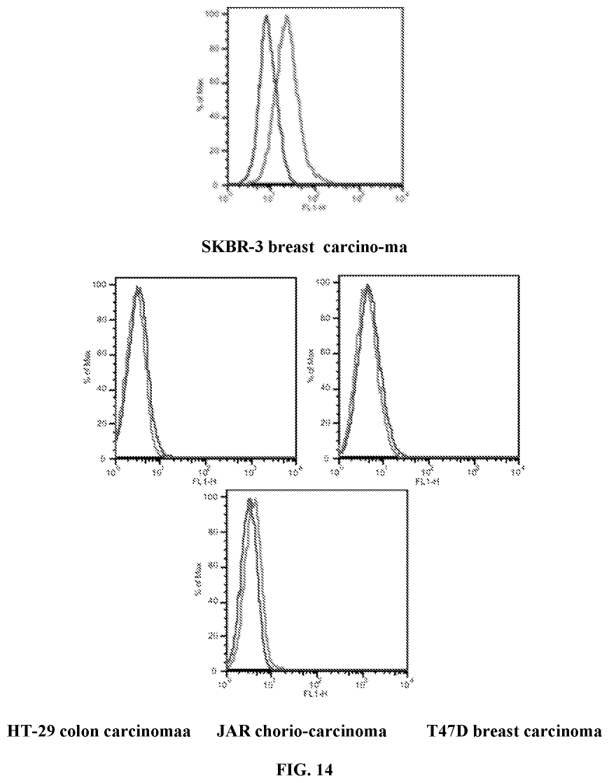

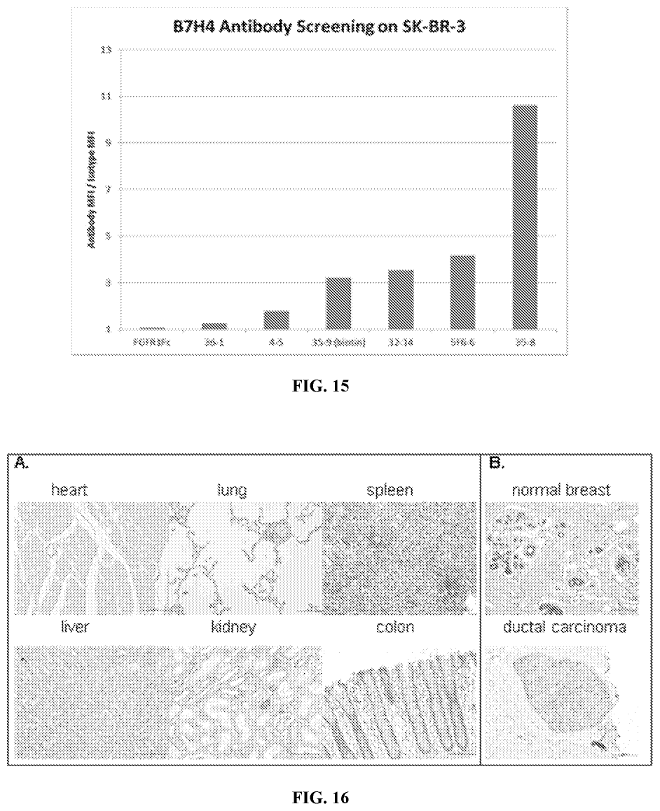

To determine the best binding antibodies, flow cytometry was performed on B7-H4 positive (SK-BR-3) and negative (HT-29, JAR, and T47D) cell lines using aliquots of purified antibodies. As shown in FIG. 14, positive cell lines had increased binding characteristics compared to negative antibody isotype controls. A comparison of positive subclones showed that hybridomas 35-8 and 5F6-6 produced the highest MFI to the B7-H4 expressing SK-BR-3 cell line (FIG. 15) and were therefore selected as candidates for CAR T-cell construction as described below.

### Immunohistochemistry Data

Using these monoclonal antibodies, tissue microarrays (FDA808c, Biomax, Inc.) of human normal tissues were screened to determine antibody binding in 24 organs, with 3 donors per organ. While most tissues were negative for staining, there was inconsistent cytoplasmic staining in epithelial cells of the gastrointestinal tract, and in the proximal and distal convoluted tubules of the kidneys (FIGS. 16A-16B). Strong, consistent membranous staining was only found in the apical portion of breast ductal cells and in some of the tubules in the kidney (FIGS. 16A-16B). Staining in normal breast tissue, however, paled in comparison to staining in breast cancer tissue as shown below, where strong membranous and cytoplasmic staining was noted in five out of five different cancer cases.

From the antibodies generated against human B7-H4-Fc, two monoclonal antibodies have been shown to produce high binding profiles by flow cytometry against B7-H4 positive but not negative tumor cells lines. To prevent the possibility of a human anti-mouse response against B7-H4 CAR T-cell, humanized antibodies can be generated for their construction prior to their use in patients.

### Example 5

### Generation of B7-H4 CAR T-Cells

### Construction and Synthesis of Single Chain Anti-Human B7-H4 Antibody Genes

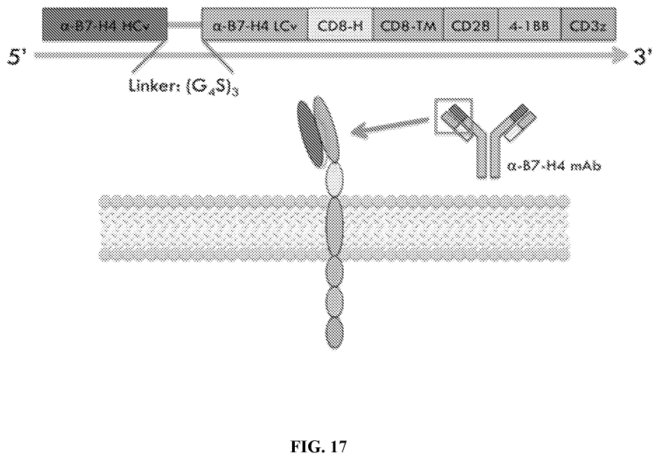

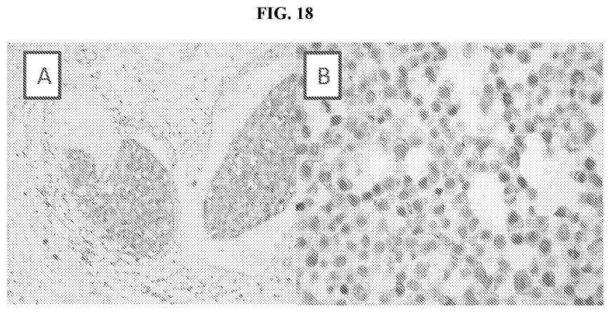

The DNA sequences for 35-8 and 5F6-6 high binding anti-B7-H4 antibodies generated are obtained from MCLAB (South San Francisco, Calif.). Both antibodies are tested to determine which one produces the most effective CAR T-cells in assays described below. For these studies, second or third (FIG. 17) generation CAR vectors are constructed consisting of the following tandem genes: a kozak consensus sequence; the CD8 signal peptide; the anti-B7-H4 heavy chain variable region; a (Glycine4Serine)3 flexible polypeptide linker; the respective anti-B7-H4 light chain variable region; CD8 hinge and transmembrane domains; and the CD28, 4-1BB, and CD3 intracellular co-stimulatory signaling domains. Hinge, transmembrane, and signaling domain DNA sequences are ascertained from a patent by Carl June (see US 20130287748 A1). Anti-B7-H4 CAR genes are synthesized by Genewiz, Inc. (South Plainfield, N.J.) within a pUC57 vector backbone containing the bla gene, which confers ampicillin resistance to the vector host.

Subcloning of CAR Genes into Lentiviral Plasmids

NovaBlue Singles™ chemically-competent E. coli cells are transformed with anti-B7-H4 plasmid cDNA. Following growth of the transformed E. coli cells, the CAR plasmids are purified and digested with the appropriate restriction enzymes to be inserted into an HIV-1-based lentiviral vector containing HIV-1 long terminal repeats (LTRs), packaging signal (Ψ), EF1α promoter, internal ribosome entry site (IRES), and woodchuck hepatitis virus post-transcriptional regulatory element (WPRE) via overnight T4 DNA ligase reaction (New England Biosciences; Ipswich, Mass.). NovaBlue Singles™ chemically-competent E. coli cells are then transformed with the resulting anti-B7-H4 containing lentiviral plasmid.

### Production of Lentiviral Particles

Prior to transfection, HEK293T cells are seeded at 4.0×10⁶ cells/100 mm tissue-culture-treated plate in 10 mL complete-Tet-DMEM and incubated overnight at 37° C. in a humidified 5% CO₂ incubator. Once 80-90% confluent, HEK293T cells are co-transfected with CAR-gene lentiviral plasmids and lentiviral packaging plasmids containing genes necessary to form lentiviral envelope & capsid components, in addition to a proprietary reaction buffer and polymer to facilitate the formation of plasmid-containing nanoparticles that bind HEK293T cells. After incubating transfected-HEK293T cell cultures for 4 hours at 37° C., the transfection medium is replaced with 10 mL fresh complete Tet DMEM. HEK293T cells are then incubated for an additional 48 hours, after which cell supernatants are be harvested and tested for lentiviral particles via sandwich ELISA against p24, the main lentiviral capsid protein. Lentivirus-containing supernatants are aliquoted and stored at −80° C. until use for transduction of target CD4⁺ and CD8⁺ T cells.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. MACS CD4⁺ and CD8⁺ MicroBeads (Miltenyi Biotec; San Diego, Calif.) kits are used to isolate these human T-cell subsets using magnetically activated LS columns to positive select for CD4⁺ and CD8⁺ T-cells. Magnetically-bound T-cells are then removed from the magnetic MACS separator, flushed from the LS column, and washed in fresh complete medium. The purity of CD4⁺ and CD8⁺ T-cell populations are assessed by flow cytometry using Life Technologies Acoustic Attune® Cytometer, and are enriched by Fluorescence-Activated Cell Sorting performed at USC's flow cytometry core facilities if needed. CD4⁺ and CD8⁺ T-cells are maintained at a density of 1.0×10⁶ cells/mL in complete medium supplemented with 100 IU/mL IL-2 in a suitable cell culture vessel, to which α-CD³/α-CD28 Human T-cell Dynabeads (Life Technologies; Carlsbad, Calif.) are added to activate cultured T cells. T-cells are incubated at 37° C. in a 5% CO₂ incubator for 2 days prior to transduction with CAR-lentiviral particles.

### Lentiviral Transduction of CD4+ CD8+ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL complete medium. To various wells, B7-H4 CAR-containing lentiviral particles are added to cell suspensions at varying multiplicity of infections (MOIs), such as 1, 5, 10, and 50. Polybrene, a cationic polymer that aids transduction by facilitating interaction between lentiviral particles and the target cell surface, is added at a final concentration of 4 μg/mL. Plates are centrifuged at 800×g for lhr at 32° C. Following centrifugation, lentivirus-containing medium is aspirated and cell pellets are resuspended in fresh complete medium with 100 IU/mL IL-2. Cells are placed in a 5% CO₂ humidified incubator at 37° C. overnight. Three days post-transduction, cells are pelleted and resuspended in fresh complete medium with IL-2 and 400 μg/mL Geneticin (G418 sulfate) (Life Technologies; Carlsbad, Calif.). B7-H4 CAR modified T-cells are assessed by flow cytometry and southern blot analysis to demonstrate successful transduction procedures. Prior to in vitro and in vivo assays, B7-H4 CAR T-cells are enriched by FACS and mixed 1:1 for the in vivo studies.

### In Vitro Assessment of CAR Efficacy by Calcein-Release Cytotoxicity Assays

B7-H4 antigen positive and negative human cell lines are collected, washed, and resuspended in complete medium at a concentration of 1.0×10⁶ cells/mL. Calcein-acetoxymethyl (AM) are added to target cell samples at 15 which are then incubated at 37° C. in a 5% CO₂ humidified incubator for 30 minutes. Dyed positive and negative target cells are washed twice and resuspended in complete medium by centrifugation and added to a 96-well plate at 1.0×10⁴ cells/well. B7-H4 CAR T-cells are added to the plate in complete medium at effector-to-target cell ratios of 50:1, 5:1, and 1:1. Dyed-target cells suspended in complete medium and complete medium with 2% triton X-100 serve as spontaneous and maximal release controls, respectively. The plates are centrifuged at 365×g and 20° C. for 2 minutes before being placed back in the incubator 3 hours. The plates are then centrifuged 10 minutes and cell supernatants are aliquoted to respective wells on a black polystyrene 96-well plate and assessed for fluorescence on a Bio-Tek® Synergy™ HT microplate reader at excitation and emissions of 485/20 nm and 528/20 nm, respectively.

### Quantification of Human Cytokines by Luminex Bioassay.

Supernatants of B7-H4 CAR modified T-cells and B7-H4 positive and negative tumor cell lines are measured for cytokine secretion as a measure of CAR T-cell activation using standard procedures performed routinely in the laboratory. Data are compared to medium alone and to cultures using non-activated human T-cells to identify background activity. The concentration of IL-2, IFN-g, IL-12, and other pertinent cytokines are measured over time during the incubation process.

### In Vivo Assessment of CAR T-Cell Efficacy in Two Xenograft B7-H4 Positive Cancer Models

B7-H4 CAR T-cells are further evaluated in vivo using two different human tumor cell line xenograft tumor models. For both, solid tumors are established subcutaneously in 6-8 week old female nude mice by injection of 5×106 B7-H4 positive or negative solid tumor cell lines. When the tumors reach 0.5 cm in diameter, groups of mice (n=5) are treated intravenously with 1 or 3×10⁷ human T-cells as negative controls or B7-H4 CAR T-cells constructed from the candidate B7-H4 antibodies based upon the in vitro study results. Tumor volumes are then measured by caliper 3×/week and volume growth curves are generated to demonstrate the effectiveness of experimental treatments over controls.

In general, B7-H4 is expressed on tumors to suppress the immune response. Its expression on normal tissues is very limited making it a viable target for CAR T-cells.

### Example 6

### Anti-B7-H4 CAR T-Cells

### Construction of the CAR Lentiviral Constructs

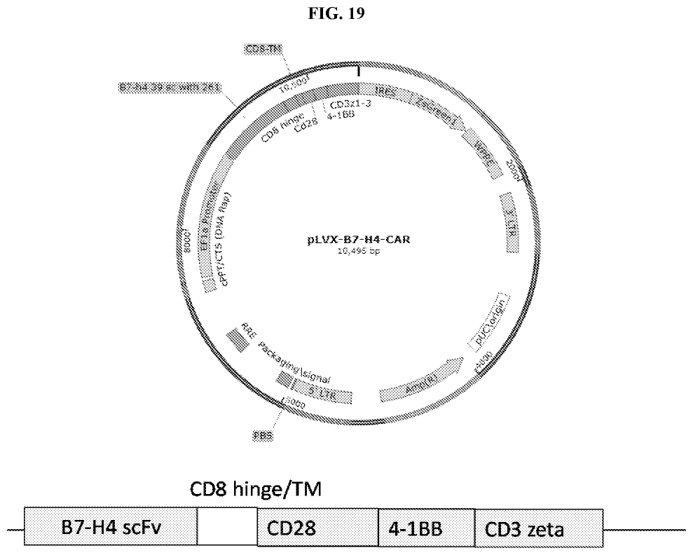

The CAR consists of an extracellular antigen binding moiety or scFV which binds B7-H4. The scFV is connected via a CD8 hinge region to the cytoplasmic signaling domain, comprised of the CD8 transmembrane region, and the signaling domains from CD28, 4-1BB and CD3z (FIG. 19). The scFV sequence including the signaling domains, were synthetically synthesized by Genewiz Gene Synthesis services (Piscataway, N.J.). The plasmids are purified and digested with the appropriate restriction enzymes to be inserted into an HIV-1-based bicistronic lentiviral vector (pLVX-IRES-ZsGreen, Clontech, Signal Hill, Calif.) containing HIV-1 5′ and 3′ long terminal repeats (LTRs), packaging signal (Ψ), EF 1α promoter, internal ribosome entry site (IRES), woodchuck hepatitis virus post-transcriptional regulatory element (WPRE) and simian virus 40 origin (SV40) via overnight T4 DNA ligase reaction (New England Biosciences; Ipswich, Mass.). NovaBlue Singles™ chemically-competent E. coli cells will then be transformed with the resulting CAR-containing lentiviral plasmid.

### Production of Lentiviral Particles

Prior to transfection, HEK 293T cells are seeded at 4.0×106 cells in a 150 cm2 tissue-culture-treated flask in 20 mL DMEM supplemented with 10% dialyzed FCS and incubated overnight at 37° C. in a humidified 5% CO2 incubator. Once 80-90% confluent, HEK 293T cells are incubated in 20 mL DMEM supplemented with 1-% dialyzed FCS without penicillin/streptamycin for two hours in at 37° C. in a humidified 5% CO2 incubator. HEK293T cells are co-transfected with the pLVX-B7-H4-CAR plasmid and lentiviral packaging plasmids containing genes necessary to form the lentiviral envelope & capsid components. A proprietary reaction buffer and polymer to facilitate the formation of plasmid-containing nanoparticles that bind HEK 293T cells are also added. After incubating the transfected-HEK 293T cell cultures for 24 hours at 37° C., the transfection medium is replaced with 20 mL fresh complete DMEM. Lentivirus supernatants is then collected every 24 hours for three days and the supernatants centrifuged at 1,250 rpm for 5 mins at 4° C., followed by filter sterilization and centrifugation in an ultracentrifuge at 20,000 g for 2 hrs at 4° C. The concentrated lentivirus is re-suspended in PBS containing 7% trehalose and 1% BSA. The lentivirus is then aliquoted and stored at −80° C. until use for transduction of target CD4+ and CD8+ T cells. The cell supernatants harvested after 24 hours are tested for lentiviral particles via sandwich ELISA against p24, the main lentiviral capsid protein. Transfection efficiency as determined by the expression of the protein marker ZsGreen was estimated between 20%-50%, by visualization under a fluorescent microscope.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. T-cell enrichment kits (Stem Cell Technologies) are used to isolate these human T-cell subsets magnetically using negative selection for CD4+ and CD8+ T-cells. The purity of CD4+ and CD8+ T-cell populations are assessed by flow cytometry using Life Technologies Acoustic Attune® Cytometer, and are enriched by Fluorescence-Activated Cell Sorting. CD4+ and CD8+ T-cells mixed 1:1 are maintained at a density of 1.0×106 cells/mL in complete 50% Click's medium/50 RPMI-1640 medium supplemented with 100 IU/mL IL-2 in a suitable cell culture vessel, to which αCD³/αCD28 Human T-cell activator beads (Stem Cell Technologies) are added to activate the cultured T cells. T-cells are then incubated at 37° C. in a 5% CO2 incubator for 2 days prior to transduction with CAR lentiviral particles.

Lentiviral Transduction of CD4⁺ CD8⁺ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×106 cells/mL in complete medium. Cells are then transduced with the lentiviral particles supplemented with Lentiblast, a transfection aid (Oz Biosciences, San Diego, Calif.) to the cells. Transduced cells are incubated for 24 hours at 37° C. in a humidified 5% CO2 incubator. The cells are spun down and the media changed, followed by addition of the T-cell activator beads (Stem Cell Technologies, San Diego, Calif.).

### Cell Cytotoxicity Assays

Cytotoxicity of the CAR T-cells are determined using the lactate dehydrogenase (LDH) cytotoxicity kit (Thermo Scientific, Carlsbad, Calif.). Activated T-cells are collected and 1×10⁶ cells are transduced with the B7-H4 CAR lentiviral construct as described above. Cells are activated used the T-cell activator beads (Stem Cell Technologies, San Diego, Calif.) for two days prior to cytotoxicity assays. The optimal number of target cells is determined as per manufacturer's protocol. For the assays, the appropriate target cells are plated in triplicate in a 96 well plate for 24 hours at 37° C. in a 5% CO₂ incubator, followed by addition of activated CAR T-cells in ratios of 20:1, 10:1, 5:1 and 1:1, and incubated for 24 hours at 37° C. in a 5% CO₂ incubator. Cells are then lysed at 37° C. for 45 mins and centrifuged at 1,250 rpm for 5 mins. The supernatant is transferred to a fresh 96 well plate, followed by the addition of the reaction mixture for 30 mins. The reaction will be stopped using the stop solution and the plate read at 450nm with an absorbance correction at 650 nm.

### In Vivo Tumor Regression Assay

Foxn1 null mice are injected with immortalized breast carcinoma cell line MDA-MB-468, which expresses B7-H4. Two ×106 tumor cells in 200 ul of phosphate buffered saline (PBS) are injected into the left flank of the mice using a 0.2 mL inoculum. T-cells are activated for 2 days with the αCD3/CD28 activator complex (Stem Cell Technologies, San Diego, Calif.). The activated T-cells are then transduced with B7-H4 CAR lentiviral particles, followed by activation with the αCD3/CD28 activator complex for an additional 2 days. The activated B7-H4 CAR T-cells (2.5×106) are then injected intravenously into the mice on day 7 after tumor inoculation. Tumor sizes are assessed twice a week using Vernier calipers and the volume calculated.

### Cytotoxicity for B7-H4 CAR T-Cells

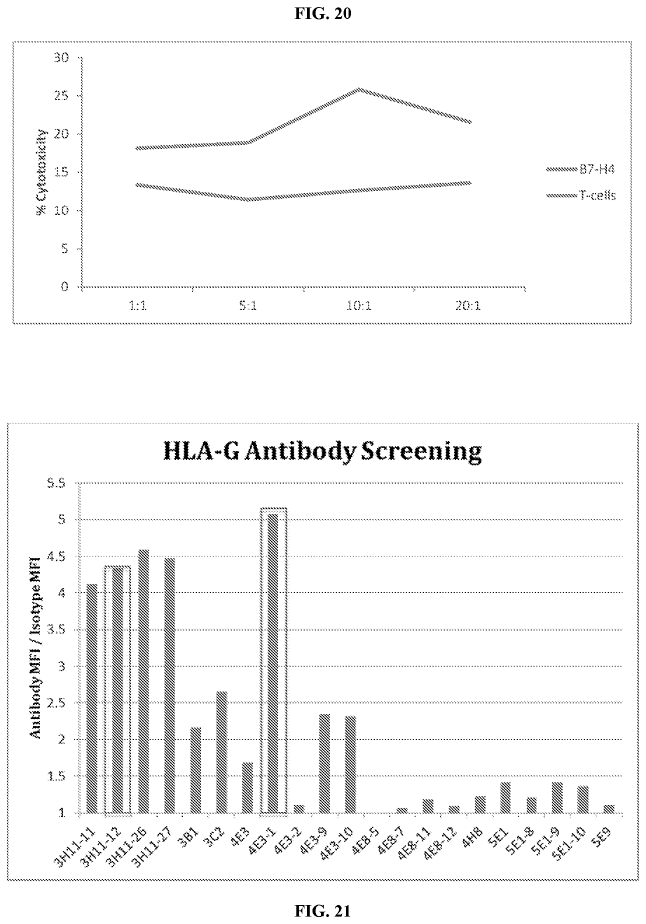

The cytolytic activity of the B7-H4 CAR T-cells was examined using SKBR3, a breast carcinoma cell line. SKBR3 expresses B7-H4, as determined by FACS analysis (FIG. 20). B7-H4 CAR T-cells were added to the SKBR3 in ratios of 20:1, 10:1, 5:1 and 1:1 of effector to target cells. At a ratio of 10,000:1, B7-H4 CAR T-cells show increased lysis of the target SKBR3 cells with a lysis rate of 25%. In comparison, untransduced T-cells did not lyse SKBR3 cells at any of the ratios tested.

### Example 7

### Generation of Mouse Anti-Human HLA-G Monoclonal Antibodies Antigen

The HLA Class I Histocompatibility Antigen, alpha chain G antigen was purchased from MybioSource.com (catalogue number MBS717410). It is a recombinant protein made in bacteria and has a HIS Tag, a molecular weight of 50 KD (90% purity), and a sequence of:

GSHSMRYFSA AVSRPGRGEP RFIAMGYVDD TQFVRFDSDS
ACPRMEPRAP WVEQEGPEYW EEETRNTKAH AQTDRMNLQT
LRGYYNQSEA SSHTLQWMIG CDLGSDGRLL RGYEQYAYDG
KDYLALNEDL RSWTAADTAA QISKRKCEAA NVAEQRRAYL
EGTCVEWHLA-G YLENGKEMLQ RADPPKTHVT HHPVFDYEAT
LRCWALGFYP AEIILTWQRD GEDQTQDVEL VETRPAGDGT
FQKWAAVVVP SGEEQRYTCH VQHEGLPEPL MLRWKQSSLP
TIPEVIGI VAGLVVLAAV VTGAAVAAVL WRKKSSD.

### Immunization Procedures

Four week old female BALB/c mice purchased from Harlan Laboratories were immunized every two weeks ×4 with 10 μg of antigen emulsified with Complete Freund's Adjuvant (first and second immunization) or incomplete Freund's Adjuvant (third and fourth immunization). Mice were injected intradermally with a total of 25 μg of antigen/adjuvant divided into three separate spots on the back of the mice per immunization. Ten days after the last immunization, blood samples were obtained and tittered by ELISA procedures on antigen coated plates. Mice showing the highest titers then received a fifth immunization boost intravenously without adjuvant in which 10 μg were injected via the lateral tail vein in a 100 μl solution of sterile Phosphate Buffered Saline.

### Generation of Hybridomas

Four days later, these mice were sacrificed and the spleens removed for the hybridoma procedure. After dispersing the splenocytes in a solution of RPMI-1640 medium containing Pen/Strep antibiotics, the splenocytes were fused with murine NSO cells using PEG (Hybri MAX, mol wt 1450, Cat. No: p′7181, Sigma). HAT selection was then used to enable only fused cells to grow. Supernatant from wells with growing hybridoma cells were then screened initially by ELISA against antigen coated plates and secondarily by flow cytometry on HLA-G positive and negative human tumor cell lines (JAR Trophoblastic Carcinoma). Hybridomas showing a positive and high mean fluorescent index (MFI) were selected for subcloning by limiting dilution methods. Subclones were then retested by flow cytometry, frozen in liquid nitrogen, and expanded in 2 L vessels to before antibody was purified by tandon Protein A or G and ion exchange chromatography methods. Purified antibodies were then vialed and stored at −20° C. until used.

### Flow Cytometry Procedures and Data

Screening methods using flow cytometry were performed on HLA-G positive (JEG-3 trhophoblastic carcinoma) and negative (K562, Jurkat) cell lines using supernatant from hybridomas found positive by ELISA to antigen coated plates. Those hybridomas producing high mean fluorescent indexes (MFI) were then subcloned and rescreened for selective positivity to HLA-G. As shown below in FIG. 21, subclones of parental hybridomas 3H11 and 4E3 continued to produce high MFI to the HLA-G expressing JEG-3 cell line. From these data, 3H11-12 and 4E3-1 were selected to generate CAR-T cells as described below.

Immunohistochemistry with Selected Antibodies

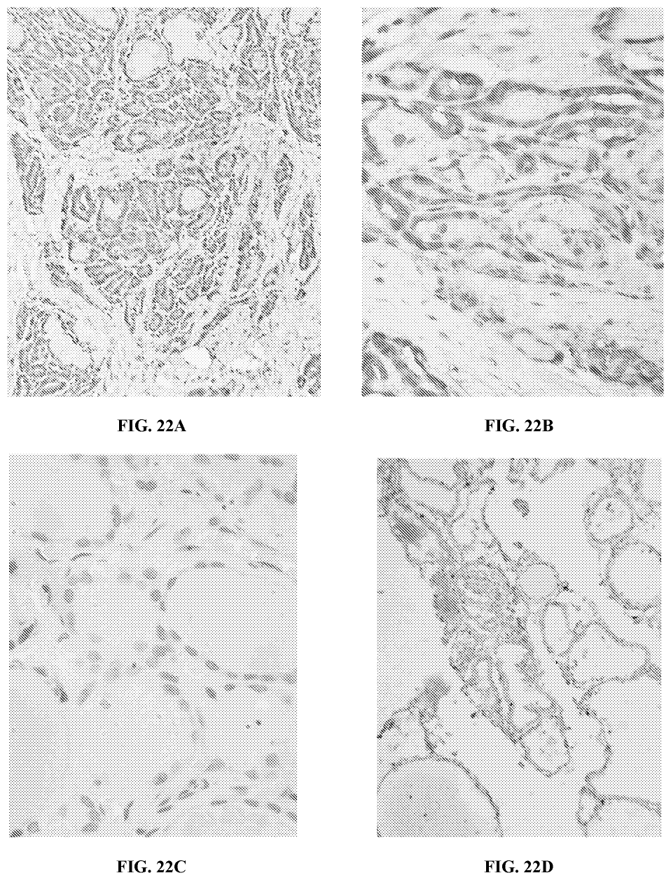

Antibody 4E3 and its subclones were found to stain HLA-G positive tissues using standard immunohistochemical procedures and antigen retrieval methods. As shown in FIGS. 22A-22D, HLA-G positivity was seen both in the cytoplasm and cell membrane of antigen positive tumors such as papillary thyroid carcinoma (FIGS. 22A, 22B) but was negative in normal thyroid tissues (FIG. 22C) which retained its HLA expression (FIG. 22D). The availability of a companion diagnostic antibody for HLA-G using immunohistochemistry will enable the identification of patients likely to benefit from HLA-G CAR T-cell therapy in upcoming clinical trials.

### Example 8

### Generation of HLA-G CAR T-Cells

### Construction and Synthesis Single Chain HLA-G Antibody Genes

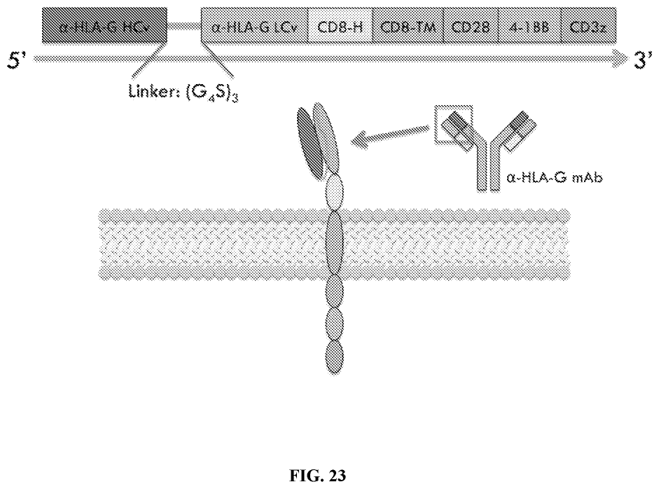

The DNA sequences for 2 high binding anti-HLA-G antibodies generated in our laboratory (4E3-1 and 3H11-12) have been obtained from MCLAB (South San Francisco, Calif.). Both antibodies are tested to determine which one produces the most effective CAR in assays described below. As shown below, second or third (FIG. 23) generation CAR vectors are constructed consisting of the following tandem genes: a kozak consensus sequence; the CD8 signal peptide; the anti-HLA-G heavy chain variable region; a (Glycine4Serine)3 flexible polypeptide linker; the respective anti-HLA-G light chain variable region; CD8 hinge and transmembrane domains; and the CD28, 4-1BB, and CD3 intracellular co-stimulatory signaling domains. Hinge, transmembrane, and signaling domain DNA sequences are ascertained from a patent by Carl June (see US 20130287748 A1). Anti-HLA-G CAR genes are synthesized by Genewiz, Inc. (South Plainfield, N.J.) within a pUC57 vector backbone containing the bla gene, which confers ampicillin resistance to the vector host.

Subcloning of CAR Genes into Lentiviral Plasmids

NovaBlue Singles™ chemically-competent E. coli cells are transformed with anti-HLA-G plasmid cDNA. Following growth of the transformed E. coli cells, the CAR plasmids are purified and digested with the appropriate restriction enzymes to be inserted into an HIV-1-based lentiviral vector containing HIV-1 long terminal repeats (LTRs), packaging signal (Ψ), EF1α promoter, internal ribosome entry site (IRES), and woodchuck hepatitis virus post-transcriptional regulatory element (WPRE) via overnight T₄ DNA ligase reaction (New England Biosciences; Ipswich, Mass.). NovaBlue Singles™ chemically-competent E. coli cells will then be transformed with the resulting anti-HLA-G containing lentiviral plasmid.

### Production of Lentiviral Particles

Prior to transfection, HEK293T cells are seeded at 4.0×10⁶ cells/100 mm tissue-culture-treated plate in 10 mL complete-Tet-DMEM and incubated overnight at 37° C. in a humidified 5% CO₂ incubator. Once 80-90% confluent, HEK293T cells are co-transfected with CAR-gene lentiviral plasmids and lentiviral packaging plasmids containing genes necessary to form lentiviral envelope & capsid components, in addition to a proprietary reaction buffer and polymer to facilitate the formation of plasmid-containing nanoparticles that bind HEK293T cells. After incubating transfected-HEK293T cell cultures for 4 hours at 37° C., the transfection medium is replaced with 10 mL fresh complete Tet DMEM. HEK293T cells will then be incubated for an additional 48 hours, after which cell supernatants are harvested and tested for lentiviral particles via sandwich ELISA against p24, the main lentiviral capsid protein. Lentivirus-containing supernatants are aliquoted and stored at −80° C. until use for transduction of target CD4⁺ and CD8⁺ T cells.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. MACS CD4⁺ and CD8⁺ MicroBeads (Miltenyi Biotec; San Diego, Calif.) kits are used to isolate these human T-cell subsets using magnetically activated LS columns to positive select for CD4⁺ and CD8⁺ T-cells. Magnetically-bound T-cells are then removed from the magnetic MACS separator, flushed from the LS column, and washed in fresh complete medium. The purity of CD4⁺ and CD8⁺ T-cell populations are assessed by flow cytometry using Life Technologies Acoustic Attune® Cytometer, and are enriched by Fluorescence-Activated Cell Sorting performed at USC's flow cytometry core facilities if needed. CD4⁺ and CD8⁺ T-cells are maintained at a density of 1.0×10⁶ cells/mL in complete medium supplemented with 100 IU/mL IL-2 in a suitable cell culture vessel, to which α-CD³/α-CD28 Human T-cell Dynabeads (Life Technologies; Carslbad, Calif.) are added to activate cultured T cells. T-cells are incubated at 37° C. in a 5% CO₂ incubator for 2 days prior to transduction with CAR-lentiviral particles.

Lentiviral Transduction of CD4⁺ CD8⁺ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL complete medium. To various wells, HLA-G CAR-containing lentiviral particles are added to cell suspensions at varying multiplicity of infections (MOIs), such as 1, 5, 10, and 50. Polybrene, a cationic polymer that aids transduction by facilitating interaction between lentiviral particles and the target cell surface, are added at a final concentration of 4 μg/mL. Plates are centrifuged at 800×g for 1 hr at 32° C. Following centrifugation, lentivirus-containing medium are aspirated and cell pellets are resuspended in fresh complete medium with 100 IU/mL IL-2. Cells are placed in a 5% CO₂ humidified incubator at 37° C. overnight. Three days post-transduction, cells are pelleted and resuspended in fresh complete medium with IL-2 and 400 μg/mL Geneticin (G418 sulfate) (Life Technologies; Carlsbad, Calif.). HLA-G CAR modified T-cells are assessed by flow cytometry and southern blot analysis to demonstrate successful transduction procedures. Prior to in vitro and in vivo assays, HLA-G CAR T-cells are enriched by FACS and mixed 1:1 for the in vivo studies.

### In Vitro Assessment of CAR Efficacy by Calcein-Release Cytotoxicity Assays

HLA-G antigen positive and negative human cell lines are collected, washed, and resuspended in complete medium at a concentration of 1.0×10⁶ cells/mL. Calcein-acetoxymethyl (AM) are added to target cell samples at 15 μM, which will then be incubated at 37° C. in a 5% CO₂ humidified incubator for 30 minutes. Dyed positive and negative target cells are washed twice and resuspended in complete medium by centrifugation and added to a 96-well plate at 1.0×10⁴ cells/well. HLA-G CAR T-cells are added to the plate in complete medium at effector-to-target cell ratios of 50:1, 5:1, and 1:1. Dyed-target cells suspended in complete medium and complete medium with 2% triton X-100 will serve as spontaneous and maximal release controls, respectively. The plates are centrifuged at 365×g and 20° C. for 2 minutes before being placed back in the incubator 3 hours. The plates are then centrifuged 10 minutes and cell supernatants are aliquoted to respective wells on a black polystyrene 96-well plate and assessed for fluorescence on a Bio-Tek® Synergy™ HT microplate reader at excitation and emissions of 485/20 nm and 528/20 nm, respectively.

### Quantification of Human Cytokines by Luminex Bioassay.

Supernatants of HLA-G CAR modified T-cells and HLA-G positive and negative tumor cell lines are measured for cytokine secretion as a measure of CAR T-cell activation using standard procedures performed routinely in the laboratory. Data are compared to medium alone and to cultures using non-activated human T-cells to identify background activity. The concentration of IL-2, IFN-g, IL-12, and other pertinent cytokines are measured over time during the incubation process.

In Vivo Assessment of CAR T-Cell Efficacy in Two Xenograft HLA-G Positive Cancer Models

HLA-G CAR T-cells are further evaluated in vivo using two different human tumor cell line xenograft tumor models. For both, solid tumors are established subcutaneously in 6-8 week old female nude mice by injection of 5×10⁶ HLA-G positive or HLA-G negative solid tumor cell lines. When the tumors reach 0.5 cm in diameter, groups of mice (n=5) are treated intravenously with 1 or 3×10⁷ human T-cells as negative controls or HLA-G CAR T-cells constructed from the most active HLA-G antibodies based upon the in vitro study results. Tumor volumes will then be measured by caliper 3×/week and volume growth curves are generated to demonstrate the effectiveness of experimental treatments over controls.

HLA-G is found to be an outstanding target for CAR T-cell development to treat human solid tumors that lose their expression of HLA-A,B,C to avoid immune recognition. It has minimal expression in normal tissues with the exception of the placenta in pregnancy and, therefore, should have very limited off-target positivity and toxicity in patients.

### Example 9

### Anti-HLA-G CAR T-Cells

### Construction of the CAR Lentiviral Constructs

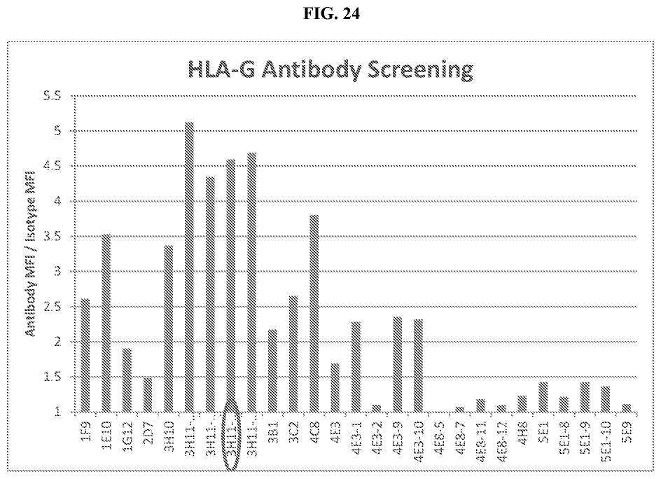

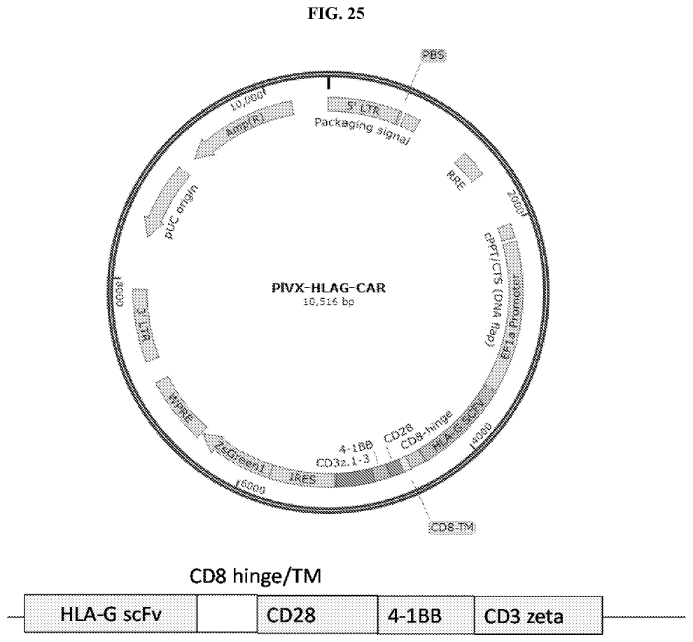

The CAR consists of an extracellular antigen binding moiety or scFV which binds specifically to HLA-G. The scFV is connected via a CD8 hinge region to the cytoplasmic signaling domain, comprised of the CD8 transmembrane region, and the signaling domains from CD28, 4-1BB and CD3z (FIG. 25). The scFV sequence including the signaling domains, were synthetically synthesized by Genewiz Gene Synthesis services (Piscataway, N.J.). The plasmids are purified and digested with the appropriate restriction enzymes to be inserted into an HIV-1-based bicistronic lentiviral vector (pLVX-IRES-ZsGreen, Clontech, Signal Hill, Calif.) containing HIV-1 5′ and 3′ long terminal repeats (LTRs), packaging signal (Ψ), EF1α promoter, internal ribosome entry site (IRES), woodchuck hepatitis virus post-transcriptional regulatory element (WPRE) and simian virus 40 origin (SV40) via overnight T₄ DNA ligase reaction (New England Biosciences; Ipswich, Mass.). NovaBlue Singles™ chemically-competent E. coli cells are then transformed with the resulting CAR-containing lentiviral plasmid.

### Production of Lentiviral Particles

Prior to transfection, HEK 293T cells are seeded at 4.0×106 cells in a 150 cm2 tissue-culture-treated flask in 20 mL DMEM supplemented with 10% dialysed FCS and incubated overnight at 37° C. in a humidified 5% CO2 incubator. Once 80-90% confluent, HEK 293T cells are incubated in 20 ml DMEM supplemented with 1-% dialyzed FCS without penicillin/streptamycin for two hours in at 37° C. in a humidified 5% CO2 incubator. HEK293T cells are co-transfected with the pLVX-B7-H4-CAR plasmid and lentiviral packaging plasmids containing genes necessary to form the lentiviral envelope & capsid components. A proprietary reaction buffer and polymer to facilitate the formation of plasmid-containing nanoparticles that bind HEK 293T cells are also added. After incubating the transfected-HEK 293T cell cultures for 24 hours at 37° C., the transfection medium is replaced with 20 mL fresh complete DMEM. Lentivirus supernatants are collected every 24 hours for three days and the supernatants are centrifuged at 1,250 rpm for 5 mins at 4° C., followed by filter sterilization and centrifugation in an ultracentrifuge at 20,000 g for 2 hrs at 4° C. The concentrated lentivirus is re-suspended in PBS supplemented with 7% trehalose and 1% BSA. The lentivirus is then stored in aliquots at −80° C. until used for transduction of target CD4+ and CD8+ T cells. The cell supernatants harvested after 24 hours are tested for lentiviral particles via sandwich ELISA against p24, the main lentiviral cased protein. Transfection efficiency was estimated between 30%-60% as determined by the visualization of the fluorescent protein marker ZsGreen, under a fluorescent microscope.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. T-cell enrichment kits (Stem Cell Technologies) are used to isolate these human T-cell subsets magnetically using negative selection for CD4+ and CD8+ T-cells. The purity of CD4+ and CD8+ T-cell populations are assessed by flow cytometry using Life Technologies Acoustic Attune® Cytometer, and are enriched by Fluorescence-Activated Cell Sorting. CD4+ and CD8+ T-cells mixed 1:1 are maintained at a density of 1.0×106 cells/mL in complete 50% Click's medium/50 RPMI-1640 medium supplemented with 100 IU/mL IL-2 in a suitable cell culture vessel, to which α-CD³/α-CD28 Human T-cell activator beads (Stem Cell Technologies) are added to activate cultured T cells. T-cells are then incubated at 37° C. in a 5% CO2 incubator for 2 days prior to transduction with CAR lentiviral particles.

Lentiviral Transduction of CD4⁺ CD8⁺ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL in complete medium. Cells are transduced with the lentiviral particles supplemented with Lentiblast, a transfection aid (Oz Biosciences, San Diego, Calif.) to the cells. Transduced cells are then incubated for 24 hours at 37° C. in a humidified 5% CO₂ incubator. The cells are spun down and the media changed, followed by addition of the T-cell activator beads (Stem Cell Technologies, San Diego, Calif.).

### Cell Cytotoxicity Assays

Cytotoxicity of the CAR T-cells is determined using the lactate dehydrogenase (LDH) cytotoxicity kit (Thermo Scientific, Carlsbad, Calif.). Activated T-cells are collected and 1×106 cells are transduced with the HLA-G CAR lentiviral construct as described above. Cells are activated used the T-cell activator beads (Stem Cell Technologies, San Diego, Calif.) for two days prior to cytotoxicity assays. The optimal number of target cells is determined as per the manufacturer's protocol. For the assays, the appropriate target cells are plated in triplicate in a 96 well plate for 24 hours at 37° C. in a 5% CO2 incubator, followed by addition of activated CAR T-cells in ratios of 20:1, 10:1, 5:1 and 1:1, and incubated for 24 hours at 37° C. in a 5% CO2 incubator. Cells are lysed at 37° C. for 45 mins and centrifuged at 1,250 rpm for 5 mins. The supernatants are then transferred to a fresh 96 well plate, followed by the addition of the reaction mixture for 30 mins. The reaction is stopped using the stop solution and the plate read at 450nm with an absorbance correction at 650 nm.

### Western Blotting

T-cells expressing the HLA-CAR are lysed using RIPA buffer. Protein concentrations are estimated by the Bradford Method. Fifty microgram of the protein lysate are run on a 12% reducing poly-acrylamide gel, followed by transfer to a nitrocellulose membrane. The membranes are blocked for an hour in 5% non-fat milk in TBS supplemented with 0.05% Tween. The membranes are then incubated overnight using an antibody specific for CD3ζ (1:250) at 4° C. After three washes, the membranes are incubated in secondary antibody and the bands detected using chemiluminescence. The membranes are stripped and re-probed for β-actin.

### In Vivo Tumor Regression Assay

Foxn1 null mice will be injected with the malignant ovarian cancer cell line, SKOV3, which expresses HLA-G. Two ×106 SKOV3 cells in 200 ul of phosphate buffered saline (PBS) are injected into the left flank of the mice using a 0.2 mL inoculum. T-cells are activated for 2 days with the αCD3/CD28 activator complex (Stem Cell Technologies, San Diego, Calif.). The activated T-cells are then transduced with HLA-G CAR lentiviral particles, followed by activation with the αCD3/CD28 activator complex for an additional 2 days. The activated T-cells expressing the HLA-G CAR (2.5×106) are injected into the mice on day 7 after tumor inoculation. Tumor sizes are assessed twice a week using Vernier calipers and the volume calculated.

### Cytotoxicity for HLA-GCAR T-Cells

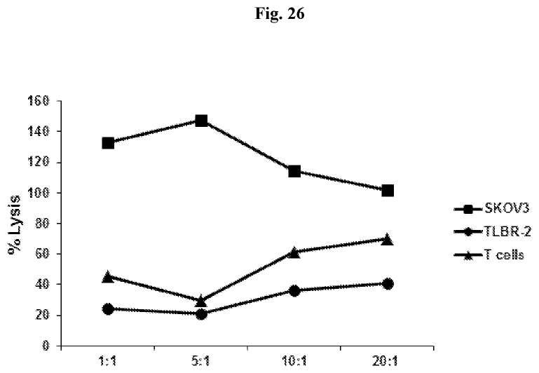

The cytolytic activity of the HLA-G CAR T-cells was examined using SKOV3, an ovarian cell line (FIG. 26). SKOV3 expresses HLA-G, as determined by FACS analysis. HLA-G CAR T-cells were added to the SKOV3 in ratios of 20:1, 10:1, 5:1 and 1:1 of effector to target cells. At a ratio of 10:1, HLA-G CAR T-cells show increased lysis of the target SKOV3 cells with a lysis rate of 42%. In comparison, untransduced T-cells did not lyse SKOV3 cells at any of the ratios tested.

### Protein Expression for HLA-G CAR

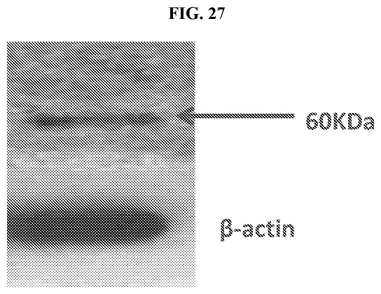

T-cells transduced with the HLA-G CAR express the protein for the CAR as shown by western blotting (FIG. 27). The estimated size of the CAR is around 60 kDA. β-actin was used as a loading control. A CD3ζ antibody which targets the signaling domain used for the CAR was used to detect the CAR protein.

### Example 10

### Generation of Mouse Anti-Human HLA-DR Monoclonal Antibodies

### Antigen

Raji African Burkitt's lymphoma cell nuclei were used as the antigen for producing the Lym-1 antibody. CLL biopsy cell nuclei were used as the antigen for producing the Lym-2 antibody.

### Immunization Procedures

Four week old female BALB/c mice purchased from Harlan Laboratories were immunized every two weeks ×4 with 10⁷ nuclei emulsified with Complete Freund's Adjuvant (first and second immunization) or incomplete Freund's Adjuvant (third and fourth immunization). Mice were injected intradermally with a total of 10⁷ nuclei/adjuvant divided into three separate spots on the back of the mice per immunization. Ten days after the last immunization, blood samples were obtained and tittered by ELISA procedures on antigen coated plates. Mice showing the highest titers then received a fifth immunization boost intravenously without adjuvant in which 10⁶ nuclei were injected via the lateral tail vein in a 100 μl solution of sterile Phosphate Buffered Saline.

### Generation of Hybridomas

Four days later, these mice were sacrificed and the spleens removed for the hybridoma procedure. After dispersing the splenocytes in a solution of RPMI-1640 medium containing Pen/Strep antibiotics, the splenocytes were fused with murine NSO cells using PEG (Hybri MAX, mol wt 1450, Cat. No: p′7181, Sigma). HAT selection was then used to enable only fused cells to grow. Supernatant from wells with growing hybridoma cells were then screened initially by ELISA against antigen coated plates and secondarily by flow cytometry on HLA-DR positive (Raji) and negative human tumor cell lines (CEM T-cell leukemia). Hybridomas showing a positive and high mean fluorescent index (MFI) were selected for subcloning by limiting dilution methods. Subclones were then retested by flow cytometry, frozen in liquid nitrogen, and expanded in 2 L vessels to before antibody was purified by tandon Protein A or G and ion exchange chromatography methods. Purified antibodies were then vialed and stored at −20° C. until used.

### Flow Cytometry Procedures and Data

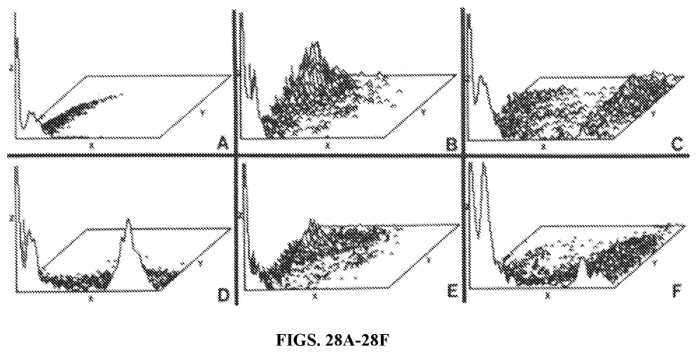

Screening methods using flow cytometry were performed on HLA-DR positive (Raji) and negative (CEM) cell lines using supernatant from hybridomas found positive by ELISA to antigen coated plates. Those hybridomas producing high mean fluorescent indexes (MFI) were then subcloned and rescreened for selective positivity to HLA-DR. As shown below in FIGS. 28A-28F, Lym-1 and Lym-2 produced high MFI to the HLA-DR expressing Raji cell line with a different profile than B1 antibody. From these data, Lym-1 and Lym-2 were selected to generate CAR-T cells as described below.

Immunohistochemistry with Selected Antibodies

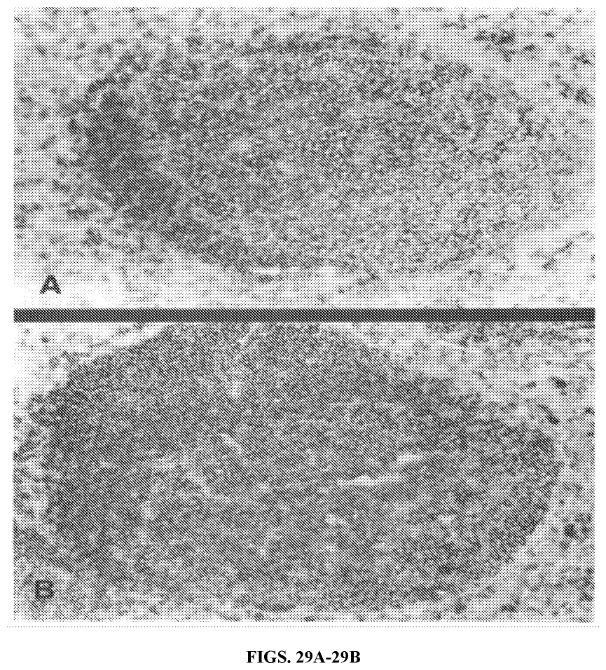

Antibodies Lym-1 and Lym-2 were found to stain HLA-DR positive cells in the germinal centers of human tonsil tissue using standard immunohistochemical procedures and antigen retrieval methods as shown in FIGS. 29A-29B. Staining in thymus, spleen and bone marrow was restricted to B-cell or dendritic cells expressing the HLA-DR antigen (Table 6).

TABLE 6
Reactivity of Lym-1 and Lym-2 with human normal lymphoid
and hematopoietic tissues in frozen sections or cytospins

| | | |
|---|---|---|
| Organ | Lym-1 | Lym-2 |

Lymph node

| | | |
|---|---|---|
| Germinal center | +++^{a} | ++ |
| Mantle zone | + | +++ |
| T-cell zones | − | − |
| Interdigitating histiocytes | ++ | ++ |
| Sinus histiocytes | − | − |
| Endothelium | − | − |

Thymus

| | | |
|---|---|---|
| Cortex | − | − |
| Medulla | ++ Dendritic cells | − |

Spleen

| | | |
|---|---|---|
| White pulp | ++ B-cell zones | ++ B-cell zones |
| Red pulp | − | − |

Bone marrow

| | | |
|---|---|---|
| Myeloid | − | − |
| Erythroid | − | − |
| Megakaryocytes | − | − |

^{a}Intensity of immunoperoxidase staining from − to +++.

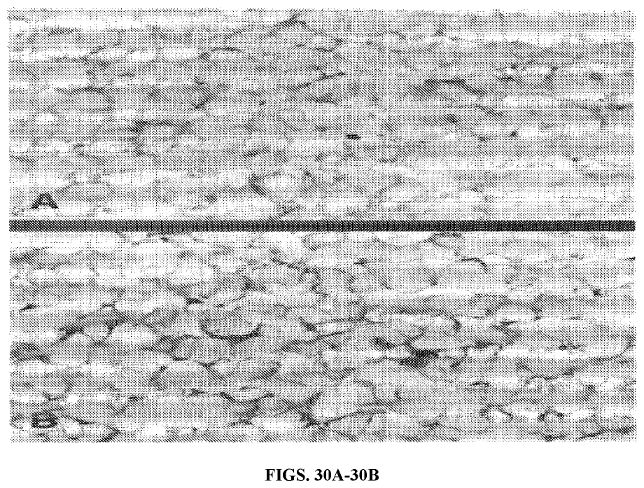

As shown in FIGS. 30A-30B, HLA-DR positivity was seen on the cell membrane of antigen positive tumors such as intermediate grade B-cell lymphomas. Finally, tissue sections from normal tissues and organs showed restricted reactivity to lymphoid B-cells and macrophages of the skin (Table 7). The availability of a companion diagnostic antibody for HLA-DR using immunohistochemistry enables the identification of patients likely to benefit from HLA-DR CAR T-cell therapy in upcoming clinical trials.

TABLE 7
Reactivity of Lym-1 and Lym-2 with normal
non-lymphoid tissues in frozen sections

Reactivity

| Tissue | Lym-1 | Lym-2 |
|---|---|---|
| Adrenal | −^{a} | − |
| Brain | − | − |
| Breast | − | − |
| Cervix | − | − |
| Colon | + surface epithelium | − |
| Duodenum | − | − |
| Heart | − | − |
| Kidney | − | − |
| Liver | − | − |
| Lung | − | − |
| Ovary | − | − |
| Pancreas | − | − |
| Salivary glands | − | − |
| Skin | + macrophages only | − |
| Skeletal muscle | − | − |
| Smooth muscle | − | − |
| Stomach | − | − |
| Testis | − | − |
| Thyroid | − | − |

^{a}Intensity of immunoperoxidase staining from − to +++.

### Live Cell Radioimmunoassay

Using Lym-1 or Lym-2, a panel of human lymphoma and solid tumor cell lines were screened for binding using a live cell radioimmunoassay procedure. For this assay, suspension cultures and solid tumor cell lines which were dislodged from their flasks with EDTA-trypsin were washed twice in cold buffer consisting of PBS, bovine serum albumin (1 mg/ml), and 0.02% sodium azide. Cells (5×10⁵) resuspended in 100 μl of wash buffer were pipetted into microwells pretreated overnight with BSA (10 mg/ml) in PBS to prevent antibody binding to the wells. Lym-1 or Lym-2 supernatant were then added (100 μ/l well) for a 30 minute incubation period with continuous shaking using a microshaker apparatus for 96 well plates at room temperature. After 4 washes, 100,000 cpm of I-125 goat anti-mouse

IgG was then added in 100 μl and incubated with the cells for an additional 30 minute incubation with continuous shaking. After 4 final washes, the wells were counted in a gamma counter to determine antibody binding to each cell preparation. The results of these studies showed that for a large panel of human lymphoma and leukemia biopsies, reactivity of Lym-1 and Lym-2 was restricted to tumors of B-cell but not T-cell origin (Table 8).

TABLE 8
Reactivity of Lym-1 and Lym-2 with human malignant
lymphoma and leukemia biopsy specimens

| | | |
|---|---|---|
| Diagnosis | Lym-1^{a} | Lym-2^{a} |

Lymphomas^{b} (frozen sections of lymph node biopsies^{c})

| | | |
|---|---|---|
| Well-differentiated lymphocytic | 1/3 | 3/3 |
| Poorly differentiated lymphocytic, nodular | 0/2 | 2/2 |
| Poorly differentiated lymphocytic, diffuse | 1/3 | 3/3 |
| Mixed lymphocytic and histiocytic | 8/9 | 7/9 |
| Histiocytic (B-cell) | 12/17 | 12/17 |
| T-cell | 0/2 | 0/2 |

Leukemias (cytospins of peripheral blood^{d})
Chronic lymphocytic

| | | |
|---|---|---|
| B-cell type | 4/10 | 8/10 |
| T-cell type | 0/5 | 0/5 |

^{a}Positive/total.
^{b}Rappaport classification.
^{c}Immunoperoxidase technique.
^{d}Indirect immunofluorescence.

Consistent with these results, Lym-1 and Lym-2 was found to bind to a select number of human lymphoma and leukemia cell lines as shown in Table 9.

TABLE 9
Reactivity of Lym-1 and Lym-2 with human malignant
lymphoma cell lines by live cell radioimmunoassay

| | | |
|---|---|---|
| Cell Line | Lym-1 | Lym-2 |

Burkitt's Lymphoma

| | | |
|---|---|---|
| Raji | ++++^{a} | ++ |
| EB3 | − | − |
| DG-75 | ++++ | ++++ |
| NK-9 | ++ | ++++ |
| AL-1 | − | + |
| Daudi | + | +++ |
| NU-AmB-1 | + | ++ |
| SU-AmB-1 | − | + |
| SU-AmB-2 | − | − |
| RAMOS | − | − |
| Chevallier | ++++ | − |
| B46M | + | + |
| B35M | ++++ | ++++ |
| DND-39 | + | − |
| U-698-M | + | ++ |
| HRIK | − | + |

Large Cell Lymphoma

| | | |
|---|---|---|
| SU-DHL-1 | − | − |
| SU-DHL-2 | − | − |
| SU-DHL-4 | − | ++++ |
| SU-DHL-5 | + | ++ |
| SU-DHL-6 | +++ | +++ |
| SU-DHL-7 | + | − |
| SU-DHL-8 | + | − |
| SU-DHL-9 | + | + |
| SU-DHL-10 | − | ++++ |
| SU-DHL-16 | − | − |
| NU-DHL-1 | ++++ | − |
| U-937 | − | − |

Undifferentiated lymphoma

| | | |
|---|---|---|
| NU-DUL-1 | − | + |

^{a}−, <2,000 cpm; +, 2,000-6,000cpm; ++, 6,000-10,000 cpm; +++, 10,000-15,000 cpm; ++++, >15,000 cpm.

By contrast, Lym-1 and Lym-2 was not found to bind to 35 human solid tumor cell lines using live cell radioimmunoassay procedures described above (Table 10).

TABLE 10
Reactivity of Lym-1 and Lym-2 with 35 human solid
tumor cell lines by live cell radioimmunoassay

| Cell line | Derivation | Lym-1 | Lym-2 |
|---|---|---|---|
| 734B | Breast carcinoma | −^{a} | − |
| 578T | Breast carcinoma | − | − |
| C-399 | Colon carcinoma | − | − |
| Hutu-80 | Colon carcinoma | − | − |
| HT-29 | Colon carcinoma | − | − |
| HeLa | Cervical carcinoma | − | − |
| SW 733 | Papillary carcinoma of bladder | − | − |
| SW 780 | Transitional cell carcinoma of bladder | − | − |
| SW 451 | Squamous cell carcinoma of esophagus | − | − |
| SW 579 | Squamous cell carcinoma of thyroid | − | − |
| SW 156 | Hypernephroma | − | − |
| 60 | Small cell carcinoma of lung | − | − |
| 464 | Small cell carcinoma of lung | − | − |
| NCI-H69 | Small cell carcinoma of lung | − | − |
| 125 | Adenocarcinoma of lung | − | − |
| A427 | Adenocarcinoma of lung | − | − |
| A549 | Adenocarcinoma of lung | − | − |
| SW 1503 | Mesothelioma | − | − |
| BM 166 | Neuroblastoma | − | − |
| IMR-5 | Neuroblastoma | − | − |
| Y79 | Retinoblastoma | − | − |
| A172 | Astrocytoma | − | − |
| SW 608 | Astrocytoma | − | − |
| U118 MG | Glioblastoma | − | − |
| NU-04 | Glioblastoma | − | − |
| CaCl 74-36 | Melanoma | − | − |
| Colo 38 | Melanoma | − | − |
| SW 872 | Liposarcoma | − | − |
| HS 919 | Liposarcoma | − | − |
| SW 1045 | Synovial sarcoma | − | − |
| SW 80 | Rhabdomyoscarcoma | − | − |
| SW 1353 | Chondrosarcoma | − | − |
| 4-998 | Osteogenic sarcoma | − | − |
| 4-906 | Osteogenic sarcoma | − | − |
| SU-CCS-1 | Clear cell sarcoma | − | − |

^{a}−, <2,000 cpm; +, 2,000-6,000cpm; ++, 6,000-10,000 cpm; +++, 10,000-15,000 cpm; ++++, >15,000 cpm.

### Binding Profiles of Lym-1 and Lym-2 Antibodies and Identification of Lym-1 Antigen

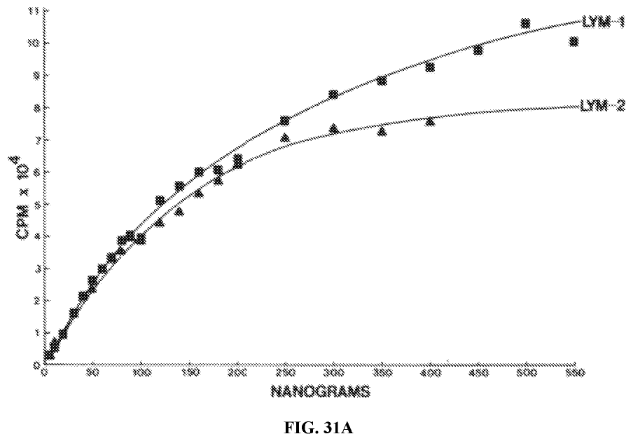

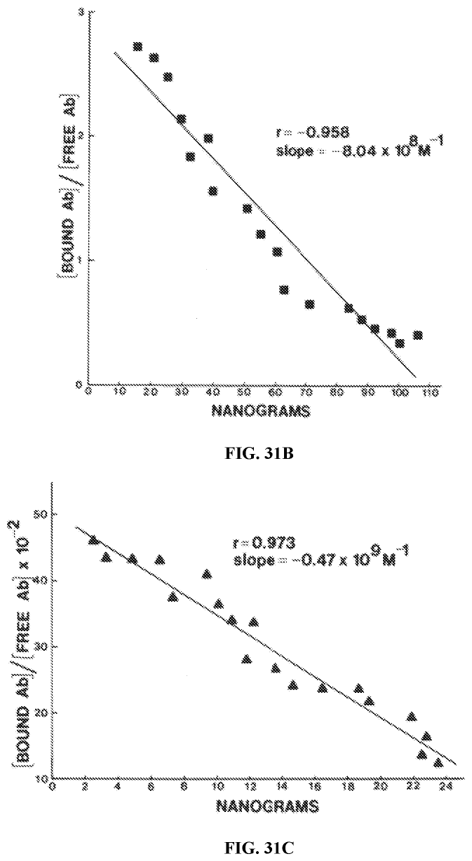

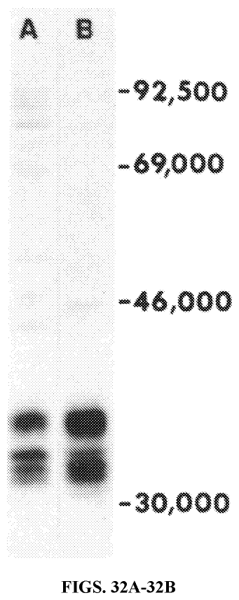

Binding profiles and Scatchard plot analyses of Lym-1 binding with Raji cells is shown in FIG. 31A. Likewise, Scatchard plot analyses of Lym-2 binding with the ARH-77 myeloma cell line are shown in FIG. 31B. These data demonstrated that both antibodies have 10⁸ M⁻¹binding affinities to antigen positive tumor cell lines. As shown in Table 11, when compared to normal peripheral blood B cells, there was a two to four-fold decrease in binding affinities compared to that seen with tumor cells. In addition, metabolic labeling of Raji cells with ³⁵S-methionine and ¹⁴C-leucine showed the characteristic banding pattern seen for HLA-DR (FIGS. 32A-32B). As a control, the SC-1 anti-HLA-DR antibody was used in parallel and gave the same banding pattern with identical protein molecular weights by SDS-gel electrophoresis.

TABLE 11
Avidity constants of Lym-1 and Lym-2 using target tumor
cell lines (Raji, ARH-77) and tonsil lymphocytes

| Monoclonal antibody | Tumor cell line | Tonsil |
|---|---|---|
| Lym-1 | 4.02 × 10⁸ M⁻¹ | 0.88 × 10⁸ M⁻¹ |
| Lym-2 | 2.33 × 10⁸ M⁻¹ | 1.23 × 10⁸ M⁻¹ |

### Example 11

### Generation of HLA-DR CAR T-Cells

### Construction and Synthesis Single Chain HLA-DR Antibody Genes

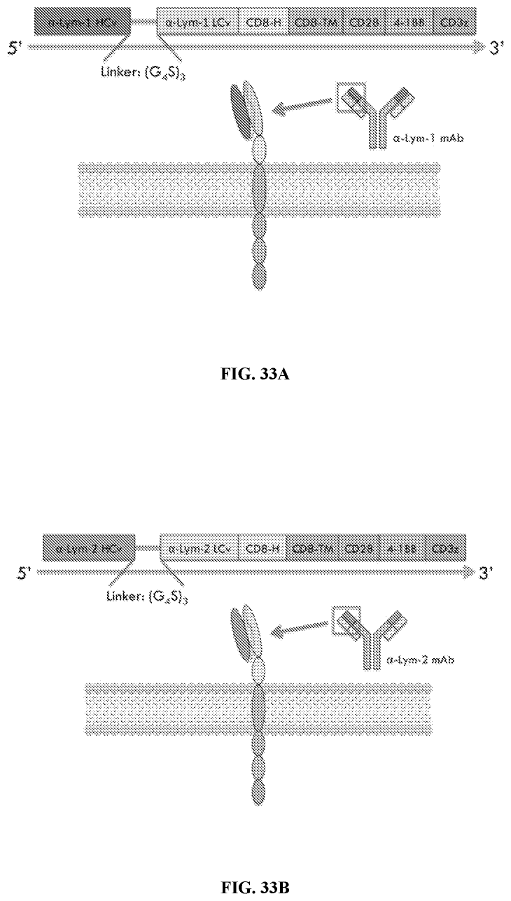

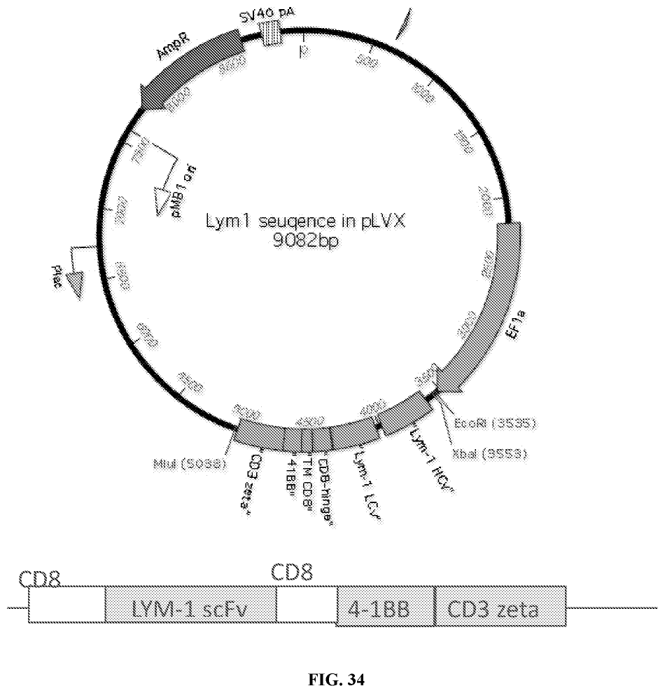

The DNA sequences for 2 high binding anti-HLA-DR antibodies generated in the laboratory (Lym-1 and Lym-2) are obtained from MCLAB (South San Francisco, Calif.). Both antibodies are tested to determine which one produces the most effective CAR in assays described below. As shown below, second or third (FIG. 33) generation CAR vectors are constructed consisting of the following tandem genes: a kozak consensus sequence; the CD8 signal peptide; the anti-HLA-DR heavy chain variable region; a (Glycine4Serine)3 flexible polypeptide linker; the respective anti-HLA-DR light chain variable region; CD8 hinge and transmembrane domains; and the CD28, 4-1BB, and CD3 intracellular co-stimulatory signaling domains. Hinge, transmembrane, and signaling domain DNA sequences are ascertained from a patent by Carl June (see U.S. Patent Application Publication No. 2013/0287748 A1). Anti-HLA-DR CAR genes are synthesized by Genewiz, Inc. (South Plainfield, N.J.) within a pUC57 vector backbone containing the bla gene, which confers ampicillin resistance to the vector host.

Subcloning of CAR Genes into Lentiviral Plasmids

NovaBlue Singles™ chemically-competent E. coli cells are transformed with anti-HLA-DR plasmid cDNA. Following growth of the transformed E. coli cells, the CAR plasmids are purified and digested with the appropriate restriction enzymes to be inserted into an HIV-1-based lentiviral vector containing HIV-1 long terminal repeats (LTRs), packaging signal (Ψ), EF1α promoter, internal ribosome entry site (IRES), and woodchuck hepatitis virus post-transcriptional regulatory element (WPRE) via overnight T₄ DNA ligase reaction (New England Biosciences; Ipswich, Mass.). NovaBlue Singles™ chemically-competent E. coli cells are then transformed with the resulting anti-HLA-DR containing lentiviral plasmid.

### Production of Lentiviral Particles

Prior to transfection, HEK293T cells are seeded at 4.0×10⁶ cells/100 mm tissue-culture-treated plate in 10 mL complete-Tet-DMEM and incubated overnight at 37° C. in a humidified 5% CO₂ incubator. Once 80-90% confluent, HEK293T cells are co-transfected with CAR-gene lentiviral plasmids and lentiviral packaging plasmids containing genes necessary to form lentiviral envelope & capsid components, in addition to a proprietary reaction buffer and polymer to facilitate the formation of plasmid-containing nanoparticles that bind HEK293T cells. After incubating transfected-HEK293T cell cultures for 4 hours at 37° C., the transfection medium is replaced with 10 mL fresh complete Tet DMEM. HEK293T cells are then incubated for an additional 48 hours, after which cell supernatants are harvested and tested for lentiviral particles via sandwich ELISA against p24, the main lentiviral capsid protein. Lentivirus-containing supernatants are aliquoted and stored at −80° C. until use for transduction of target CD4⁺ and CD8⁺ T cells.

Purification, Activation, and Enrichment of Human CD4⁺ and CD8⁺ Peripheral Blood T-Cells

Peripheral blood mononuclear cells (PBMCs) are enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. MACS CD4⁺ and CD8⁺ MicroBeads (Miltenyi Biotec; San Diego, Calif.) kits are used to isolate these human T-cell subsets using magnetically activated LS columns to positive select for CD4⁺ and CD8⁺ T-cells. Magnetically-bound T-cells are then removed from the magnetic MACS separator, flushed from the LS column, and washed in fresh complete medium. The purity of CD4⁺ and CD8⁺ T-cell populations are assessed by flow cytometry using Life Technologies Acoustic Attune® Cytometer, and are enriched by Fluorescence-Activated Cell Sorting performed at USC's flow cytometry core facilities if needed. CD4⁺ and CD8⁺ T-cells are maintained at a density of 1.0×10⁶ cells/mL in complete medium supplemented with 100 IU/mL IL-2 in a suitable cell culture vessel, to which α-CD³/α-CD28 Human T-cell Dynabeads (Life Technologies; Carslbad, Calif.) are added to activate cultured T cells. T-cells are incubated at 37° C. in a 5% CO₂ incubator for 2 days prior to transduction with CAR-lentiviral particles.

Lentiviral Transduction of CD4⁺ CD8⁺ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL complete medium. To various wells, HLA-DR CAR-containing lentiviral particles are added to cell suspensions at varying multiplicity of infections (MOIs), such as 1, 5, 10, and 50. Polybrene, a cationic polymer that aids transduction by facilitating interaction between lentiviral particles and the target cell surface, are added at a final concentration of 4 μg/mL. Plates are centrifuged at 800×g for 1 hr at 32° C. Following centrifugation, lentivirus-containing medium are aspirated and cell pellets are resuspended in fresh complete medium with 100 IU/mL IL-2. Cells are placed in a 5% CO₂ humidified incubator at 37° C. overnight. Three days post-transduction, cells are pelleted and resuspended in fresh complete medium with IL-2 and 400 μg/mL Geneticin (G418 sulfate) (Life Technologies; Carlsbad, Calif.). HLA-DR CAR modified T-cells are assessed by flow cytometry and southern blot analysis to demonstrate successful transduction procedures. Prior to in vitro and in vivo assays, HLA-DR CAR T-cells are enriched by FACS and mixed 1:1 for the in vivo studies.

### In Vitro Assessment of Car Efficacy by Calcein-Release Cytotoxicity Assays

HLA-DR antigen positive and negative human cell lines are collected, washed, and resuspended in complete medium at a concentration of 1.0×10⁶ cells/mL. Calcein-acetoxymethyl (AM) are added to target cell samples at 15 which are then incubated at 37° C. in a 5% CO₂ humidified incubator for 30 minutes. Dyed positive and negative target cells are washed twice and resuspended in complete medium by centrifugation and added to a 96-well plate at 1.0×10⁴ cells/well. HLA-DR CAR T-cells are added to the plate in complete medium at effector-to-target cell ratios of 50:1, 5:1, and 1:1. Dyed-target cells suspended in complete medium and complete medium with 2% triton X-100 serve as spontaneous and maximal release controls, respectively. The plates are centrifuged at 365×g and 20° C. for 2 minutes before being placed back in the incubator 3 hours. The plates are then centrifuged 10 minutes and cell supernatants are aliquoted to respective wells on a black polystyrene 96-well plate and assessed for fluorescence on a Bio-Tek® Synergy™ HT microplate reader at excitation and emissions of 485/20 nm and 528/20 nm, respectively.

### Quantification of Human Cytokines by Luminex Bioassay

Supernatants of HLA-DR CAR modified T-cells and HLA-DR positive and negative tumor cell lines are measured for cytokine secretion as a measure of CAR T-cell activation using standard procedures performed routinely in the laboratory. Data are compared to medium alone and to cultures using non-activated human T-cells to identify background activity. The concentration of IL-2, IFN-g, IL-12, and other pertinent cytokines are measured over time during the incubation process.

### In Vivo Assessment of CAR T-Cell Efficacy in Two Xenograft HLA-DR Positive Cancer Models

HLA-DR CAR T-cells are further evaluated in vivo using two different human tumor cell line xenograft tumor models. For both, solid tumors are established subcutaneously in 6-8 week old female nude mice by injection of 5×10⁶ HLA-DR positive or HLA-DR negative solid tumor cell lines. When the tumors reach 0.5 cm in diameter, groups of mice (n=5) are treated intravenously with 1 or 3×10⁷ human T-cells as negative controls or HLA-DR CAR T-cells constructed from the most active HLA-DR antibodies based upon the in vitro study results. Tumor volumes are then measured by caliper 3×/week and volume growth curves are generated to demonstrate the effectiveness of experimental treatments over controls.

HLA-DR is found to be an outstanding target for CAR T-cell development.

### Example 12

### Lym-1 CAR Cells

### Construction of the CAR Lentiviral Constructs

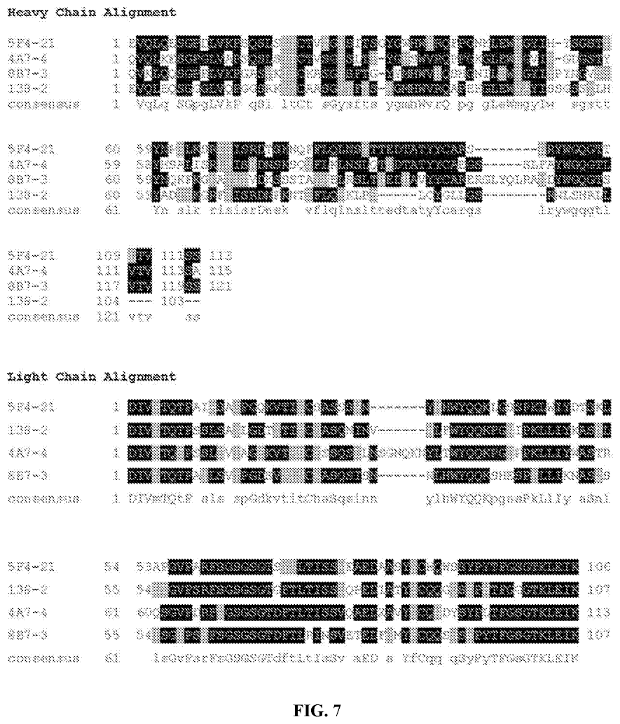

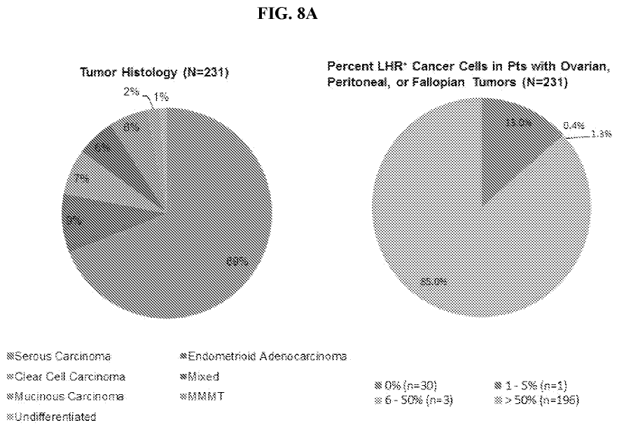

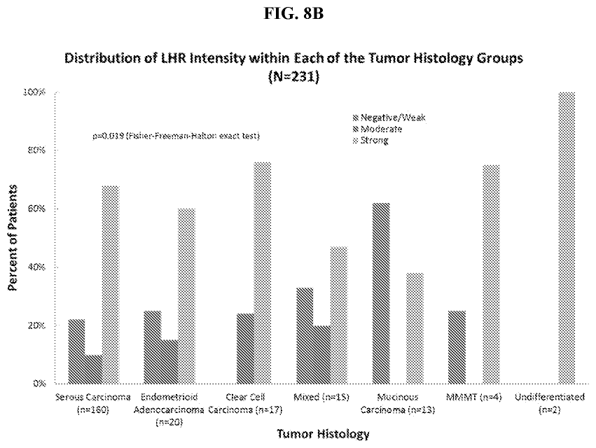

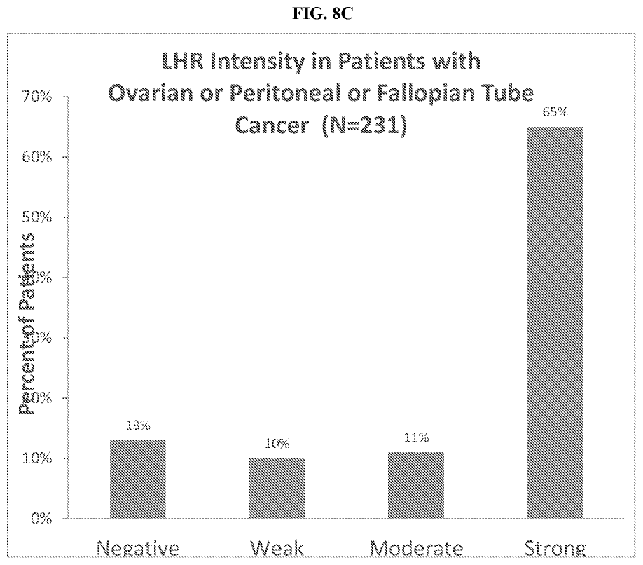

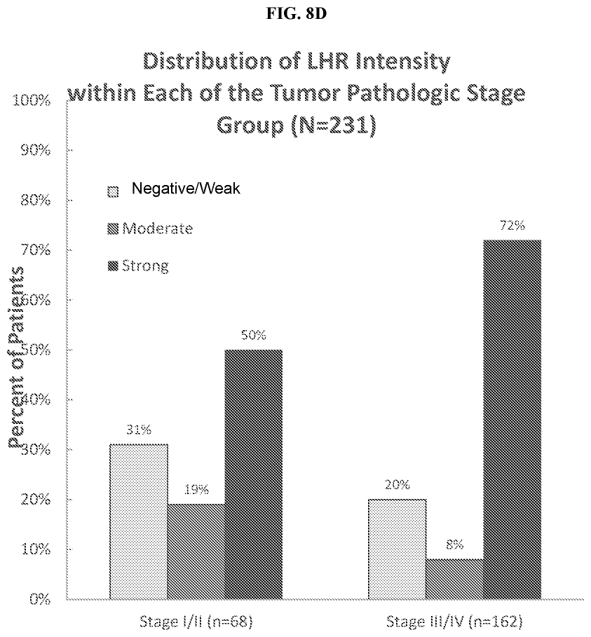

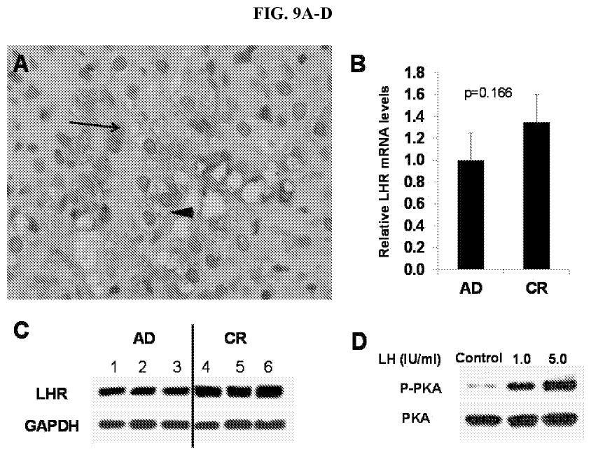

The Lym-1 CAR vector contains a CD8 leader sequence followed by the extracellular antigen binding moiety or scFV, which binds specifically to Lym-1 antigen. The scFV is connected via a CD8 hinge region to the cytoplasmic signaling domain, comprised of the CD8 transmembrane region, and the signaling domains from 4-1BB and CD3 (FIG. 7). The CAR sequence including the signaling domains, were synthetically synthesized by Genewiz Gene Synthesis services (Piscataway, N.J.). The plasmids are purified and digested with the appropriate restriction enzymes to be inserted into an HIV-1-based lentiviral vector (pLVX-IRES-ZsGreen, Clontech, Signal Hill, Calif.) containing HIV-1 5′ and 3′ long terminal repeats (LTRs), packaging signal (Ψ), EF1α promoter, internal ribosome entry site (IRES), woodchuck hepatitis virus post-transcriptional regulatory element (WPRE) and simian virus 40 origin (SV40) via overnight T4 DNA ligase reaction (New England Biosciences; Ipswich, Mass.), followed by deletion of the IRES-ZsGreen using restriction enzyme digestion and ligation with T4 DNA ligase. NovaBlue Singles™ chemically-competent E. coli cells are then transformed with the resulting CAR-containing lentiviral plasmid.

### Production of Lentiviral Particles

Prior to transfection, HEK 293T cells are seeded at 4.0×106 cells in a 150 cm2 tissue-culture-treated flask in 20 mL DMEM supplemented with 10% dialyzed FCS and incubated overnight at 37° C. in a humidified 5% CO2 incubator. Once 80-90% confluent, HEK 293T cells are incubated in 20 ml DMEM supplemented with 1-% dialyzed FCS without penicillin/streptamycin for two hours in at 37° C. in a humidified 5% CO2 incubator. HEK293T cells are co-transfected with the CAR plasmid and lentiviral packaging plasmids containing genes necessary to form the lentiviral envelope & capsid components. A proprietary reaction buffer and polymer to facilitate the formation of plasmid-containing nanoparticles that bind HEK 293T cells are also added. After incubating the transfected-HEK 293T cell cultures for 24 hours at 37° C., the transfection medium is replaced with 20 mL fresh complete DMEM. Lentivirus supernatants are collected every 24 hours for three days and the supernatants are centrifuged at 1,250 rpm for 5 mins at 4° C., followed by filter sterilization and centrifugation in an ultracentrifuge at 20,000 g for 2 hrs at 4° C. The concentrated lentivirus is re-suspended in PBA containing 7% trehalose and 1% BSA. The lentivirus is then aliquoted and stored at −80° C. until use for transduction of target CD4+ and CD8+ T cells. The cell supernatants harvested after 24 hours are tested for lentiviral particles via sandwich ELISA against p24, the main lentiviral capsid protein. Transfection efficiency was estimated between 20%-50%, by staining with a biotin-labeled Protein L antibody (Genscript, Piscataway, N.J.), followed by incubation with a streptavidin conjugated to PE, and detection by FACS analysis.

### Purification, Activation, and Enrichment of Human CD4+ and CD8+ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. T-cell enrichment kits (Stem Cell Technologies) are used to isolate these human T-cell subsets magnetically using negative selection for CD4+ and CD8+ T-cells. The purity of CD4+ and CD8+ T-cell populations is assessed by flow cytometry using Life Technologies Acoustic Attune® Cytometer, and are enriched by Fluorescence-Activated Cell Sorting. CD4+ and CD8+ T-cells mixed 1:1 are maintained at a density of 1.0×106 cells/mL in complete 50% Click's medium/50 RPMI-1640 medium supplemented with 100 IU/mL IL-2 in a suitable cell culture vessel, to which α-CD³/α-CD28 Human T-cell activator beads (Stem Cell Technologies) are added to activate cultured T cells. T-cells are then incubated at 37° C. in a 5% CO2 incubator for 2 days prior to transduction with CAR lentiviral particles.

### Lentiviral Transduction of CD4+ CD8+ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells will be plated at a concentration of 1.0×106 cells/mL in complete medium. Cells will be transduced with the lentiviral particles supplemented with Lentiblast, a transfection aid (Oz Biosciences, San Diego, Calif.) to the cells. Transduced cells were incubated for 24 hours at 37° C. in a humidified 5% CO2 incubator. The cells are spun down and the media changed, followed by addition of the T-cell activator beads (Stem Cell Technologies, San Diego, Calif.).

### Detection of Lym-1 CAR Expression by Flow Cytometry

Seven days after Lentivirus transduction, primary T-cells are washed 3× using wash buffer (4% BSA in PBS). Cells are incubated with Biotein-Protein L (2 ug, Genscript, Piscataway, N.J.) at 4° C. for 45 min. Cells are again washed 3× with wash buffer, followed by incubation with 2 ul of Streptavidin-PE (BD Sciences, La Jolla, Calif.) at 4° C. for 45 min. Cells are washed 3× and analyzed using flow cytometry (Attune Cytometer, Applied Biosciences, Carlsbad, Calif.).

### Cell Cytotoxicity Assays

Cytotoxicity of the Lym-1 CAR T-cells are determined using the lactate dehydrogenase (LDH) cytotoxicity kit (Thermo Scientific, Carlsbad, Calif.). Activated T-cells are collected and 1×106 cells are transduced with the Lym-1 CAR lentiviral construct as described above. Cells are activated used the T-cell activator beads (Stem Cell Technologies, San Diego, Calif.) for two days prior to cytotoxicity assays. The optimal number of target cells is determined as per the manufacturer's protocol. For the assays, the appropriate target cells are plated in triplicate in a 96 well plate for 24 hours at 37° C. in a 5% CO2 incubator, followed by addition of activated CAR T-cells in ratios of 20:1, 10:1, 5:1 and 1:1, and incubated for 24 hours at 37° C. in a 5% CO2 incubator. Cells are lysed at 37° C. for 45 mins and spun down at 1,250 rpm for 5 minutes. The supernatants are transferred to a fresh 96 well plate, followed by the addition of the reaction mixture for 30 minutes. The reaction is stopped using the stop solution and the plate read at 450nm with an absorbance correction at 650 nm.

### In Vivo Tumor Regression Assay

Foxn1 null mice are injected with immortalized B lymphoma cell line, Raji, which expresses the Lym-1 antigen. Two ×106 Raji cells with 1×106 human fibroblasts in 200 ul of phosphate buffered saline (PBS) are injected into the left flank of pre-irradiated mice (400 rads) to reduce the number of circulating NK cells enabling the heterotransplants to implant at a high frequency. T-cells are activated for 2 days with the αCD3/CD28 activator complex (Stem Cell Technologies, San Diego, Calif.). The activated T-cells are then transduced with Lym-1 CAR lentiviral particles, followed by activation with the αCD3/CD28 activator complex for an additional 2 days. The activated T-cells expressing the Lym-1 CAR (2.5×106) are injected intravenously via the lateral tail vein into the mice on day 7 after tumor inoculation. Tumor sizes are assessed 3×/week using Vernier calipers and the tumor volumes calculated.

### Detection of Lym-1 CAR Expression

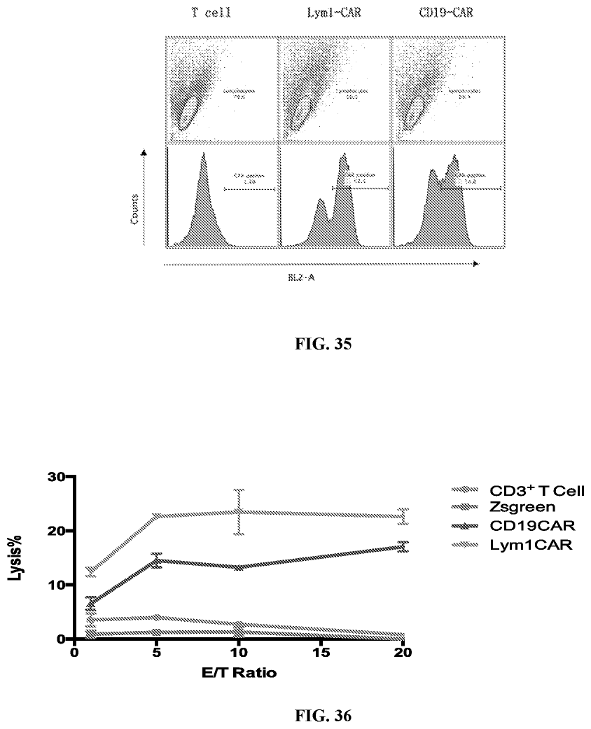

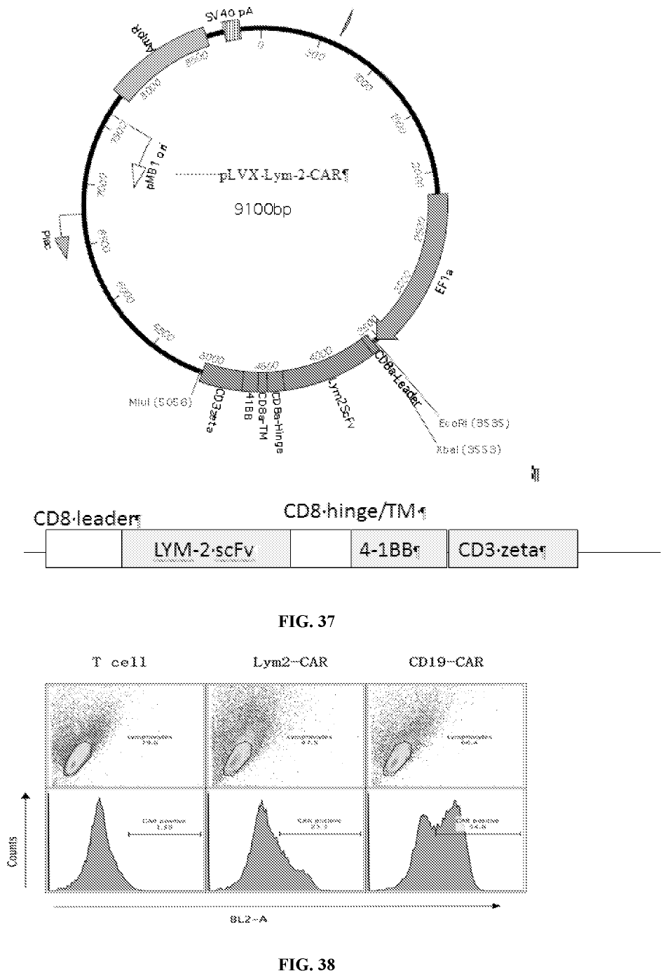

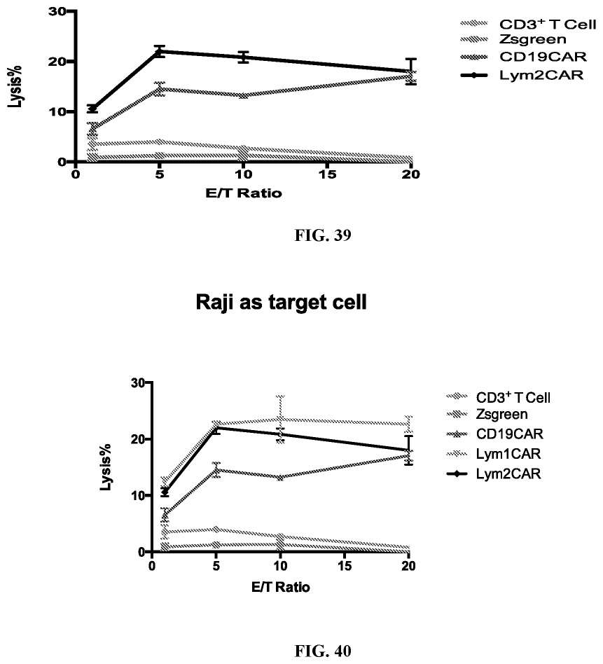

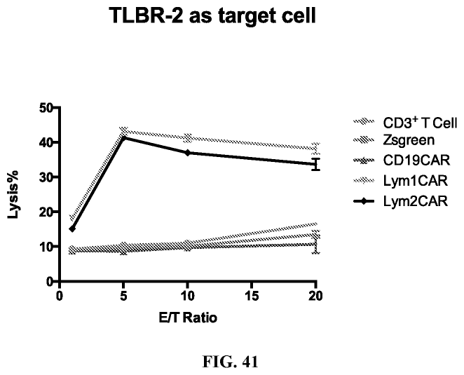

Analysis of the Lym-1 CAR T-cells for expression of the Lym-1 CAR, showed 62.5% of the transduced T-cells positive for Lym-1 (FIG. 35 middle panel). In contrast, only 1% of the un-transduced T-cells used as a control were positive for CAR expression (FIG. 35 left panel). CD19 transduced T-cells were used as a positive control and showed 52% expression of the CD19 CAR (FIG. 35 right panel).

### Cytotoxicity for Lym-1 CAR T-Cells

The cytolytic activity of the Lym-1 CAR T-cells was examined using Raji, a B-cell lymphoma cell line. Raji expresses the Lym-1 antigen (HLA-Dr10), as determined by FACS analysis. Lym-1 CAR T-cells were added to the Raji cells in ratios of 20:1, 10:1, 5:1 and 1:1 of effector to target cells. Lym-1 CAR T-cells showed increased lysis of the target Raji cells at ratios of 5:1, 10:1 and 20:1 with a lysis rate of 22%. In comparison, untransduced T-cells did not lyse Raji cells at any of the ratios tested.

### Example 13

### Lym-2 CAR Cells

### Construction of the CAR Lentiviral Constructs

The Lym-2 CAR vector contains a CD8 leader sequence followed by the extracellular antigen binding moiety or scFV, which binds specifically to the Lym-2 antigen (HLA-Dr). The scFV is connected via a CD8 hinge region to the cytoplasmic signaling domain, comprised of the CD8 transmembrane region, and the signaling domains from 4-1BB and CD3ζ. The CAR sequence including the signaling domains, were synthetically synthesized by Genewiz Gene Synthesis services (Piscataway, N.J.). The plasmids are purified and digested with the appropriate restriction enzymes to be inserted into an HIV-1-based lentiviral vector (pLVX-IRES-ZsGreen, Clontech, Signal Hill, Calif.) containing HIV-1 5′ and 3′ long terminal repeats (LTRs), packaging signal (Ψ), EF1α promoter, internal ribosome entry site (IRES), woodchuck hepatitis virus post-transcriptional regulatory element (WPRE) and simian virus 40 origin (SV40) via overnight T₄ DNA ligase reaction (New England Biosciences; Ipswich, Mass.), followed by deletion of the IRES-ZsGreen using restriction enzyme digestion and ligation with T₄ DNA ligase. NovaBlue Singles™ chemically-competent E. coli cells are then transformed with the resulting CAR-containing lentiviral plasmid.

### Production of Lentiviral Particles

Prior to transfection, HEK 293T cells are seeded at 4.0×10⁶ cells in a 150 cm² tissue-culture-treated flask in 20 mL DMEM supplemented with 10% dialyzed FCS and incubated overnight at 37° C. in a humidified 5% CO₂ incubator. Once 80-90% confluent, HEK 293T cells are incubated in 20 ml DMEM supplemented with 1-% dialyzed FCS without penicillin/streptamycin for two hours in a 37° C. humidified 5% CO₂ incubator. HEK293T cells are co-transfected with the CAR plasmid and lentiviral packaging plasmids containing genes necessary to form the lentiviral envelope & capsid components. A proprietary reaction buffer and polymer to facilitate the formation of plasmid-containing nanoparticles that bind HEK 293T cells are also added. After incubating the transfected-HEK 293T cell cultures for 24 hours at 37° C., the transfection medium is replaced with 20 mL fresh complete DMEM. Lentivirus supernatants are collected every 24 hours for 3 days and the supernatants are centrifuged at 1,250 rpm for 5 mins at 4° C., followed by filter sterilization and centrifugation in an ultracentrifuge at 20,000 g for 2 hrs at 4° C. The concentrated lentivirus is re-suspended in PBA containing 7% trehalose and 1% BSA. The lentivirus is aliquoted and stored at −80° C. until use for transduction of target CD4⁺ and CD8⁺ T cells. The cell supernatants harvested after 24 hours are tested for lentiviral particles via a sandwich ELISA against p24, the main lentiviral capsid protein. Transfection efficiency was estimated between 20%-50%, by staining with a biotin-labeled Protein L antibody (Genscript, Piscataway, N.J.), followed by incubation with a streptavidin conjugated to PE, and detection by FACS analysis.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. T-cell enrichment kits (Stem Cell Technologies) are used to isolate these human T-cell subsets magnetically using negative selection for CD4⁺ and CD8⁺ T-cells. The purity of CD4⁺ and CD8⁺ T-cell populations is assessed by flow cytometry using Life Technologies Acoustic Attune® Cytometer, and will be enriched by Fluorescence-Activated Cell Sorting. CD4⁺ and CD8⁺ T-cells mixed 1:1 are maintained at a density of 1.0×10⁶ cells/mL in complete 50% Click's medium/50 RPMI-1640 medium supplemented with 100 IU/mL IL-2 in a suitable cell culture vessel, to which α-CD³/α-CD28 Human T-cell activator beads (Stem Cell Technologies) are added to activate cultured T cells. T-cells are then incubated at 37° C. in a 5% CO₂ humidified incubator for 2 days prior to transduction with CAR lentiviral particles.

Lentiviral Transduction of CD4⁺ CD8⁺ T-Cells

Activated T-cells are collected and dead cells removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL in complete medium. Cells are transduced with the lentiviral particles supplemented with Lentiblast, a transfection aid (Oz Biosciences, San Diego, Calif.) to the cells. Transduced cells are incubated for 24 hours at 37° C. in a 37° C. humidified 5% CO₂ incubator. The cells are spun down and the media changed, followed by addition of the T-cell activator beads (Stem Cell Technologies, San Diego, Calif.).

### Cell Cytotoxicity Assays

Cytotoxicity of the Lym-2 CAR T-cells are determined using the lactate dehydrogenase (LDH) cytotoxicity kit (Thermo Scientific, Carlsbad, Calif.). Activated T-cells are collected and 1×10⁶ cells are transduced with the Lym-2 CAR lentiviral construct as described above. Cells are activated used the T-cell activator beads (Stem Cell Technologies, San Diego, Calif.) for two days prior to cytotoxicity assays. The optimal number of target cells will be determined as per the manufacturer's protocol. For the assays, the appropriate target cells will be plated in triplicate in a 96 well plate for 24 hours at 37° C. in a 37° C. humidified 5% CO₂ incubator, followed by addition of activated CAR T-cells in ratios of 20:1, 10:1, 5:1 and 1:1, and incubated for 24 as above. Cells will be lysed at 37° C. for 45 mins and centrifuged at 1,250 rpm for 5 minutes. The supernatants are transferred to a fresh 96 well plate, followed by the addition of the reaction mixture for 30 minutes. The reaction is stopped using the stop solution and the plate read at 450nm with an absorbance correction at 650 nm.

### In Vivo Tumor Regression Assay

Foxn1 null mice are injected with immortalized B lymphoma cell line, Raji, which expresses the Lym-2 antigen. Two ×10⁶ Raji cells with 1×10⁶ human fibroblasts in 200 ul of phosphate buffered saline (PBS) are injected into the left flank of the pre-irradiated (400 rads) BALB/c mice in insure a high take rate of tumor. T-cells are activated for 2 days with the αCD3/CD28 activator complex (Stem Cell Technologies, San Diego, Calif.). The activated T-cells are then transduced with Lym-2 CAR lentiviral particles, followed by activation with the αCD3/CD28 activator complex for an additional 2 days. The activated T-cells expressing the Lym-2 CAR (2.5×10⁶) are injected intravenously into the mice on day 7 after tumor inoculation. Tumor sizes are assessed 3×/week using Vernier calipers and the tumor volumes calculated.

### Detection of Lym-2 CAR Expression

Analysis of the Lym-2 CAR T-cells for expression of the Lym-1 CAR, showed 28% of the transduced T-cells positive for Lym-2 (FIG. 38 middle panel). In contrast, only 1% of the un-transduced T-cells used as a control were positive for CAR expression (FIG. 38 left panel). CD19 transduced T-cells were used as a positive control and showed 52% expression of the CD19 CAR (FIG. 38 right panel).

### Cytotoxicity for Lym-2 CAR T-Cells

The cytolytic activity of the Lym-2 CAR T-cells was determined using Raji, a B-cell lymphoma cell line. Raji expresses the Lym-2 antigen, as determined by FACS analysis. Lym-2 CAR T-cells were added to the Raji cells in ratios of 20:1, 10:1, 5:1 and 1:1 of effector to target cells. Lym-2 CAR T-cells show increased lysis of the target Raji cells at ratios of 5:1 and 10:1 with a lysis rate of 22%. In comparison, untransduced T-cells did not lyse Raji cells at any of the ratios tested.

### Example 14

### NK Cell Transduction

### NK-92MI Transduction

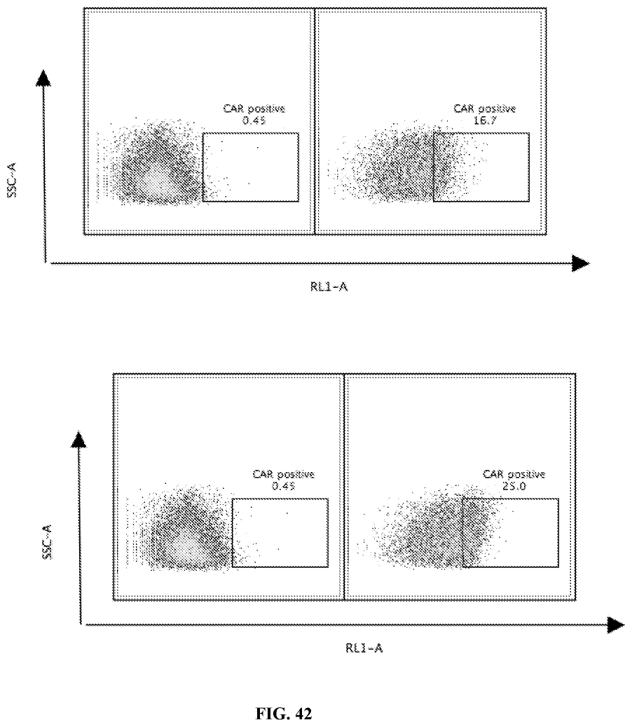

NK-92Mi cell line was purchased from ATCC (CRL-2408) and maintained in RPMI-1640 with 10% FBS. Before transduction, non-tissue treated 24-wells were incubated with 10 μg RetroNectin (Clontech T100A) in 300 μL Phosphate Buffered Saline (PBS) at room temperature for 2 hours. One million NK-92Mi cells and lentivirus (MOI=5) were mixed and added to the RetroNectin coated plates. The plates were then centrifuged at 28° C. 800g for 90 min. After centrifugation, the cells were maintained in a cell culture incubator overnight. After incubation, the cells were washed with PBS three times the following morning and the transduced NK-92Mi cells were then transferred to 24 well G-Rex (Wilson Wolf) plates for expansion. Seven days after Lentivirus transduction, the cells were washed 3× in wash buffer (4%BSA in PBS), stained with Biotein-Protein L (1 ug/1 million cells. Genscript) at 4° C. for 45 min, and washed 3× with wash buffer before adding 2 ul Streptavidin-APC (BD science) at 4° C. for 45 min. After a final 3 washes in wash buffer, the cells were analyzed by FACs (Attune) (FIG. 42).

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A chimeric antigen receptor (CAR) comprising: (a) an antigen binding domain of an anti-luteinizing hormone receptor (“LHR”) antibody, an anti-B7-H4 antibody, an anti-HLA-G, or an HLA-DR antibody (b) a CD8 α hinge domain; (c) a CD8 α transmembrane domain; (d) two or more costimulatory signaling regions; and (e) a CD3 zeta signaling domain.

2. The CAR of claim 1, wherein the two or more costimulatory signaling regions are selected from CD27, CD28, 4-IBB (CD 137), OX40, CD30, CD40, PD-1, ICOS, lymphocyte function-associated antigen-1 (LFA-1), CD2, CD7, CD27, LIGHT, NKG2C, and B7-H3.

3. The CAR of claim 1, wherein the antigen binding domain of the anti-LHR antibody, anti-B7-H4 antibody, an anti-HLA-G, or an HLA-DR antibody comprises an anti-LHR heavy chain (HC) variable region and an anti-LHR light chain (LC) variable region.

4. The CAR of claim 3, further comprising a linker polypeptide located between the anti-LHR HC, anti-B7-H4 HC, anti-HLA-G HC, or anti-HLA-DR HC variable region and the anti-LHR LC, anti-B7-H4 LC, anti-HLA-G LC or anti-HLA-DR LC variable region.

5. The CAR of claim 1, wherein the anti-LHR antibody HC comprises:
(a) a CDR1 comprising the amino acid sequence of GYSITSGYG, GFSLTTYG, or GYSFTGYY, or an equivalent of each thereof; and/or
(b) a CDR2 comprising the amino acid sequence of IHYSGST, IWGDGST, or IYPYNGVS, or an equivalent of each thereof; and/or
(c) a CDR3 comprising the amino acid sequence of ARSLRY, AEGSSLFAY, or ARERGLYQLRAMDY, or an equivalent of each thereof; and/or
the anti-LHR antibody LC comprises:
(a) a CDR1 comprising the amino acid sequence of SSVNY, QSLLNSGNQKNY, or QSISNN, or an equivalent of each thereof; and/or
(b) a CDR2 comprising the amino acid sequence of DTS, WAS, or NAS, or an equivalent of each thereof; and/or
(c) a CDR3 comprising the amino acid sequence of HQWSSYPYT, QNDYSYPLT, or QQSNSWPYT, or an equivalent of each thereof,
the anti-B7-H4 antibody HC comprises:
(a) a CDR1 comprising the amino acid sequence of GXTF GFTFSSFG, GFTFSSYG, or GYTFTDY, or an equivalent of each thereof; and/or
(b) a CDR2 comprising the amino acid sequence of ISSXXXT, INPNNGGT, ISSGSSTL, or ISSSNSTI, or an equivalent of each thereof; and/or
(c) a CDR3 comprising the amino acid sequence of ARPXYY, ARPLYYYGSVMDY, or ARPYYYGSSYDY or an equivalent of each thereof; and/or
the anti-B7-H4 antibody LC comprises:
(a) a CDR1 comprising the amino acid sequence of QSIVHXNGTY, ENIGSY, QSIVHRNGNTY, or QSIVHSNGNTY or an equivalent of each thereof; and/or
(b) a CDR2 comprising the amino acid sequence of KVS or AAT, or an equivalent of each thereof; and/or
(c) a CDR3 comprising the amino acid sequence of FQGSXVPXT, QHYYSTLVT, FQGSYVPPT, or FQGSHVPLT or an equivalent of each thereof,
the anti-HLA-G antibody HC comprises:
(a) a CDR1 comprising the amino acid sequence of GFNIKDTY or GFTFNTYA, or an equivalent of each thereof; and/or
(b) a CDR2 comprising the amino acid sequence of IDPANGNT or IRSKSNNYAT, or an equivalent of each thereof; and/or
(c) a CDR3 comprising the amino acid sequence of ARSYYGGFAY, or VRGGYWSFDV, or an equivalent of each thereof; and/or
the anti-HLA-G LC comprises:
(a) a CDR1 comprising the amino acid sequence of KSVSTSGYSY or KSLLHSNGNTY, or an equivalent of each thereof; and/or
(b) a CDR2 comprising the amino acid sequence of LVS or RMS, or an equivalent of each thereof; and/or
(c) a CDR3 comprising the amino acid sequence of QHSRELPRT or MQHLEYPYT, or an equivalent of each thereof,
wherein the anti-HLA-DR HC comprises:
(a) a CDR1 comprising the amino acid sequence of a CDRH1 of a Lym-1 antibody or a CDRH1 of a Lym-2 antibody, or an equivalent of each thereof; and/or
(b) a CDR2 comprising the amino acid sequence of a CDRH2 of a Lym-1 antibody or a CDRH2 of a Lym-2 antibody, or an equivalent of each thereof; and/or
(c) a CDR3 comprising the amino acid sequence of a CDRH3 of a Lym-1 antibody or a CDRH3 of a Lym-2 antibody, or an equivalent of each thereof; and/or
the anti-HLA-DR LC comprises:
(a) a CDR1 comprising the amino acid sequence of (i) a CDRL1 of a Lym-1 antibody or a CDRL1 of a Lym-2 antibody, or an equivalent of each thereof; and/or
(b) a CDR2 comprising the amino acid sequence of a CDRL2 of a Lym-1 antibody or a CDRL2 of a Lym-2 antibody, or an equivalent of each thereof; and/or
(c) a CDR3 comprising the amino acid sequence of a CDRL3 of a Lym-1 antibody or a CDRL3 of a Lym-2 antibody, or an equivalent of each thereof.

6.-11. (canceled)

12. The CAR of claim 5, wherein an equivalent comprises a polypeptide having at least 80% amino acid identity to polypeptide or a polypeptide that is encoded by a polynucleotide that hybridizes under conditions of high stringency to the complement of a polynucleotide encoding the polypeptide.

13. (canceled)

14. (canceled)

15. An isolated nucleic acid sequence encoding the CAR of claim 1.

16. (canceled)

17. The isolated nucleic acid sequence of claim 15, further comprising a Kozak consensus sequence located upstream of the antigen binding domain of the anti-LHR antibody, anti-B7-H4 antibody, anti-HLA-G antibody, anti-HLA-DR antibody, or an enhancer.

18. The isolated nucleic acid sequence of claim 15, further comprising an antibiotic resistance polynucleotide.

19. The isolated nucleic acid sequence of claim 15, further comprising a switch mechanism for controlling expression and/or activation of the CAR.

20.-23. (canceled)

24. An isolated cell comprising the CAR of claim 1.

25. The isolated cell of claim 24, wherein the isolated cell is an immune cell, that is optionally a T-cell or a natural killer (NK) cell.

26. (canceled)

27. A composition comprising a carrier and the CAR of claim 1.

28. The composition of claim 27, further comprising an antigen binding fragment capable of binding a peptide, wherein the peptide comprises an LHR protein or a fragment thereof, a B7-H4 protein or a fragment thereof, an HLA-G protein or a fragment thereof, or an HLA-DR protein or a fragment thereof.

29.-32. (canceled)

33. A method of producing anti-LHR CAR, anti-B7-H4 CAR, anti-HLA-G CAR, or anti-HLA-DR CAR expressing cells comprising:
(i) introducing a population of immune cells with a nucleic acid sequence encoding the CAR of claim 1; and
(ii) selecting a subpopulation of immune cells that have been successfully transduced with said nucleic acid sequence of step (i) thereby producing anti-LHR CAR, anti-B7-H4 CAR, anti-HLA-G CAR, or anti-HLA-DR CAR expressing cells.

34. The method of claim 33, wherein the immune cells are T-cells or a natural killer (NK) cells.

35. The method of claim 34, wherein the population of T-cells have been modified to reduce or eliminate expression of endogenous T-cell receptors.

36. The method of claim 35, wherein the population of T-cells were modified using a method that employs RNA interference or CRISPR.

37. A method of inhibiting the growth of a tumor and/or treating a cancer in a subject in need thereof, comprising administering to the subject an effective amount of the anti-LHR CAR, anti-B7-H4 CAR, anti-HLA-G CAR or anti-HLA-DR CAR expressing cells of claim 25.

38. The method of claim 37, wherein the anti-LHR CAR, anti-B7-H4 CAR, anti-HLA-G CAR or anti-HLA-DR CAR expressing cells are autologous or allogenic to the subject being treated.

39. The method of claim 37, wherein the tumor or cancer expresses or overexpresses LHR, B7-H4, HLA-G, or HLA-DR.

40. The method of claim 37, wherein the tumor is a solid tumor, optionally an ovarian tumor or a prostate cancer tumor and/or the cancer is and ovarian cancer or a prostate cancer.

41. The method of claim 37, wherein the subject is a human, an animal, a non-human primate, a dog, cat, a sheep, a mouse, a horse, or a cow.

42.-157. (canceled)


SEQUENCE LISTING (138 sequences)

SEQ ID NO: 1 - protein, 211 aa, Homo sapiens
IGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKA

SEQ ID NO: 2 - protein, 315 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWHLAGYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 3 - DNA, 48 bp, Artificial Sequence
  Description of Artificial Sequence Synthetic oligonucleotide
ctcgagcccaaatcttgtgacaaaactcacacatgcccaccgtgcccg

SEQ ID NO: 4 - DNA, 81 bp, Unknown
  Description of Unknown Sequence CD28 transmembrane region sequence
ttttgggtgctggtggtggttggtggagtcctggcttgctatagcttgctagtaacagtggcctttattattttctgggtg

SEQ ID NO: 5 - DNA, 126 bp, Unknown
  Description of Unknown Sequence 4-1BB co-stimulatory signaling region sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 6 - DNA, 123 bp, Unknown
  Description of Unknown Sequence CD28 co-stimulatory signaling region sequence
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 7 - DNA, 339 bp, Unknown
  Description of Unknown Sequence CD3 zeta signaling region sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 8 - protein, 237 aa, Homo sapiens
GDTRPRFLEEVKFECHFFNGTERVRLLERRVHNQEEYARYDSDVGEYRAVTELGRPDAEYWNSQKDLLERRRAAVDTYCRHNYGVGESFTVQRRVQPKVTVYPSKTQPLQHHNLLVCSVNGFYPGSIEVRWFRNGQEEKTGVVSTGLIQNGDWTFQTLVMLETVPQSGEVYTCQVEHPSVMSPLTVEWRARSESAQSKMLSGVGGFVLGLLFLGAGLFIYFRNQKGHSGLPPTGFLS

SEQ ID NO: 9 - protein, 237 aa, Homo sapiens
GDTRPRFLELLKSECHFFNGTERVRFLERHFHNQEEYARFDSDVGEYRAVFELGRPDAEYWNSQKDLLEQKRGQVDNYCRHNYGVVESFTVQRRVHPQVTVYPAKTQPLQHHNLLVCSVSGFYPGSIEVRWFRNGQEEKAGVVSTGLIQNGDWTFQTLVMLETFPRSGEVYTCQVEHPSVTSPLTVEWSARSESAQSKMLSGVGGFVLGLLFLGAGLFIYFRNQKGHSGLQPTGFLS

SEQ ID NO: 10 - protein, 237 aa, Homo sapiens
GDTRPRFLEYSTSECHFFNGTERVRYLDRYFHNQEENVRFDSDVGEFRAVTELGRPDAEYWNSQKDLLEQKRGRVDNYCRHNYGVVESFTVQRRVHPKVTVYPSKTQPLQHHNLLVCSVSGFYPGSIEVRWFRNGQEEKTGVVSTGLIQNGDWTFQTLVMLETVPRSGEVYTCQVEHPSVTSPLTVEWRARSESAQSKMLSGVGGFVLGLLFLGAGLFIYFRNQKGHSGLQPRGFLS

SEQ ID NO: 11 - protein, 699 aa, Homo sapiens
MKQRFSALQLLKLLLLLQPPLPRALREALCPEPCNCVPDGALRCPGPTAGLTRLSLAYLPVKVIPSQAFRGLNEVIKIEISQIDSLERIEANAFDNLLNLSEILIQNTKNLRYIEPGAFINLPRLKYLSICNTGIRKFPDVTKVFSSESNFILEICDNLHITTIPGNAFQGMNNESVTLKLYGNGFEEVQSHAFNGTTLTSLELKENVHLEKMHNGAFRGATGPKTLDISSTKLQALPSYGLESIQRLIATSSYSLKKLPSRETFVNLLEATLTYPSHCCAFRNLPTKEQNFSHSISENFSKQCESTVRKVNNKTLYSSMLAESELSGWDYEYGFCLPKTPRCAPEPDAFNPCEDIMGYDFLRVLIWLINILAIMGNMTVLFVLLTSRYKLTVPRFLMCNLSFADFCMGLYLLLIASVDSQTKGQYYNHAIDWQTGSGCSTAGFFTVFASELSVYTLTVITLERWHTITYAIHLDQKLRLRHAILIMLGGWLFSSLIAMLPLVGVSNYMKVSICFPMDVETTLSQVYILTILILNVVAFFIICACYIKIYFAVRNPELMATNKDTKIAKKMAILIFTDFTCMAPISFFAISAAFKVPLITVTNSKVLLVLFYPINSCANPFLYAIFTKTFQRDFFLLLSKFGCCKRRAELYRRKDFSAYTSNCKNGFTGSNKPSQSTLKLSTLHCQGTALLDKTRYTEC

SEQ ID NO: 12 - protein, 700 aa, Mus musculus
MGRRVPALRQLLVLAMLVLKQSQLHSPELSGSRCPEPCDCAPDGALRCPGPRAGLARLSLTYLPVKVIPSQAFRGLNEVVKIEISQSDSLERIEANAFDNLLNLSEILIQNTKNLLYIEPGAFTNLPRLKYLSICNTGIRTLPDVSKISSSEFNFILEICDNLYITTIPGNAFQGMNNESITLKLYGNGFEEVQSHAFNGTTLISLELKENIYLEKMHSGTFQGATGPSILDVSSTKLQALPSHGLESIQTLIATSSYSLKTLPSREKFTSLLVATLTYPSHCCAFRNLPKKEQNFSFSIFENFSKQCESTVREANNETLYSAIFEENELSGWDYDYDFCSPKTLQCTPEPDAFNPCEDIMGYAFLRVLIWLINILAIFGNLTVLFVLLTSRYKLTVPRFLMCNLSFADFCMGLYLLLIASVDSQTKGQYYNHAIDWQTGSGCSAAGFFTVFASELSVYTLTVITLERWHTITYAVQLDQKLRLRHAIPIMLGGWIFSTLMATLPLVGVSSYMKVSICLPMDVESTLSQVYILSILLLNAVAFVVICACYVRIYFAVQNPELTAPNKDTKIAKKMAILIFTDFTCMAPISFFAISAAFKVPLITVTNSKVLLVLFYPVNSCANPFLYAVFTKAFQRDFFLLLSRFGCCKHRAELYRRKEFSACTFNSKNGFPRSSKPSQAALKLSIVHCQQPTPPRVLIQ

SEQ ID NO: 13 - protein, 700 aa, Rattus norvegicus
MGRRVPALRQLLVLAVLLLKPSQLQSRELSGSRCPEPCDCAPDGALRCPGPRAGLARLSLTYLPVKVIPSQAFRGLNEVVKIEISQSDSLERIEANAFDNLLNLSELLIQNTKNLLYIEPGAFTNLPRLKYLSICNTGIRTLPDVTKISSSEFNFILEICDNLHITTIPGNAFQGMNNESVTLKLYGNGFEEVQSHAFNGTTLISLELKENIYLEKMHSGAFQGATGPSILDISSTKLQALPSHGLESIQTLIALSSYSLKTLPSKEKFTSLLVATLTYPSHCCAFRNLPKKEQNFSFSIFENFSKQCESTVRKADNETLYSAIFEENELSGWDYDYGFCSPKTLQCAPEPDAFNPCEDIMGYAFLRVLIWLINILAIFGNLTVLFVLLTSRYKLTVPRFLMCNLSFADFCMGLYLLLIASVDSQTKGQYYNHAIDWQTGSGCGAAGFFTVFASELSVYTLTVITLERWHTITYAVQLDQKLRLRHAIPIMLGGWLFSTLIATMPLVGISNYMKVSICLPMDVESTLSQVYILSILILNVVAFVVICACYIRIYFAVQNPELTAPNKDTKIAKKMAILIFTDFTCMAPISFFAISAAFKVPLITVTNSKILLVLFYPVNSCANPFLYAIFTKAFQRDFLLLLSRFGCCKRRAELYRRKEFSAYTSNCKNGFPGASKPSQATLKLSTVHCQQPIPPRALTH

SEQ ID NO: 14 - protein, 8 aa, Artificial Sequence
  Description of Artificial Sequence Synthetic peptide
GFTFSSFG

SEQ ID NO: 15 - protein, 8 aa, Artificial Sequence
  Description of Artificial Sequence Synthetic peptide
GFTFSSYG

SEQ ID NO: 16 - protein, 7 aa, Artificial Sequence
  Description of Artificial Sequence Synthetic peptide
GYTFTDY

SEQ ID NO: 17 - protein, 8 aa, Artificial Sequence
  Description of Artificial Sequence Synthetic peptide
ISSGSSTL

SEQ ID NO: 18 - protein, 8 aa, Artificial Sequence
  Description of Artificial Sequence Synthetic peptide
ISSSNSTI

SEQ ID NO: 19 - protein, 8 aa, Artificial Sequence
  Description of Artificial Sequence Synthetic peptide
INPNNGGT

SEQ ID NO: 20 - protein, 13 aa, Artificial Sequence
  Description of Artificial Sequence Synthetic peptide
ARPLYYYGSVMDY

SEQ ID NO: 21 - protein, 11 aa, Artificial Sequence
  Description of Artificial Sequence Synthetic peptide
RPYYYGSSYDY

SEQ ID NO: 22 - protein, 11 aa, Artificial Sequence
  Description of Artificial Sequence Synthetic peptide
QSIVHRNGNTY

SEQ ID NO: 23 - protein, 11 aa, Artificial Sequence
  Description of Artificial Sequence Synthetic peptide
QSIVHSNGNTY

SEQ ID NO: 24 - protein, 6 aa, Artificial Sequence
  Description of Artificial Sequence Synthetic peptide
ENIGSY

SEQ ID NO: 25 - protein, 9 aa, Artificial Sequence
  Description of Artificial Sequence Synthetic peptide
FQGSYVPPT

SEQ ID NO: 26 - protein, 9 aa, Artificial Sequence
  Description of Artificial Sequence Synthetic peptide
FQGSHVPLT

SEQ ID NO: 27 - protein, 9 aa, Artificial Sequence
  Description of Artificial Sequence Synthetic peptide
QHYYSTLVT

SEQ ID NO: 28 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 29 - protein, 51 aa, Homo sapiens
PAKPTTTPAPRPPTPAPTIASQPLSLRPEACRPAAGGAVHTRGLDFACDIY

SEQ ID NO: 30 - protein, 49 aa, Mus musculus
KVNSTTTKPVLRTPSPVHPTGTSQPQRPEDCRPRGSVKGTGLDFACDIY

SEQ ID NO: 31 - protein, 51 aa, Felis catus
PVKPTTTPAPRPPTQAPITTSQRVSLRPGTCQPSAGSTVEASGLDLSCDIY

SEQ ID NO: 32 - protein, 21 aa, Homo sapiens
IYIWAPLAGTCGVLLLSLVIT

SEQ ID NO: 33 - protein, 21 aa, Mus musculus
IWAPLAGICVALLLSLIITLI

SEQ ID NO: 34 - protein, 21 aa, Rattus norvegicus
IWAPLAGICAVLLLSLVITLI

SEQ ID NO: 35 - protein, 42 aa, Unknown
  Description of Unknown 4-1BB costimulatory signaling region sequence
KRGRKKLLYIFKQPFMRPVQTTQEEDGCSCRFPEEEEGGCEL

SEQ ID NO: 36 - protein, 220 aa, Unknown
  Description of Unknown CD28 sequence
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 37 - DNA, 105 bp, Unknown
  Description of Unknown ICOS costimulatory signaling region sequence
acaaaaaagaagtattcatccagtgtgcacgaccctaacggtgaatacatgttcatgagagcagtgaacacagccaaaaaatccagactcacagatgtgacccta

SEQ ID NO: 38 - DNA, 108 bp, Unknown
  Description of Unknown OX40 costimulatory signaling region sequence
agggaccagaggctgccccccgatgcccacaagccccctgggggaggcagtttccggacccccatccaagaggagcaggccgacgcccactccaccctggccaagatc

SEQ ID NO: 39 - protein, 112 aa, Unknown
  Description of Unknown CD3 zeta signaling domain sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 15 aa, Artificial Sequence
  Description of Artificial Sequence Synthetic peptide
GGGGSGGGGSGGGGS

SEQ ID NO: 41 - protein, 5 aa, Artificial Sequence
  Description of Artificial Sequence Synthetic peptide
GGGGS

SEQ ID NO: 42 - protein, 14 aa, Artificial Sequence
  Description of Artificial Sequence Synthetic peptide
GGGGSGGGGSGGGG

SEQ ID NO: 43 - protein, 13 aa, Artificial Sequence
  Description of Artificial Sequence Synthetic peptide
EFGAGLVLGGQFM

SEQ ID NO: 44 - protein, 9 aa, Artificial Sequence
  Description of Artificial Sequence Synthetic peptide
GYSITSGYG

SEQ ID NO: 45 - protein, 7 aa, Artificial Sequence
  Description of Artificial Sequence Synthetic peptide
IHYSGST

SEQ ID NO: 46 - protein, 6 aa, Artificial Sequence
  Description of Artificial Sequence Synthetic peptide
ARSLRY

SEQ ID NO: 47 - protein, 5 aa, Artificial Sequence
  Description of Artificial Sequence Synthetic peptide
SSVNY

SEQ ID NO: 48 - protein, 9 aa, Artificial Sequence
  Description of Artificial Sequence Synthetic peptide
HQWSSYPYT

SEQ ID NO: 49 - protein, 8 aa, Artificial Sequence
  Description of Artificial Sequence Synthetic peptide
GFSLTTYG

SEQ ID NO: 50 - protein, 7 aa, Artificial Sequence
  Description of Artificial Sequence Synthetic peptide
IWGDGST

SEQ ID NO: 51 - protein, 9 aa, Artificial Sequence
  Description of Artificial Sequence Synthetic peptide
AEGSSLFAY

SEQ ID NO: 52 - protein, 12 aa, Artificial Sequence
  Description of Artificial Sequence Synthetic peptide
QSLLNSGNQKNY

SEQ ID NO: 53 - protein, 9 aa, Artificial Sequence
  Description of Artificial Sequence Synthetic peptide
QNDYSYPLT

SEQ ID NO: 54 - protein, 8 aa, Artificial Sequence
  Description of Artificial Sequence Synthetic peptide
GYSFTGYY

SEQ ID NO: 55 - protein, 8 aa, Artificial Sequence
  Description of Artificial Sequence Synthetic peptide
IYPYNGVS

SEQ ID NO: 56 - protein, 14 aa, Artificial Sequence
  Description of Artificial Sequence Synthetic peptide
ARERGLYQLRAMDY

SEQ ID NO: 57 - protein, 6 aa, Artificial Sequence
  Description of Artificial Sequence Synthetic peptide
QSISNN

SEQ ID NO: 58 - protein, 9 aa, Artificial Sequence
  Description of Artificial Sequence Synthetic peptide
QQSNSWPYT

SEQ ID NO: 59 - protein, 7 aa, Artificial Sequence
  Description of Artificial Sequence Synthetic peptide
  MOD_RES (4)..(6): Any amino acid
ISSXXXT

SEQ ID NO: 60 - protein, 6 aa, Artificial Sequence
  Description of Artificial Sequence Synthetic peptide
  MOD_RES (4)..(4): Any amino acid
ARPXYY

SEQ ID NO: 61 - protein, 10 aa, Artificial Sequence
  Description of Artificial Sequence Synthetic peptide
  MOD_RES (6)..(6): Any amino acid
QSIVHXNGTY

SEQ ID NO: 62 - protein, 9 aa, Artificial Sequence
  Description of Artificial Sequence Synthetic peptide
  MOD_RES (5)..(5): Any amino acid
  MOD_RES (8)..(8): Any amino acid
FQGSXVPXT

SEQ ID NO: 63 - protein, 12 aa, Artificial Sequence
  Description of Artificial Sequence Synthetic peptide
ARPYYYGSSYDY

SEQ ID NO: 64 - protein, 8 aa, Artificial Sequence
  Description of Artificial Sequence Synthetic peptide
GFNIKDTY

SEQ ID NO: 65 - protein, 8 aa, Artificial Sequence
  Description of Artificial Sequence Synthetic peptide
GFTFNTYA

SEQ ID NO: 66 - protein, 8 aa, Artificial Sequence
  Description of Artificial Sequence Synthetic peptide
IDPANGNT

SEQ ID NO: 67 - protein, 10 aa, Artificial Sequence
  Description of Artificial Sequence Synthetic peptide
IRSKSNNYAT

SEQ ID NO: 68 - protein, 10 aa, Artificial Sequence
  Description of Artificial Sequence Synthetic peptide
ARSYYGGFAY

SEQ ID NO: 69 - protein, 10 aa, Artificial Sequence
  Description of Artificial Sequence Synthetic peptide
VRGGYWSFDV

SEQ ID NO: 70 - protein, 10 aa, Artificial Sequence
  Description of Artificial Sequence Synthetic peptide
KSVSTSGYSY

SEQ ID NO: 71 - protein, 11 aa, Artificial Sequence
  Description of Artificial Sequence Synthetic peptide
KSLLHSNGNTY

SEQ ID NO: 72 - protein, 9 aa, Artificial Sequence
  Description of Artificial Sequence Synthetic peptide
QHSRELPRT

SEQ ID NO: 73 - protein, 9 aa, Artificial Sequence
  Description of Artificial Sequence Synthetic peptide
MQHLEYPYT

SEQ ID NO: 74 - protein, 84 aa, Unknown
  Description of Unknown LHR protein sequence
REALCPEPCNCVPDGALRCPGPTAGLTRLSLAYLPVKVIPSQAFRGLNEVIKIEISQIDSLERIEANAFDNLLNLSEILIQNTK

SEQ ID NO: 75 - protein, 255 aa, Unknown
  Description of Unknown LHR protein sequence
RALREALCPEPCNCVPDGALRCPGPTAGLTRLSLAYLPVKVIPSQAFRGLNEVIKIEISQIDSLERIEANAFDNLLNLSEILIQNTKNLRYIEPGAFINLPRLKYLSICNTGIRKFPDVTKVFSSESNFILEICDNLHITTIPGNAFQGMNNESVTLKLYGNGFEEVQSHAFNGTTLTSLELKENVHLEKMHNGAFRGATGPKTLDISSTKLQALPSYGLESIQRLIATSSYSLKKLPSRETFVNLLEATLTYPS

SEQ ID NO: 76 - DNA, 309 bp, Artificial Sequence
  Description of Artificial Sequence Synthetic polynucleotide
gaggtgcagctggaggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtaccctccactatgcagacacagtgaagggccgattcaccatctccagagacaatcccaagaacaccctgttcctgcaaatgaaactaccctcactatgctatggactactggggtcaaggaacctcagtcaccgtctcctc

SEQ ID NO: 77 - protein, 103 aa, Artificial Sequence
  Description of Artificial Sequence Synthetic polypeptide
EVQLEESGGGLVQPGGSRKLSCAASGFTFSSFGMHWVRQAPEKGLEWVAYISSGSSTLHYADTVKGRFTISRDNPKNTLFLQMKLPSLCYGLLGSRNLSHRLL

SEQ ID NO: 78 - DNA, 360 bp, Artificial Sequence
  Description of Artificial Sequence Synthetic polynucleotide
gatgtgcagctggtggagtctgggggaggtttagtgcagcctggagggtcccggaaactctcctgtgcagcctctggattcactttcagtagctatggaattcactgggttcgtcaggttccagagaaggggctggagtgggtcgcatttattagtagtagcaattctaccatctactatgcagacacagtgaagggccgattcaccatctccagagacaatgccgagaacaccctgttcctgcaaatgaccagtctaaggtctgaggacacggccatgtattactgtgcaagacccctttactactatggtagcgttatggactactggggtcaaggaacctctgtcaccgtctcctca

SEQ ID NO: 79 - protein, 120 aa, Artificial Sequence
  Description of Artificial Sequence Synthetic polypeptide
DVQLVESGGGLVQPGGSRKLSCAASGFTFSSYGIHWVRQVPEKGLEWVAFISSSNSTIYYADTVKGRFTISRDNAENTLFLQMTSLRSEDTAMYYCARPLYYYGSVMDYWGQGTSVTVSS

SEQ ID NO: 80 - DNA, 357 bp, Artificial Sequence
  Description of Artificial Sequence Synthetic polynucleotide
gaggtccagctgcaacaatctggacctgagctggtgaagcctggggcttcagtgaagatatcctgtaaggcttctggatacacgttcactgactactacatgaactggatgaagcagagccatggaaagagtcttgagtggattggagatattaatcctaacaatggtggtactagctacaaccagaagttcaagggcaaggccacattgactgtagacaagtcctccagcacagcctacatggaactccgcagcctgacatctgaggactctgcagtctattactgtgcaagaccttattactacggtagtagctacgactactggggccaaggcaccactctcacagtctcctca

SEQ ID NO: 81 - protein, 118 aa, Artificial Sequence
  Description of Artificial Sequence Synthetic polypeptide
EVQLQQSGPELVKPGASVKISCKASGYTFTDYYMNWMKQSHGKSLEWIGDINPNNGGTSYNQKFKGKATLTVDKSSSTAYMELRSLTSEDSAVYYCARPYYYGSSYDYWGQGTTLTVS

SEQ ID NO: 82 - DNA, 336 bp, Artificial Sequence
  Description of Artificial Sequence Synthetic polynucleotide
gacattgtgatcacccaaactccactctccctgcctgtcagtcttggagatcaagcctccatctcttgcagatctagtcagagcattgtacataggaatggaaacacctatttagaatggtacttgcagcaaccaggccagtctccaaagctcctgatctacaaagtttccaaccgattttctggggtcccagacaggttcagtggcagtggatcagggacagatttcacactcaagatcagcagagtggaggctgaagatctgggagtttattactgctttcaaggttcatatgttcctccgacgttcggtggaggcaccaagctggaaatcaaa

SEQ ID NO: 83 - protein, 112 aa, Artificial Sequence
  Description of Artificial Sequence Synthetic polypeptide
DIVITQTPLSLPVSLGDQASISCRSSQSIVHRNGNTYLEWYLQQPGQSPKLLIYKVSNRFSGVPDRFSGSGSGTDFTLKISRVEAEDLGVYYCFQGSYVPPTFGGGTKLEIK

SEQ ID NO: 84 - DNA, 336 bp, Artificial Sequence
  Description of Artificial Sequence Synthetic polynucleotide
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagataagtagagtggaggctgaggatctgggagtttattactgctttcaaggttcacatgttcctctcacgttcggtgcagggaccaagctggaactgaaa

SEQ ID NO: 85 - protein, 112 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 86 - DNA, 321 bp, Artificial Sequence
  Description of Artificial Sequence Synthetic polynucleotide
gacatccagatgactcagtctccagcttccctgtctgcatctgtgggagaaactgtcaccatcacatgtcgagcaagtgaaaatattggcagttatttagcatggtatcagcagaaacagggaaaatctcctcagctcctggtctatgctgcaacactcttagcagatggtgtgccatcaaggttcagtggcagtggatcaggcacacagttttctctcaagatcaacagcctgcagtctgaagatgttgcgagatattactgtcaacattattatagtactctggtcacgttcggtgctgggaccaagctggaactgaaa

SEQ ID NO: 87 - protein, 107 aa, Artificial Sequence
  Description of Artificial Sequence Synthetic polypeptide
DIQMTQSPASLSASVGETVTITCRASENIGSYLAWYQQKQGKSPQLLVYAATLLADGVPSRFSGSGSGTQFSLKINSLQSEDVARYYCQHYYSTLVTFGAGTKLELK

SEQ ID NO: 88 - DNA, 351 bp, Artificial Sequence
  Description of Artificial Sequence Synthetic polynucleotide
caggtgcagctgcaggagtcaggggcagagcttgtgaagccaggggcctcagtcaagttgtcctgcacagcttctggcttcaacattaaagacacctatatgcactgggtgaagcagaggcctgaacagggcctggagtggattggaaggattgatcctgcgaatggtaatactaaatatgacccgaagttccagggcaaggccactataacagcagacacatcctccaacacagcctacctgcagctcagcagcctgacatctgaggacactgccgtctattactgtgctaggagttactacggggggtttgcttactggggccaagggactctggtcactgtctctgca

SEQ ID NO: 89 - protein, 117 aa, Artificial Sequence
  Description of Artificial Sequence Synthetic polypeptide
QVQLQESGAELVKPGASVKLSCTASGFNIKDTYMHWVKQRPEQGLEWIGRIDPANGNTKYDPKFQGKATITADTSSNTAYLQLSSLTSEDTAVYYCARSYYGGFAYWGQGTLVTVSA

SEQ ID NO: 90 - DNA, 357 bp, Artificial Sequence
  Description of Artificial Sequence Synthetic polynucleotide
gaggtgcagctgcaggagtctggtggaggattggtgcagcctaaaggatcattgaaactctcatgtgccgcctttggtttcaccttcaatacctatgccatgcactgggtccgccaggctccaggaaagggtttggaatgggttgctcgcataagaagtaaaagtaataattatgcaacatattatgccgattcagtgaaagacagattcaccatctccagagatgattcacaaagcatgctctctctgcaaatgaacaacctgaaaactgaggacacagccatttattactgtgtgagagggggttactggagcttcgatgtctggggcgcagggaccacggtcaccgtctcctca

SEQ ID NO: 91 - protein, 119 aa, Artificial Sequence
  Description of Artificial Sequence Synthetic polypeptide
EVQLQESGGGLVQPKGSLKLSCAAFGFTFNTYAMHWVRQAPGKGLEWVARIRSKSNNYATYYADSVKDRFTISRDDSQSMLSLQMNNLKTEDTAIYYCVRGGYWSFDVWGAGTTVTVSS

SEQ ID NO: 92 - DNA, 333 bp, Artificial Sequence
  Description of Artificial Sequence Synthetic polynucleotide
gatattgtgctcacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgtatccaacctagaatctggggtccctgccaggttcagtggcagtgggtctgggacagacttcaccctcaacatccatcctgtggaggaggaggatgctgcaacctattactgtcagcacagtagggagcttcctcggacgttcggtggaggcaccaagctggaaatcaaa

SEQ ID NO: 93 - protein, 111 aa, Artificial Sequence
  Description of Artificial Sequence Synthetic polypeptide
DIVLTQSPASLAVSLGQRATISCRASKSVSTSGYSYMHWYQQKPGQPPKLLIYLVSNLESGVPARFSGSGSGTDFTLNIHPVEEEDAATYYCQHSRELPRTFGGGTKLEIK

SEQ ID NO: 94 - DNA, 336 bp, Artificial Sequence
  Description of Artificial Sequence Synthetic polynucleotide
gatattgtgatcacacagactacaccctctgtacctgtcactcctggagagtcagtatccatctcctgtaggtctagtaagagtctcctgcatagtaatggcaacacttacttgtattggttcctgcagaggccaggccagtctcctcagctcctgatatctcggatgtccagccttgcctcaggagtcccagacaggttcagtggcagtgggtcaggaactgctttcacactgagaatcagtagagtggaggctgaggatgtgggtgtttattactgtatgcaacatctagaatatccgtatacgttcggaggggggaccaagctggaaataaaa

SEQ ID NO: 95 - protein, 112 aa, Artificial Sequence
  Description of Artificial Sequence Synthetic polypeptide
DIVITQTTPSVPVTPGESVSISCRSSKSLLHSNGNTYLYWFLQRPGQSPQLLISRMSSLASGVPDRFSGSGSGTAFTLRISRVEAEDVGVYYCMQHLEYPYTFGGGTKLEIK

SEQ ID NO: 96 - protein, 8 aa, Artificial Sequence
  Description of Artificial Sequence Synthetic peptide
GFSLTSYG

SEQ ID NO: 97 - protein, 8 aa, Artificial Sequence
  Description of Artificial Sequence Synthetic peptide
GFTFSNYW

SEQ ID NO: 98 - protein, 7 aa, Artificial Sequence
  Description of Artificial Sequence Synthetic peptide
IWSDGST

SEQ ID NO: 99 - protein, 10 aa, Artificial Sequence
  Description of Artificial Sequence Synthetic peptide
IRFKSHNYAT

SEQ ID NO: 100 - protein, 12 aa, Artificial Sequence
  Description of Artificial Sequence Synthetic peptide
ASHYGSTLAFAS

SEQ ID NO: 101 - protein, 16 aa, Artificial Sequence
  Description of Artificial Sequence Synthetic peptide
TRRIGNSDYDWWYFDV

SEQ ID NO: 102 - DNA, 354 bp, Artificial Sequence
  Description of Artificial Sequence Synthetic polynucleotide
caggtgcagctgaaggagtcaggacctggcctggtggcgccctcacagagcctgtccatcacatgcaccatctcagggttctcattaaccagctatggtgtacactgggttcgccagcctccaggaaagggtctggagtggctggtagtgatatggagtgatggaagcacaacctataattcagctctcaaatccagactgagcatcagcaaggacaactccaagagccaagttttcttaaaaatgaacagtctccaaactgatgacacagccatatactactgtgccagtcactacggtagtacccttgcctttgcttcctggggccacgggactctggtcactgtctctgca

SEQ ID NO: 103 - protein, 116 aa, Artificial Sequence
  Description of Artificial Sequence Synthetic polypeptide
QLKESGPGLVAPSQSLSITCTISGFSLTSYGVHWVRQPPGKGLEWLVVIWSDGSTTYNSALKSRLSISKDNSKSQVFLKMNSLQTDDTAIYYCASHYGSTLAFASWGHGTLVTVSA

SEQ ID NO: 104 - DNA, 381 bp, Artificial Sequence
  Description of Artificial Sequence Synthetic polynucleotide
gaagtgcagcttgaggagtctggaggaggcttggtgcaacctggaggctccatgaaactctcctgtgttgcctctggattcactttcagtaactattggatgaactgggtccgccagtctccagagaaggggcttgagtgggttgctgaaattagatttaaatctcataattatgcaacacattttgcggagtctgtgaaagggaggttcaccatctcaagagatgattccaaaagtagtgtctacctgcaaatgaacaacttaagagctgaagacactggcatttattactgtaccaggaggataggaaactctgattacgactggtggtacttcgatgtctggggcgcagggacctcagtcaccgtctcctcagctagc

SEQ ID NO: 105 - protein, 127 aa, Artificial Sequence
  Description of Artificial Sequence Synthetic polypeptide
EVQLEESGGGLVQPGGSMKLSCVASGFTFSNYWMNWVRQSPEKGLEWVAEIRFKSHNYATHFAESVKGRFTISRDDSKSSVYLQMNNLRAEDTGIYYCTRRIGNSDYDWWYFDVWGAGTSVTVSSAS

SEQ ID NO: 106 - protein, 6 aa, Artificial Sequence
  Description of Artificial Sequence Synthetic peptide
VNIYSY

SEQ ID NO: 107 - protein, 6 aa, Artificial Sequence
  Description of Artificial Sequence Synthetic peptide
QNVGNN

SEQ ID NO: 108 - protein, 8 aa, Artificial Sequence
  Description of Artificial Sequence Synthetic peptide
QHHYGTFT

SEQ ID NO: 109 - protein, 9 aa, Artificial Sequence
  Description of Artificial Sequence Synthetic peptide
QQYNTYPFT

SEQ ID NO: 110 - DNA, 318 bp, Artificial Sequence
  Description of Artificial Sequence Synthetic polynucleotide
gacatccagatgactcagtctccagcctccctatctgcatctgtgggagaaactgtcaccatcatatgtcgagcaagtgtgaatatttacagttatttagcatggtatcagcagaaacagggaaaatctcctcagctcctggtctataatgccaaaatcttagcagaaggtgtgccatcaaggttcagtggcagtggatcaggcacacagttttctctgaagatcaacagcctgcagcctgaagattttgggagttattactgtcaacatcattatggtacattcacgttcggctcggggacaaagttggaaataaaa

SEQ ID NO: 111 - protein, 106 aa, Artificial Sequence
  Description of Artificial Sequence Synthetic polypeptide
DIQMTQSPASLSASVGETVTIICRASVNIYSYLAWYQQKQGKSPQLLVYNAKILAEGVPSRFSGSGSGTQFSLKINSLQPEDFGSYYCQHHYGTFTFGSGTKLEIK

SEQ ID NO: 112 - DNA, 321 bp, Artificial Sequence
  Description of Artificial Sequence Synthetic polynucleotide
gacattgtgatgacccagtctcacaaattcatgtccacatcagtaggagacagggtcagcgtcacctgcaaggccagtcagaatgtgggtaataatgtagcctggtatcaacagaaaccagggcaatctcctaaagtactgatttactcggcatcctaccggtacagtggagtccctgatcgcttcacaggcagtggatctgggacagatttcactctcaccatcagtaatgtgcagtctgaagacttggcagagtatttctgtcagcaatataacacctatccattcacgttcggctcggggacaaagttggaaataaaa

SEQ ID NO: 113 - protein, 107 aa, Artificial Sequence
  Description of Artificial Sequence Synthetic polypeptide
DIVMTQSHKFMSTSVGDRVSVTCKASQNVGNNVAWYQQKPGQSPKVLIYSASYRYSGVPDRFTGSGSGTDFTLTISNVQSEDLAEYFCQQYNTYPFTFGSGTKLEIK

SEQ ID NO: 114 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 115 - protein, 330 aa, Homo sapiens [sequence omitted]

SEQ ID NO: 116 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 117 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 118 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 119 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 120 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 121 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 122 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 24 bp, Artificial Sequence
  Description of Artificial Sequence Synthetic primer
cgcctgtgatatctacatctgggc

SEQ ID NO: 124 - DNA, 20 bp, Artificial Sequence
  Description of Artificial Sequence Synthetic primer
atcggcagctacagccatct

SEQ ID NO: 125 - DNA, 42 bp, Artificial Sequence
  Description of Artificial Sequence Synthetic primer
tcgatcaagcttgccgccaccatggcttccctggggcagatc

SEQ ID NO: 126 - DNA, 30 bp, Artificial Sequence
  Description of Artificial Sequence Synthetic primer
tgtgtgagttttgtcagcctttgacagctg

SEQ ID NO: 127 - DNA, 36 bp, Artificial Sequence
  Description of Artificial Sequence Synthetic primer
ctaaactcaaaggctgacaaaactcacacatgccca

SEQ ID NO: 128 - DNA, 42 bp, Artificial Sequence
  Description of Artificial Sequence Synthetic primer
tgattaatgatcaatgaattctcatttacccggagacaggga

SEQ ID NO: 129 - protein, 438 aa, Artificial Sequence
  Description of Artificial Sequence Synthetic polypeptide
IGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 710 aa, Artificial Sequence
  Description of Artificial Sequence Synthetic polypeptide
  MISC_FEATURE (1)..(374): Extracellular
  MISC_FEATURE (19)..(29): This region may or may not be present in its entirety
  MOD_RES (125)..(125): Ile or Phe
  MOD_RES (135)..(135): Arg or Gln
  MOD_RES (142)..(142): Cys or Arg
  MOD_RES (155)..(155): Val or Phe
  MOD_RES (205)..(205): Phe or Val
  MOD_RES (302)..(302): Asn or Ser
  MOD_RES (323)..(323): Asn or Ser
  MISC_FEATURE (328)..(335): This region may or may not be present in its entirety
  MOD_RES (354)..(354): Cys or Ser
  MOD_RES (365)..(365): Glu or Lys
  MISC_FEATURE (375)..(396): Transmembrane
  MOD_RES (379)..(379): Leu or Phe
  MOD_RES (384)..(384): Ala or Val
  MOD_RES (385)..(385): Ile or Thr
  MISC_FEATURE (397)..(406): Intracellular
  MOD_RES (403)..(403): Thr or Ile
  MISC_FEATURE (407)..(429): Transmembrane
  MOD_RES (409)..(409): Met or Thr
  MISC_FEATURE (430)..(450): Extracellular
  MISC_FEATURE (450)..(525): Disulfide bond
  MISC_FEATURE (451)..(473): Transmembrane
  MOD_RES (468)..(468): Leu or Arg
  MISC_FEATURE (474)..(493): Intracellular
  MISC_FEATURE (494)..(516): Transmembrane
  MOD_RES (502)..(502): Any amino acid
  MISC_FEATURE (517)..(536): Extracellular
  MISC_FEATURE (537)..(559): Transmembrane
  MOD_RES (540)..(540): Leu or Phe
  MOD_RES (553)..(553): Ile or Leu
  MOD_RES (554)..(554): Cys or Arg
  MOD_RES (556)..(556): Any amino acid
  MISC_FEATURE (560)..(581): Intracellular
  MOD_RES (565)..(565): Any amino acid
  MOD_RES (575)..(575): Asp or Gly
  MOD_RES (579)..(579): Ala or Val
  MISC_FEATURE (582)..(604): Transmembrane
  MOD_RES (582)..(582): Met or Ile
  MOD_RES (583)..(583): Ala or Val
  MOD_RES (586)..(586): Ile or Leu
  MOD_RES (588)..(588): Thr or Ile
  MOD_RES (589)..(589): Asp, His, Tyr, Gly, or Glu
  MOD_RES (592)..(592): Cys or Arg
  MOD_RES (600)..(600): Any amino acid
  MOD_RES (604)..(604): Ala or Phe
  MISC_FEATURE (605)..(615): Extracellular
  MISC_FEATURE (616)..(638): Transmembrane
  MOD_RES (623)..(623): Any amino acid
  MOD_RES (627)..(627): Ser or Tyr
  MOD_RES (636)..(636): Ile or Lys
  MISC_FEATURE (639)..(710): Intracellular
MKQRFSALQLLKLLLLLQLLKLLLLLQLQPPLPRALREALCPEPCNCVPDGALRCPGPTAGLTRLSLAYLPVKVIPSQAFRGLNEVIKIEISQIDSLERIEANAFDNLLNLSEILIQNTKNLRYXEPGAFINLPXLKYLSIXNTGIRKFPDVTKXFSSESNFILEICDNLHITTIPGNAFQGMNNESVTLKLYGNGFEEVQSHAXNGTTLTSLELKENVHLEKMHNGAFRGATGPKTLDISSTKLQALPSYGLESIQRLIATSSYSLKKLPSRETFVNLLEATLTYPSHCCAFRNLPTKEQXFSHSISENFSKQCESTVRKVXNKTLYSSMLAESELSGWDYEYGFCLPKTPRXAPEPDAFNPCXDIMGYDFLRVLIWXINILXXMGNMTVLFVLLTSRYKLXVPRFLXCNLSFADFCMGLYLLLIASVDSQTKGQYYNHAIDWQTGSGCSTAGFFTVFASELSVYTXTVITLERWHTITYAIHLDQKLRLRHAILIMLGGXLFSSLIAMLPLVGVSNYMKVSICFPMDVETTLSQVYIXTILILNVVAFFIXXAXYIKIYFAVXNPELMATNKXTKIXKKXXILXFXXFTXMAPISFFXISAXFKVPLITVTNSKVLLVLFXPINXCANPFLYAXFTKTFQRDFFLLLSKFGCCKRRAELYRRKDFSAYTSNCKNGFTGSNKPSQSTLKLSTLHCQGTALLDKTRYTEC

SEQ ID NO: 131 - protein, 113 aa, Artificial Sequence
  Description of Artificial Sequence Synthetic polypeptide
EVQLQESGPDLVKPSQSLSLTCTVTGYSITSGYGWHWIRQFPGNKLEWMGYIHYSGSTTYNPSLKSRISISRDTSKNQFFLQLNSVTTEDTATYYCARSLRYWGQGTTLTVSS

SEQ ID NO: 132 - protein, 115 aa, Artificial Sequence
  Description of Artificial Sequence Synthetic polypeptide
QVQLKESGPGLVAPSQSLSITCTVSGFSLTTYGVSWVRQPPGKGLEWLGVIWGDGSTYYHSALISRLSISKDNSKSQVFLKLNSLQTDDTATYYCAEGSSLFAYWGQGTLVTVSA

SEQ ID NO: 133 - protein, 121 aa, Artificial Sequence
  Description of Artificial Sequence Synthetic polypeptide
QVKLQQSGPELVKPGASVKISCKASGYSFTGYYMHWVKQSHGNILDWIGYIYPYNGVSSYNQKFKGKATLTVDKSSSTAYMELRSLTSEDSAVYYCARERGLYQLRAMDYWGQGTSVTVSS

SEQ ID NO: 134 - protein, 103 aa, Artificial Sequence
  Description of Artificial Sequence Synthetic polypeptide
EVQLEQSGGGLVQPGGSRKLSCAASGFTFSSFGMHWVRQAPEKGLEWVAYISSGSSTLHYADTVKGRFTISRDNPKNTLFLQMKLPSLCYGLLGSRNLSHRLL

SEQ ID NO: 135 - protein, 106 aa, Artificial Sequence
  Description of Artificial Sequence Synthetic polypeptide
DIVMTQTPAIMSASPGQKVTITCSASSSVNYMHWYQQKLGSSPKLWIYDTSKLAPGVPARFSGSGSGTSYSLTISSMEAEDAASYFCHQWSSYPYTFGSGTKLEIK

SEQ ID NO: 136 - protein, 107 aa, Artificial Sequence
  Description of Artificial Sequence Synthetic polypeptide
DIVLTQTPSSLSASLGDTITITCHASQNINVWLFWYQQKPGNIPKLLIYKASNLLTGVPSRFSGSGSGTGFTLTISSLQPEDIATYYCQQGQSFPWTFGGGTKLEIK

SEQ ID NO: 137 - protein, 113 aa, Artificial Sequence
  Description of Artificial Sequence Synthetic polypeptide
  MOD_RES (89)..(89): Any amino acid
DIVMTQSPSSLTVTAGEKVTMSCKSSQSLLNSGNQKNYLTWYQQKPGQPPKLLIYWASTRQSGVPDRFTGSGSGTDFTLTISSVQAEDXAVYYCQNDYSYPLTFGSGTKLEIK

SEQ ID NO: 138 - protein, 107 aa, Artificial Sequence
  Description of Artificial Sequence Synthetic polypeptide
DIVLTQTPATLSVTPGDSVSLSCRASQSISNNLHWYQQKSHESPRLLIKNASQSISGIPSKFSGSGSGTDFTLRINSVETEDFGMYFCQQSNSWPYTFGSGTKLEIK